SEQUENCE LISTING (13 sequences)

SEQ ID NO: 1 - protein, 45 aa, Artificial Sequence
  Description of Artificial Sequence Synthetic peptide
GGSGSGGQSGGSGSGGQSGGSGSGGQSGGSGSGGQSGGSGSGGQS

SEQ ID NO: 2 - DNA, 41 bp, Artificial Sequence
  Description of Artificial Sequence Synthetic oligonucleotide
aattcggtccggcgggagcggatccggcggccagtccccgc

SEQ ID NO: 3 - protein, 5 aa, Artificial Sequence
  Description of Artificial Sequence Synthetic peptide
SGLRS

SEQ ID NO: 4 - DNA, 26 bp, Artificial Sequence
  Description of Artificial Sequence Synthetic primer
acactatagaatgctagctacttgtt

SEQ ID NO: 5 - DNA, 24 bp, Artificial Sequence
  Description of Artificial Sequence Synthetic primer
agaggccttgaattcggacttgta

SEQ ID NO: 6 - DNA, 22 bp, Artificial Sequence
  Description of Artificial Sequence Synthetic primer
gacgagctgtacgaattcggcg

SEQ ID NO: 7 - DNA, 24 bp, Artificial Sequence
  Description of Artificial Sequence Synthetic primer
ctggaacaacactcaaccctatct

SEQ ID NO: 8 - DNA, 46 bp, Artificial Sequence
  Description of Artificial Sequence Synthetic oligonucleotide
cgcgccgagggccgccactccaccggcgccaaagaattcacgcgtg

SEQ ID NO: 9 - DNA, 46 bp, Artificial Sequence
  Description of Artificial Sequence Synthetic oligonucleotide
cgcgcacgcgtgaattctttggcgccggtggagtggcggccctcgg

SEQ ID NO: 10 - DNA, 3660 bp, Artificial Sequence
  CDS (1)..(3657)
  Description of Artificial Sequence Synthetic nucleotide construct
atggactgtctctgtatagtgacaaccaagaaataccgctaccaagatgaagacacgccccctctggaacacagcccggcccacctccccaaccaggccaattctccccctgtgattgtcaacacggacaccctagaagccccaggatatgagttgcaggtgaatggaacagagggggagatggagtatgaggagatcacattggaaaggggtaactcaggtctgggcttcagcatcgcaggtggcactgacaacccgcacatcggtgacgacccgtccatttttatcaccaagatcattcctggtggggctgcagcccaggatggccgcctcagggtcaatgacagcatcctgtttgtaaatgaagtggatgttcgggaggtgacccattcagctgcggtggaggccctcaaagaggcaggttccatcgttcgcctctatgtcatgcgccggaaacccccagccgaaaaggtcatggagatcaaactcatcaaagggcctaaaggacttggcttcagcattgcggggggcgttgggaaccagcacatccctggagataacagcatctatgtaacgaagatcatcgaaggaggtgctgcccacaaggatggcaggttgcagattggagacaagatcctggcggtcaacagtgtggggctggaggacgtcatgcacgaggatgccgtggcagccctgaagaacacatatgacgttgtgtacctaaaggtggccaagcccagcaatgcctacctgagtgacagctatgctcccccagacatcacaacctcgtattctcagcacctggacaatgagatcagtcatagcagctacttgggcactgactaccccacagccatgacccccacttcccctcggcgctactcccctgtggccaaggacctgctgggggaggaagacattccccgggaaccaaggcggatcgtgatccatcggggctccaccggcctgggcttcaacatcgtgggcggcgaggatggtgaaggcatcttcatctccttcatccttgctgggggtccagccgacctcagtggggagctacggaagggggaccagatcctgtcggtcaatggtgttgacctccgcaatgccagtcacgaacaggctgccattgccctgaagaatgcgggtcagacggtcacgatcatcgctcagtataaaccagaagagtatagtcgattcgaggccaagatccatgatcttcgggaacagctcatgaatagtagcctaggctcagggactgcatccttgcgaagcaaccccaagaggggcttctacattagggccctgtttgattacgacaagaccaaggactgcggtttcttgagccaggccctgagcttccgcttcggggatgtgcttcatgtcattgacgctggtgacgaagagtggtggcaagcacggcgggtccactccgacagtgagaccgacgacattggcttcattcccagcaaacggcgggtcgagcgacgagagtggtcaaggttaaaggccaaggactggggctccagctctggatcacagggtcgagaagactcggttctgagctatgagacggtgacccagatggaagtgcactatgctcgtcccatcatcatccttggacccaccaaagaccgtgccaacgatgatcttctctccgagttccccgacaagtttggatcctgtgtccctcatacgacacgtcctaagcgggaatatgagatagacggccgggattaccactttgtctcctcccgggagaaaatggagaaggacatccaggcacacaagttcattgaggctggccagtacaacagccacctctatgggaccagcgtccagtctgtgcgagaggtagcagagcaggggaagcactgcatcctcgatgtctcggccaatgccgtgcggcggctgcaggcggcccacctgcaccccatcgccatcttcatccgtccccgctccctggagaatgtgctagagatcaataagcggatcacagaggagcaagcccggaaagccttcgacagagccacgaagctggagcaggagttcacagagtgcttctcagccatcgtagagggcgacagctttgaagagatctatcacaaagtgaaacgtgtcattgaagacctctcaggcccctacatctgggtcccagcccgagagagactctccaattcggtccggcgggagcggatccggcggccagtccccgcgggccccaccatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtccggcgggagcggatccggcggccagtccggcgggagcggatccggcggccagtccggcgggagcggatccggcggccagtccggcgggagcggatccggcggccagtccggcgggagcggatccggcggccagtccggactcagatctgtcaaacttacatcagacttcgacaacccaagatggattggacgacacaagcatatgttcaatttccttgatgtcaaccacaatggaaaaatctctcttgacgagatggtctacaaggcatctgatattgtcatcaataaccttggagcaacacctgagcaagccaaacgacacaaagatgctgtggaagccttcttcggaggagctggaatgaaatatggtgtggaaactgattggcctgcatatattgaaggatggaaaaaattggctactgatgaattggagaaatacgccaaaaacgaaccaaccctcatccgcatctggggtgatgctttgtttgatatcgttgacaaagatcaaaatggagctattacactggatgaatggaaagcatacaccaaagctgctggtatcatccaatcatcagaagattgcgaggaaacattcagagtgtgcgatattgatgaaagtggacaactcgatgttgatgagatgacaagacagcatctgggattttggtacaccatggatcctgcttgcgaaaagctctacggtggagctgtcccctaa

SEQ ID NO: 11 - protein, 1219 aa, Artificial Sequence
  Description of Artificial Sequence Synthetic protein
MDCLCIVTTKKYRYQDEDTPPLEHSPAHLPNQANSPPVIVNTDTLEAPGYELQVNGTEGEMEYEEITLERGNSGLGFSIAGGTDNPHIGDDPSIFITKIIPGGAAAQDGRLRVNDSILFVNEVDVREVTHSAAVEALKEAGSIVRLYVMRRKPPAEKVMEIKLIKGPKGLGFSIAGGVGNQHIPGDNSIYVTKIIEGGAAHKDGRLQIGDKILAVNSVGLEDVMHEDAVAALKNTYDVVYLKVAKPSNAYLSDSYAPPDITTSYSQHLDNEISHSSYLGTDYPTAMTPTSPRRYSPVAKDLLGEEDIPREPRRIVIHRGSTGLGFNIVGGEDGEGIFISFILAGGPADLSGELRKGDQILSVNGVDLRNASHEQAAIALKNAGQTVTIIAQYKPEEYSRFEAKIHDLREQLMNSSLGSGTASLRSNPKRGFYIRALFDYDKTKDCGFLSQALSFRFGDVLHVIDAGDEEWWQARRVHSDSETDDIGFIPSKRRVERREWSRLKAKDWGSSSGSQGREDSVLSYETVTQMEVHYARPIIILGPTKDRANDDLLSEFPDKFGSCVPHTTRPKREYEIDGRDYHFVSSREKMEKDIQAHKFIEAGQYNSHLYGTSVQSVREVAEQGKHCILDVSANAVRRLQAAHLHPIAIFIRPRSLENVLEINKRITEEQARKAFDRATKLEQEFTECFSAIVEGDSFEEIYHKVKRVIEDLSGPYIWVPARERLSNSVRRERIRRPVPAGPTMSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITHGMDELYKSGGSGSGGQSGGSGSGGQSGGSGSGGQSGGSGSGGQSGGSGSGGQSGLRSVKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGQLDVDEMTRQHLGFWYTMDPACEKLYGGAVP

SEQ ID NO: 12 - DNA, 1539 bp, Artificial Sequence
  CDS (1)..(1536)
  Description of Artificial Sequence Synthetic nucleotide construct
atgtccgtcctgacgccgctgctgctgcggggcttgacaggctcggcccggcggctcccagtgccgcgcgccaagatccattcgttgctgcagccgcgggccaccatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtccggcgggagcggatccggcggccagtccggcgggagcggatccggcggccagtccggcgggagcggatccggcggccagtccggcgggagcggatccggcggccagtccggcgggagcggatccggcggccagtccggactcagatctgtcaaacttacatcagacttcgacaacccaagatggattggacgacacaagcatatgttcaatttccttgatgtcaaccacaatggaaaaatctctcttgacgagatggtctacaaggcatctgatattgtcatcaataaccttggagcaacacctgagcaagccaaacgacacaaagatgctgtggaagccttcttcggaggagctggaatgaaatatggtgtggaaactgattggcctgcatatattgaaggatggaaaaaattggctactgatgaattggagaaatacgccaaaaacgaaccaaccctcatccgcatctggggtgatgctttgtttgatatcgttgacaaagatcaaaatggagctattacactggatgaatggaaagcatacaccaaagctgctggtatcatccaatcatcagaagattgcgaggaaacattcagagtgtgcgatattgatgaaagtggacaactcgatgttgatgagatgacaagacagcatctgggattttggtacaccatggatcctgcttgcgaaaagctctacggtggagctgtcccctaa

SEQ ID NO: 13 - protein, 512 aa, Artificial Sequence
  Description of Artificial Sequence Synthetic protein
MSVLTPLLLRGLTGSARRLPVPRAKIHSLLQPRATMSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITHGMDELYKSGGSGSGGQSGGSGSGGQSGGSGSGGQSGGSGSGGQSGGSGSGGQSGLRSVKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGQLDVDEMTRQHLGFWYTMDPACEKLYGGAVP

(19) United States
(12) Patent Application Publication
(10) Pub. No.: US 2008/0282362 A1
(43) Pub. Date: Nov. 13, 2008

(54) Non-invasive real-time in vivo bioluminescence imaging of local Ca²+ dynamics in living organisms

(76) Inventors: Philippe Brulet, Paris (FR); Kelly Rogers, Paris (FR); Thomas Curie, Bouchenaine (FR)

Correspondence Address: FINNEGAN, HENDERSON, FARABOW,;GARRETT & DUNNER, L.L.P., 901 New York Avenue, NW, Washington, DC, 20001-4413, US

(21) Appl. No.: 11/509,060

(22) Filed: Aug. 24, 2006

Related U.S. Application Data
(63) Continuation-in-part of application No. 11/032,236, filed on Jan. 11, 2005.
(60) Provisional application No. 60/543,659, filed on Feb. 12, 2004.

(51) Int. Cl. A01K 67/027; A01H 1/00

(52) U.S. Cl. 800/3; 800/18; 800/282

(57) ABSTRACT

A method for bioluminescence imaging in an animal is provided. The method comprises providing a whole animal containing a transcriptionally active nucleic acid sequence encoding a Ca²⁺-sensitive polypeptide, which comprises a chemiluminescent protein linked to a fluorescent protein; and monitoring photons emitted by the Ca²⁺-sensitive polypeptide. The Ca²⁺-sensitive polypeptide comprises aequorin protein covalently linked to a YFP (yellow fluorescent protein) or RFP (red fluorescent protein), and the link between the two proteins functions to allow luminescence by energy transfer between the two proteins. The photons are monitored from deep tissues of the animal.

### CROSS-REFERENCE TO RELATED APPLICATIONS

This application is a continuation-in-part of and claims the benefit of U.S. patent application Ser. No. 11/032,236, filed Jan. 11, 2005 (Attorney Docket No. 03495.0328), and is also based on and claims the benefit of U.S. Provisional Application No. 60/543,659, filed Feb. 12, 2004, (Attorney Docket No. 3495.6097). The entire disclosure of each of these applications is relied upon and incorporated by reference herein.

### BACKGROUND OF THE INVENTION

This invention provides a method to permit optical detection of localised calcium signaling (e.g. high Ca²⁺ concentration microdomains) using a genetically encoded bioluminescent reporter. This invention describes a method to detect the effect of a pharmacological agent or neuromodulator on localised Ca²⁺ signalling. The invention especially provides a method to visualise dynamic fluctuations in localised Ca²⁺ associated with cell or tissue activation, such as neuronal activation and relating to optical detection of ion channel function (receptors/channels permeable to Ca²⁺) and synaptic transmission. This invention also concerns a method for optical detection of the dynamics of Ca²⁺ in a biological system, said method comprising monitoring the photons emitted by a recombinant Ca²⁺-sensitive polypeptide, which comprises or consists of a chemiluminescent protein linked to a fluorescent protein, present in said biological system. Also, this invention provides a transgenic non-human animal expressing a recombinant polypeptide sensitive to calcium concentration, consisting of at least a chemiluminescent protein linked to a fluorescent protein, in conditions enabling the in vivo monitoring of local calcium dynamics.

Ca²⁺ is one of the most universal and physiologically important signaling molecules that plays a role in almost all cellular functions, including fertilization, secretion, contraction-relaxation, cell motility, cytoplasmic and mitochondrial metabolism, synthesis, production of proteins, gene expression, cell cycle progression and apoptosis (Rizzuto et al., 2002).

Characteristics of Ca²⁺ transients at the cellular and subcellular level are complex, and vary according to spatial, temporal and quantitative factors. Up to a 20,000-fold difference in the concentration of Ca²⁺ exists between the cytoplasm and the extracellular space, such that even when channels are open for a short time, a high rate of Ca²⁺ influx will occur. Factors such as diffusion, Ca²⁺ binding to buffer proteins and sequestration by cellular compartments, will create a Ca²⁺ gradient and result in a high concentration microdomain within a few hundred nanometers from the pore of a channel. Over longer distances such as tens of microns, the effective diffusion coefficient of Ca²⁺ will be strongly reduced.

Because Ca²⁺ signals are highly regulated in space, time and amplitude, they have a defined profile (e.g. amplitude and kinetics). Ca²⁺ transients are shaped by cytosolic diffusion of Ca²⁺, buffering by Ca²⁺ binding proteins and Ca²⁺ transport by organellar (Bauer, 2001; Llinas et al., 1995). The concentration of Ca²⁺ reached and its kinetics in any given cellular microdomain is critical for determining whether a signaling pathway succeeds or not in reaching its targets. Ca²⁺ is necessary for activation of many key cellular proteins, including enzymes such as kinases and phosphatases, transcription factors and the protein machinery involved in secretion. Ca²⁺ signaling cascades may also mediate negative feedback on the regulation of biochemical pathways or functional receptors and transport mechanisms. The propagation of Ca²⁺ within a cell can also help to link local signaling pathways to ones that are more remote within a cell or for facilitating long distance communication between cells or networks of cells (e.g. central nervous system) (Augustine et al., 2003).

Ca²⁺ transients producing high Ca²⁺ concentration microdomains are associated with a diverse array of functions important in development, secretion and apoptosis, and many cellular processes, including gene expression, neurotransmission, synaptic plasticity and neuronal cell death (Augustine et al., 2003; Bauer, 2001; Llinas et al., 1995; Neher, 1998). Characterising the spatiotemporal specificity of Ca²⁺ profiles is important to understand the mechanisms contributing to perturbed cellular Ca²⁺ homeostasis, which has been implicated in many pathological processes, including migraine, schizophrenia and early events associated with the onset of neurodegenerative diseases such as Alzheimer's, Parkinson's and Huntington's diseases (Mattson and Chan, 2003). Because Ca²⁺ is directly or indirectly associated with almost all cell signaling pathways, optical detection of Ca²⁺ is a universal measure of biological activity at the molecular, cellular, tissue and whole animal level.

Tremendous progress has been made in the imaging of localised Ca²⁺ events using light microscopy. To this end, Ca²⁺ signalling in single dendritic spines (Yuste, 2003) and more recently in a single synapse (Digregorio, 2003) has been accomplished using fluorescent dyes. However, one way to spatially improve measurements of Ca²⁺ is to genetically target a reporter protein to a specific location whereby Ca²⁺ activity can be directly visualised. Specifically, such a reporter protein could be fixed in a microdomain (within 200 nm of the source or acceptor) or even within a nanodomain (within 20 nm) (see Augustine et al. 2003 for review). Expression of a reporter gene under the control of cell type-specific promoters in transgenic animals, can also offer a non-invasive way to follow dynamic changes in a single cell type, tissues or anatomically in whole animal imaging.

Monitoring calcium in real-time can help to improve the understanding of the development, the plasticity and the functioning of a biological system, for example the central nervous system. Indeed, much effort has been dedicated to the development of an optical technique to image electrical activity in single-cell type and particularly single neurons and networks of neurons, but there continues to be a need to achieve this goal through use also of electrophysiological techniques. Genetic targeting of a Ca²⁺ reporter probe in spatially restricted areas of a cell or living system (e.g. inside of a compartment, to microdomains or nanodomains, or by fusion to a specific polypeptide) is a molecular imaging approach for detecting specific cellular activities or physiological functions.

### SUMMARY OF THE INVENTION

This invention aids in fulfilling these needs in the art, by providing a method for optical detection of the dynamics of Ca²⁺ in a biological system, said method comprising monitoring the photons emitted by a recombinant Ca²⁺-sensitive polypeptide, which comprises or consists of a chemiluminescent protein linked to a fluorescent protein, present in said biological system, as well as a transgenic non-human animal expressing said recombinant polypeptide sensitive to calcium. The non-invasive nature of this technique as well as the evidence that the recombinant protein is non-toxic, means that the method could possibly also be applied in humans.

More particularly, this invention provides a method for bioluminescence imaging in a biological system. The method comprises providing a biological system containing a transcriptionally active nucleic acid sequence encoding a Ca²⁺-sensitive polypeptide, or a Ca²⁺-sensitive polypeptide, which comprises a chemiluminescent protein linked to a fluorescent protein; and monitoring photons emitted by the Ca²⁺-sensitive polypeptide. The Ca²⁺-sensitive polypeptide comprises a chemiluminescent protein sensitive to Ca²⁺ linked to a yellow fluorescent protein or a red fluorescent protein. The link between the two proteins functions to allow luminescence by energy transfer between the two proteins. In alternative embodiments, the chemiluminescent protein, which is sensitive to Ca²⁺, is covalently linked to the yellow fluorescent protein or red fluorescent protein, and the chemiluminescent protein, which is sensitive to Ca²⁺, can be aequorin. In preferred embodiments, the yellow fluorescent protein is the Venus yellow fluorescent protein, and the red fluorescent protein is mRFP1. The photons emitted by the Ca²⁺-sensitive polypeptide are monitored in an animal or a plant.

In a preferred embodiment, the photons emitted by the Ca²⁺-sensitive polypeptide are monitored from deep tissues of an animal. In the present application, “deep tissue” means tissue under muscles or under the skull. Examples of such tissues are the brain, the liver, the lung, the heart, and tissues of the vascular system. Deep tissues include, more generally, a subthoracic tissue or a subcranial tissue.

In another preferred embodiment, the animal or plant is a transgenic animal, such as a transgenic mouse, or a transgenic plant.

This invention also provides a method for the optical detection of Ca²⁺ signals in a biological system, wherein the method comprises providing a biological system containing a transcriptionally active nucleic acid sequence encoding a Ca²⁺-sensitive polypeptide, or a Ca²⁺-sensitive polypeptide, which comprises a chemiluminescent protein linked to a fluorescent protein; and monitoring photons emitted by the Ca²⁺-sensitive polypeptide. The Ca²⁺-sensitive polypeptide comprises a chemiluminescent protein, which is sensitive to Ca²⁺, linked to a yellow fluorescent protein or red fluorescent protein. The link between the two proteins functions to allow luminescence by energy transfer between the two proteins.

In addition, this invention provides a method for the optical detection of Ca²⁺ signals in an animal, wherein the method comprises providing a whole, live, animal containing a transcriptionally active nucleic acid sequence encoding a Ca²⁺-sensitive polypeptide, or a Ca²⁺-sensitive polypeptide, which comprises a chemiluminescent protein linked to a fluorescent protein; and non-invasively monitoring photons emitted by the Ca²⁺-sensitive polypeptide. The Ca²⁺-sensitive polypeptide comprises aequorin protein linked to a yellow fluorescent protein or a red fluorescent protein, and the link between the two proteins functions to allow transfer of energy by radiative or non-radiative intramolecular energy transfer. In a preferred embodiment, the chemiluminescent protein, which is sensitive to Ca²⁺, is covalently linked to the yellow fluorescent protein or red fluorescent protein. The link between the two proteins can function to allow transfer of energy by Chemiluminescence Resonance Energy Transfer (CRET) between the two proteins. An example of a yellow fluorescent protein is the Venus yellow fluorescent protein, and an example of a red fluorescent protein is mRFP1.

A further embodiment of the invention provides a method for the optical detection of Ca²⁺ signals in a transgenic mouse. The method comprises providing a freely moving, whole, live, transgenic mouse containing a transcriptionally active transgene encoding a Ca²⁺-sensitive polypeptide, which comprises a chemiluminescent protein linked to a fluorescent protein; and non-invasively monitoring photons emitted by the Ca²⁺-sensitive polypeptide. The Ca²⁺-sensitive polypeptide comprises aequorin protein covalently linked to a YFP (yellow fluorescent protein) or RFP (red fluorescent protein), and the link between the two proteins functions to allow transfer of energy by Chemiluminescence Resonance Energy Transfer (CRET) between the two proteins. The photons are monitored from subthoracic tissue or subcranial tissue of the transgenic mouse. Optionally, the photons can be monitored during motion of the transgenic mouse.

In one embodiment of the invention the aequorin is covalently linked to YFP and the photons are monitored from subthoracic tissue. The subthoracic tissue comprises the heart in a preferred embodiment.

In another embodiment the aequorin is covalently linked to RFP and the photons are monitored from subcranial tissue or liver. The transgenic mouse can be an adult transgenic mouse, and the photons can be monitored through the skull of the transgenic mouse. A preferred embodiment of the invention comprises monitoring photons having a wavelength greater than about 600 nm emitted by the Ca²⁺-sensitive polypeptides.

Thus, this invention relates to a method for bioluminescence imaging or optical detection of Ca²⁺ in a biological system using a transcriptionally active nucleic acid sequence coding for a sensitive polypeptide, or a Ca²⁺-sensitive polypeptide, the chemiluminescent protein being sensitive to Ca²⁺, the fluorescent protein being a yellow fluorescent protein or a red fluorescent protein. The methods for bioluminescence imaging or optical detection of Ca²⁺ in an animal or a transgenic animal, the use of aequorin and YFP or RFP, are the methods for monitoring photons from subthoracic tissue or subcranial tissue are preferred embodiments of the invention.

### BRIEF DESCRIPTION OF THE DRAWINGS

This invention will be described with reference to the following drawings:

FIG. 1: Schematic diagram showing the localisation of the different GFP-Aequorin reporters targeted to specific subcellular domains in the pre- and post-synaptic compartment. The GFP-aequorin reporter has been targeted to different cellular domains, including the mitochondrial matrix (mtGA), by fusion to synaptotagmin I (SynGA), to the lumen of the endoplasmic reticulum (erGA) and by fusion to PSD95 (PSDGA). The low-affinity version of each reporter could allow selective detection of high-calcium concentration microdomains that are indicative of specific cellular activities.

FIG. 2: Schematic representation of the different GA chimeras for cell specific targeting. The white asterisk shows the position of the (Asp-119 Ala) mutation in aequorin, reducing the Ca²⁺ binding affinity of the photoprotein, as described by Kendall et al, 1992. GA represents non-targeted GFP-Aequorin, denoted G5A, and containing a flexible linker between the two proteins (GA and SynGA are declared in the application PCT/EP01/07057). mtGA, mitochondrially targeted GFP-aequorin by fusing GA to the cleavable targeting sequence of subunit VIII of cytochrome c oxidase; erGA, GFP-aequorin targeted to the lumen of the endoplasmic reticulum after fusion to the N-terminal region of the immunoglobulin heavy chain, PSDGA, fusion of GFP-aequorin to PSD95 for localised targeting in postsynaptic structures. All constructs are under the control of the human cytomegalovirus promoter (pCMV).

FIG. 3: Ca²⁺ concentration response curves for mtGA. Ca²⁺ concentration response curves for mtGA after reconstitution of the recombinant protein with the native or the synthetic analog, h coelenterazine, which is reported to be more sensitive to Ca²⁺ than is the native complex. (determined at pH 7.2 and 26° C. (n=3)). The fractional rate of aequorin consumption is proportional in the physiological pCa range, to [Ca²⁺]. The fractional rate of photoprotein consumption is expressed as the ratio between the emission of light at a defined [Ca²⁺] (L) and the maximal light emission at a saturating [Ca²⁺] (Lmax).

FIG. 4: Confocal microscopy analysis of the different GFP-Aequorin chimeras targeted to specific subcellular domains. (A) mtGA, GFP-Aequorin is well targeted to the mitochondrial matrix in COS7 and cortical neurons. (B) erGA, GFP-Aequorin is well targeted to the lumen of the endoplasmic reticulum (C) PSDGA, GFP-Aequorin fused to the C-terminus of the PSD95 protein, results in punctuate labeling of the Ca²⁺-reporter that resembles targeting of the native protein in dissociated cortical neurons. (D) SynGA, GFP-Aequorin fused to the C-terminus of synaptotagmin I, the synaptic vesicle transmembrane protein, labels synaptic regions. Targeted GA reporters have also been verified by immunohistochemical staining with relevant antibodies.

FIG. 5. Cortical cells transfected with the non-targeted version of GFP-Aequorin. GFP-aequorin were reconstituted with the high affinity h version of coelenterazine. (A) GFP fluorescence shows homogenous distribution of the Ca²⁺ reporter. Ca²⁺ induced bioluminescence and corresponding graphical data after application of (B, b.) 100 μM NMDA and (C, c.) 90 mM KCl to a single cortical neuron transfected with GA. (D) High Ca²⁺ solution containing digitonin was added at the end of the experiment to quantitate the total amount of photoprotein for calibration of the Ca²⁺ concentration. Images were obtained at room temperature (23-25° C.) using a ×40 objective with a 1.3 NA. Scale bar=15 μm Changes in [Ca²⁺] as indicated by the number of photons detected, are coded in pseudocolor (1-5 photons/pixel), where dark blue represents low and red represents high pixel counts.

FIG. 6: NMDA induced influx of Ca²⁺ in a cortical neuron transfected with mtGA. GFP enables the expression patterns of the Ca²⁺ reporter to be visualized by fluorescence microscopy as shown in the first image, baseline, where the GFP fluorescence image has been superimposed with the photon image prior to stimulation. Using a highly sensitive image photon detector (IPD), Ca²⁺ induced bioluminescence was recorded after application of NMDA (100 μM. IPD detection provides a high degree of temporal resolution and a moderate degree of spatial resolution. See the zoomed region showing a comparison of the spatial resolution between (A) the CCD fluorescence image, scale bar=5 μm and (B) the IPD photon image. (C) GFP fluorescence image showing regions of interest and corresponding graphical data. Scale bar=10 μm). A graph is represented also for the whole cell response. Each photon image represents 30 seconds of accumulated light. Background <1 photon/sec. The color scale represents luminescence flux as 1-5 photons/pixel.

FIG. 7: Ca²⁺ induced bioluminescence activity in a cortical neuron transfected with SynGA. In basal conditions, before the addition of a neuromodulator, regions analysed showed a higher level of activity in comparison to background. This is consistently observed in neurons transfected with SynGA. Normally, it is difficult to detect resting levels of Ca²⁺ when GFP-Aequorin is regenerated with native aequorin, given the low binding affinity of the reporter. mtGA and PSDGA, do not generally exhibit the same kind of activity, although PSDGA sometimes shows very localized Ca²⁺ fluxes that occur spontaneously and in a stochastic fashion. These results suggest that SynGA is targeted to a cellular domain that is higher in Ca²⁺ than normally reported for resting levels of cytosolic Ca²⁺ Background photons were less than 1 photon/sec in the 256×256 pixel region. 20×20 pixel regions were selected from the cell soma and various places along the neurites. Graphical data also shows the increase in background counts for each region. Note, that background is very close to zero, so it is not seen. Influx of Ca²⁺ in the cell soma and neurites after addition of high K+ (90 mM KCl). Corresponding (BF) brightfield and (Fl) fluorescence images are shown as well as the superimposition of the photon image with the fluorescence image. Scale bar=20 μm. Photon images were scaled for 1-5 photons/pixel.

FIG. 8: Cortical neurons transfected with PSDGA. (A) GFP fluorescence was visualized to identify those neurons showing expression of the Ca²⁺ reporter, which resembles that of the native PSD95 protein. Photon emission in two dendritic regions (15×15 pixels), denoted D1 and D2 and in the same size region from the cell soma, were investigated and are graphically represented. The dynamics of Ca²⁺ signaling was found to be identical in the two dendritic regions analysed, but markedly different in comparison to the cell soma. (B) Photon image showing the total integration (50-200 s) of photons emitted after the first application of NMDA. Photons were only detected in the cell soma region after a second application of NMDA as the total photoprotein in the two dendritic regions analysed was completely consumed after the first application of NMDA. The pseudo-color scale represents 1-5 photons/pixel. Scale bar=10 μm.

FIG. 9: Observation of spontaneous activity recorded from a cortical cell expressing PSDGA (GFP-Aequorin fused to PSD95). Responses were recorded under basal conditions and are graphically represented (colors represent data collected from the same 20×20 pixel region, each pixel=0.65 μm). Corresponding examples are demonstrated and include the integrated photon image and graphical data.

FIG. 10: Long-term bioluminescence imaging of Ca²⁺ dynamics in an organotypic hippocampal slice culture from neonatal mouse brain, infected with an adenoviral-GFP-Aequorin vector. (A) GFP fluorescence shows individual cells expressing the Ca²⁺ reporter. Activity was recorded for a period of approximately 9 hours before cell death became apparent as indicated by a large increase in bioluminescence activity and loss of fluorescence. Fluorescence images were taken periodically (each 30 min) throughout the acquisition. Representative photon images are shown as well as the corresponding graphical data (last 7 hours). Background <1 photon/sec.×10.

FIG. 11: (A) Map of the PSDGA vector and (B) the coding sequence (and corresponding protein sequence) of the insert comprising PSD95 (nucleotide positions 616 to 2788), an adaptor (capital letters), GFP (2842 to 3555), a linker (3556 to 3705, capital letters) and the aequorin (3706 to 4275).

FIG. 12: (A) Map of the mtGA vector and (B) coding sequence (and corresponding protein sequence) of the insert comprising the cleaveable targeting sequence of subunit VIII of cytochrome C oxidase (nucleotide positions 636 to 722, capital letters), GFP (741 to 1454), a linker (1455 to 1604, capital letters) and the aequorin (1605 to 2174).

FIG. 13: Detection of dynamic activity in single-cells when GFP-aequorin is localized to specific cellular domains. Ca²⁺-induced bioluminescence in a cortical neuron transfected with PSDGA. The propagation of Ca²⁺ waves and response profiles produced subcellularly were shown to be highly complex. The IPD camera used in these studies provides μs time resolution and integration times are specified only for on-line visualization. Working with a highly variable time scale enables the full extent of the spatiotemporal properties of Ca²⁺ activity to be investigated, which is itself a physiological parameter. Scale bar=20 μm.

FIG. 14: Electrically induced Ca²⁺-oscillations in hippocampal neurons. Fluorescence (Fl) and brightfield images (BF) from a 24 day old culture, whereby a patch-like pipette (7-10 MD) connected to a pulse generator was brought in gentle contact with the somatic region of the ‘lower’ cell shown in the image. Scale bar=20 μm. Light emission induced by a single 2 ms electrical pulse (A) is shown in 5 s frames and in A to E graphs shown on the lower panel. The applied voltage is given on each graph (polarity refers to the battery side the pipette is connected to). The photon images are superimposed with the brightfield image. The fractional light emission (L/Lmax) from the 3 regions indicated in BF appears in the graphs A to E and correspond to successive electrical stimulations. (F) All Ca²⁺ transients recorded in each region of interest during a 20-minute period are shown as a function of time. The asterisk indicates the first of a series of spontaneously occurring transients. (G) Schematic diagram of the electrical arrangement. I.S., isolated stimulator, R, electrical relay, A, patch amplifier head. Neurons were transfected with a viral vector containing the GFP-aequorin gene (see Methods for more detail).

FIG. 15: GFP fluorescence of 10.5 day old transgenic embryos vs wildtype embryos. Chimeric mtGA (pCAG-Lox-stop-Lox-mtGA) mice were crossed with a PGK-CRE mouse to activate expression of the transgene in all cells of the body from the beginning of development. (A) Brightfield images of a 10.5 day old embryo from wild-type and transgenic mice; (B) Corresponding GFP fluorescence images. No phenotypic abnormalities are apparent.

FIG. 16: GFP fluorescence in neonatal transgenic mice expressing the mitochondrially targeted GFP-acquorin protein in all cells. Brightfield and corresponding GFP fluorescence images of mtGA mouse after activation of the transgene (by crossing with PGK-CRE) in all cells of the body from the beginning of development. (A) Foot, (B) Dorsal view of the upper body, (C1) Dorsal view of the head and (C2) targeting of mtGA in cortex of P1 mtGA mouse. No physical or behavioral abnormalities are apparent in newborn or adult mice.

FIG. 17: GFP fluorescence images of organs excised from transgenic neonatal mice versus organs from wildtype mice. GFP fluorescence images of P5 transgenic mtGA vs wild-type mouse. Images were taken of the major organs and show strong levels of expression in all organs. Highest levels of expression are apparent in the heart and liver. No abnormalities in the organs are apparent when the transgene is activated at the beginning of development in all cells.

FIG. 18: Confocal analysis of mtGA in cortex of transgenic animals expressing the transgene in all cells. Transgenic mice were crossed with PGK-CRE mice for activated expression of the mtGA transgene in all cells of the living animal. Organotypic slices were prepared from P4 mouse and kept in culture for 4 days before undertaking experiments to detect Ca²⁺-induced bioluminescence. At the completion of the experiment, slices were fixed and then stained for GFAP (for detection of glial cells) and NeuN (for detection of neurons). Both antibodies colocalize to expression of the mtGA transgene. GFP fluorescence shows expected expression patterns of the GA reporters after fusion to the signal peptide of cytochrome c for targeting to the mitochondrial matrix (mtGA). These results show that the mtGA transgene is expressed in all cells of the brain when transgenic mtGA reporter mice are crossed with PGK-CRE mice, which activates Cre in all cells.

FIG. 19: Synchronized oscillations of mitochondrial Ca²⁺ transients in the somatosensory cortex of the immature mouse brain. (BF, brightfield & FI, fluorescence images) Organotypic slices (coronal) were cut from P4 transgenic mice expressing the mitochondrially targeted GFP-aequorin in all cells of the brain. Slices were kept in culture for 4-5 days before imaging. After incubation with coelenterazine (wt), slices were perfused in a buffer (with or without Mg2+). The results for two slices are represented, showing that removal of Mg2+ from the buffer generates Ca²⁺ oscillations that are detected from within the mitochondrial matrix and that are completely and reversibly blocked by the NMDA antagonist, D-APV (50 μM). Photons were collected from a 550 μm2 region corresponding to somato sensory cortex in layers I-III/IV and V of the cerebral cortex.

FIG. 20: Whole animal bioluminescence imaging of P1 mice with mitochondrially targeted GFP-aequorin. The mouse on the left handside is a wild-type mouse and the mouse on the right handside is a transgenic mouse expressing mitochondrially targeted GFP-aequorin in all cells. Both mice have been injected intraperitoneally with coelenterazine (4 μg/g). A-C, represent separate sequences where consecutive images were acquired over time. A grayscale photograph of the mice was first collected in the chamber under dim light emitting diode illumination, followed by the acquisition and overlay of the pseudocolor luminescent image. Each frame represents 5 seconds of light accumulation. Color bars corresponding to the light intensity from violet (least intense) to red (most intense) is given at the end of each sequence. Scale bar=2 cm.

FIG. 21: Whole animal bioluminescence imaging of a P3 mouse with mitochondrially targeted GFP-aequorin. The mouse on the left handside is a wild-type mouse and the mouse on the right handside is a transgenic mouse expressing mitochondrially targeted GFP-aequorin in all cells. Both mice have been injected intraperitoneally with coelenterazine (4 μg/g). A & B represent separate sequences where consecutive images were acquired over time. A grayscale photograph of the mice was first collected in the chamber under dim light emitting diode illumination, followed by the acquisition and overlay of the pseudocolor luminescent image. Each frame represents 5 seconds of light accumulation. Color bars corresponding to the light intensity from violet (least intense) to red (most intense) is given at the end of each sequence. Scale bar=4 cm.

FIG. 22: Whole animal bioluminescence imaging of mitochondrial Ca²⁺ dynamics with higher time resolution. The mouse on the left handside is a wild-type mouse and the mouse on the right handside is a transgenic mouse expressing mitochondrially targeted GFP-aequorin in all cells. Both mice have been injected intraperitoneally with coelenterazine (4 μg/g). Exposure times are indicated on each consecutive frame, ranging from 2-5 seconds.

FIG. 23: Whole animal bioluminescence imaging of mitochondrial Ca²⁺ with higher time resolution. The mouse on the left handside is a wild-type mouse and the mouse on the right handside is a transgenic mouse expressing mitochondrially targeted GFP-aequorin in all cells. Image represents 1 second of light accumulation. Color bar corresponds to the intensity of light. The image was acquired using the Xenogen IVIS100 whole animal bioluminescence system. Binning=16, F/stop=1.

FIG. 24: Schematic representation of the different hybrid genes corresponding to the different photoproteins. Each construct was under the control of the CMV promoter. Color coding in this figure also apply to the other figures.

FIG. 25: Fluorescence excitation and emission spectra of the different fluorescent hybrid proteins when expressed in Cos7 cells. The excitation spectrum is shown with a solid line and the emission spectrum is shown with a dotted line.

FIG. 26: Characteristics of the chimeric photoproteins: (A) Kinetic response of mRFP1-aequorin hybrid reconstituted photoprotein in presence of different free [Ca²⁺]. Values are given as a logarithmic of the total photons recorded. (B) [Ca²⁺] response curve for each hybrid protein reconstituted with the wt version of coelenterazine, determined at pH 7.2 and 25° C. (r²=0.99, n=3). (C) Stability of reconstituted cytosolic hybrid photoproteins over time, at room temperature. Each point represents the total light produced by the different proteins upon the addition of 100 mM CaCl₂ solution as a function of time. The results were fitted by a Boltzman's distribution using Graph Pad Prism4® software. Values are given as the percentage of total photons remaining compared to time zero and are the mean +/−SEM (n=4 for each photoprotein). (D) pH titration of the light intensity in the presence of 10 mM CaCl₂. The values are relative to the total amount of light emitted for each photoprotein and have been fitted to a 4^{th} order polynomial curve. (B-D), Green line=GFP-aequorin (GA); yellow line=Venus-aequorin (VA), red line=mRFP1-aequorin (RA) and blue line=aequorin (Aeq).

FIG. 27: [Ca²⁺] Chemiluminiscence Resonance Energy Transfer (CRET) activities on cellular extracts corresponding to GA (green line), VA (yellow line), RA (red line) and Aeq (blue line). CRET emission spectra of aequorin and the hybrid photoproteins were analysed from 350 to 750 nm over 20 seconds at an acquisition rate of 2 Hz and calibrated as a percentage of total light. Notice that RA also emits signals at wavelengths around 600 nm.

FIG. 28: Light intensity emitted from each photoprotein in the presence of 866 nM free Ca²⁺ as recorded through selected band pass or long pass filters using the Xenogen IVIS system. The grey scale video image is superimposed with the colour coded bioluminescent images showing the transmission of light from each photoprotein through different band-pass (BP) and long-pass-filters (LP).

FIG. 29: Superimposed visible light and colour coded bioluminescent images of mice in which a “small” tube containing 50 μl photoprotein in 866 nM Ca²⁺-buffered solution has been placed subcutaneously (top row) or subthoracically (bottom row). Bioluminescent signals have been acquired over 5 minutes. All photoproteins have been calibrated to the same signal intensity before whole animal imaging and also to a reference tube located laterally. Bioluminescence colour coding as in FIG. 28.

FIG. 30: Superimposed video and colour coded bioluminescent images of mice in which a “small” tube containing 50 μl photoprotein in 866 nM Ca²⁺-buffered solution has been placed subcranially. Bioluminescent signals have been acquired over 5 minutes. All photoproteins have been calibrated for the same signal intensity before whole animal imaging. RA+Filter has a bandpass=610-630 nm wavelength. Bioluminescence colour coding as in FIG. 28.

### DESCRIPTION OF THE INVENTION

Among the coelenterates, bioluminescent species exist. Numerous studies have shown that the bioluminescence is generated by photoproteins that are often sensitive to calcium. Sensitive as used herein when referring to a protein means any modification in said sensitive protein in its conformation, affinity for other molecules, localisation, or in the emission of light. Particular proteins of this type emit a flash of light in response to an increase in the concentration of calcium ions. Among these photoproteins, aequorin is one of the most well studied (Blinks et al., 1976).

Isolated in the jellyfish Aequoria victoria (Shimomura et al., 1962), aequorin is a Ca²⁺ sensitive photoprotein, i.e., it is modified and/or activated when interacting with Ca²⁺. Said modification and/or activation is detectable especially through non-invasive ways. More particularly, when aequorin interacts with Ca²⁺, after binding with two or three calcium ions, it emits a flash of blue light with a spectrum of maximum wavelength 470 nm. Contrary to a classical luciferase-luciferin reaction, the emission of light does not require exogenous oxygen, and the total amount of light is related to the amount of protein and the concentration of Ca²⁺. Oxygen is molecularly bound and the reconstitution of aequorin occurs, by the action of apoaequorin, a protein with a molecular mass of 21 kDa, and coelenterazine. The emission of photons is caused by a peroxidation reaction in the coelenterazine, after binding with the calcium ions on the aequorin protein. Two hypotheses have been suggested for this process: (i) the binding between aequorin and calcium ions induces the emission of light by a conformational change in the protein, allowing oxygen to react with coelenterazine, and (ii) oxygen plays a role in the binding between coelenterazine and apoaequorin (Shimomura and Johnson, 1978). Aequorin may be recreated in vitro and in vivo by coelenterazine for example by adding it directly into the medium or by administration, and particularly injection, into an organism (Shimomura and Johnson, 1978).

Up to thirty different semi-synthetic aequorins can be produced by replacing the coelenterazine moiety in aequorin with different analogues of coelenterazine (Shimomura, 1995). The different semi-synthetic acquorins show spectral variations, different Ca²⁺ binding affinities, variations in stability, membrane permeability and relative regeneration rates. Measurements of Ca²⁺ concentrations can be undertaken between 100 nM and 1 mM, using different combinations. When native aequorin is reconstituted with native coelenterazine, it has a low affinity for Ca2+ (Kd=10 μM), making it a good sensor in the range of biological Ca²⁺ concentration variations. Although the relationship between light emission and calcium ion concentration may not be linear, a logarithmic relationship between the emission of light and the calcium ion concentration has nonetheless been determined (Johnson and Shimomura, 1978). The fractional rate of aequorin consumption is proportional, in the physiological pCa range, to [Ca2+]. Indeed, a 200-fold increase in the signal to background noise ratio is measured when the Ca²⁺ concentration goes from 10-7M to 10-6M, and by a factor of 1000, from 10-6M to 10-5M (Cobbold and Rink, 1987). Moreover, the kinetics of the signal emission is rapid enough to detect transitory increases in Ca²⁺ ion concentrations. An increase in light intensity with a time constant of 6 msec, under calcium saturation conditions, has been shown (Blinks et al., 1978). Aequorin is thus a photoprotein that is well adapted to measure rapid and elevated increases in Ca²⁺ ions under physiological conditions. Recent studies have investigated the CRET response time of a GFP-aequorin reporter with a linker (Gorokhovatsky et al, 2004). They indicate that the Ca²⁺ triggered bioluminescence reaction of GFP-aequorin exhibits the typical flash-type bioluminescence reaction of aequorin. After addition of Ca²⁺ in a stopped-flow apparatus, light emission begins immediately and reaches a peak within 50 ms. The response kinetics appears to be comparable with the association rate constant indicated for ‘cameleons’ (Miyawaki et al, 1997).

The cloning of the apoaequorin gene by Prasher et al., (1985) and Inouye et al. (1985) has led to the creation of expression vectors, making possible its targeting in a specific cell compartment by fusion with nuclear, cytoplasmic, mitochondrial, endoplasmic reticulum or plasma membrane signal peptides (Kendall et al., 1992; Di Giorgio et al., 1996). In addition, the in vivo expression of the protein makes possible its detection at low levels, leaving the intracellular physiology of calcium undisturbed.

In nature, photoprotein activity is very often linked to a second protein. The most common is the “green fluorescent protein” or GFP. The light emitted in this case is in fact green. The hypothesis of an energy transfer between aequorin and GFP by a radiative mechanism was proposed in the 1960s by Johnson et al., (1962). The blue light emitted by aequorin in the presence of Ca²⁺ is presumably absorbed by GFP and reemitted with a spectrum having a maximum wavelength of 509 nm. Other studies have shown that this transfer of energy occurs through a non-radiative mechanism made possible through the formation of heterotetramer between GFP and aequorin. Morise et al. (1974) have succeeded in visualizing this energy transfer in vitro, by co-adsorption of the two molecules on a DEAE-cellulose membrane. However, these studies indicated that the quantum yield of Ca²⁺-triggered luminescence of aequorin in this condition was 0.23, which coincides with that of aequorin alone (Morise et al, 1974).

GFP, also isolated in the jellyfish Aequoria victoria, was cloned (Prasher et al., 1992). It has been used in different biological systems as a cellular expression and lineage marker (Cubitt et al., 1995). Detecting this protein using classical fluorescence microscopy is relatively easy to do in both living organisms and fixed tissue. In addition, fluorescent emission does not require the addition of a cofactor or coenzyme and depends on an autocatalytic post-translational process. The fluorophore, consisting of nine amino acids, is characterized by the formation of a cycle between serine 65 and glycine 67, which gives rise to an intermediate imidazolidine 5, followed by oxidation of tyrosine 66, transforming it into dehydrotyrosine (Heim et al., 1994). This group is found inside a cylinder composed of 11 β layers, which constitutes an environment that interacts directly with the chromophore (Yang et al., 1996).

Monitoring calcium fluxes in real time could help to understand the development, the plasticity, and the functioning of many organs, such as the central nervous system, the heart, the brain and the liver, and their associated pathologies. In jellyfish, the chemiluminescent, calcium binding, aequorin protein is associated with the green fluorescent protein (GFP), and a green bioluminescent signal is emitted upon Ca²⁺ stimulation. Aequorin alone is difficult to detect on the cellular and subcellular level owing to the weak emission of photons after excitation and makes it extremely difficult to detect in single-cells or with good temporal resolution.

A new marker sensitive to calcium with an apparent higher quantum yield is described in WO01/92300. This marker utilizes Chemiluminescence Resonance Energy Transfer (CRET) between the two molecules. Calcium sensitive bioluminescent reporter genes were constructed by fusing GFP and aequorin resulting in much more light being emitted. Different constructs obtained by recombination of the nucleic acid molecules encoding the GFP linked to aequorin are disclosed in the international application WO 01/92300, which is incorporated herein by reference.

Chemiluminescent and fluorescent activities of these fusion proteins were assessed in mammalian cells. Cystosolic Ca²⁺ increases were imaged at the single cell level with a cooled intensified CCD (coupled charge device) camera. This bifunctional reporter gene allows the investigation of calcium activities in neuronal networks and in specific subcellular compartments in transgenic animals.

This invention utilizes a fusion protein or recombinant protein constructed with aequorin and GFP to increase the quantum yield of Ca²⁺-induced bioluminescence. This activity can not be increased simply by co-expressing GFP with aequorin

Aequorin has a low calcium binding affinity (Kd=10 μM) so it should not have a major effect as a [Ca²⁺]i buffer system nor should it flatten Ca²⁺ gradients. Kinetics of the signal emission is rapid enough to detect transitory increases in Ca²⁺ ion concentration, with a time constant of 6 msec, under calcium saturation conditions. The total amount of light is proportional to the amount of protein and Ca²⁺ concentration. It is therefore possible to calibrate the amount of light emitted at any given time point into a concentration of calcium. Studies have shown that Aequorin alone is extremely difficult to detect at the single-cell and subcellular level due to the weak level of photon emission.

The binding of Ca²⁺ to aequorin, which has three EF-hand structures characteristic of Ca²⁺ binding sites, induces a conformational change resulting in the oxidation of celenterazine via an intramolecular reaction. The coelenteramide produced is in an excited state and blue light (max: 470 nm) is emitted when it returns to its ground state (Shimomura & Johnson, 1978). When GFP is fused to Aequorin by a flexible linker (WO 01/92300), the energy acquired by aequorin after Ca²⁺ binding, is transferred from the activated oxyluciferin to GFP without emission of blue light. The GFP acceptor fluorophore is excited by the oxycoelenterazine through a radiationless energy transfer. The result is the emission of a green shifted light (max, 509 nm) when the excited GFP returns to its ground state.

The GFP-Aequorin of the invention is a dual reporter protein combining properties of Ca²⁺-sensitivity and fluorescence of aequorin and GFP, respectively. The recombinant protein can be detected with classical epifluorescence in living or fixed samples and can be used to monitor Ca²⁺ activities by detection of bioluminescence in living samples. The GFP-Aequorin polypeptide is genetically-encoded and the coding sequence and/or the expressed polypeptide can be localised to specific cellular domains. It can also or alternatively be transferred to organisms by transgenesis without perturbing the function of the photoprotein. This nucleic acid encoding the recombinant polypeptide of the invention can also be expressed under the control of an appropriate transcriptional and/or translational system. “Appropriate” as used herein refers to elements necessary for the transcription and/or the translation of a nucleic acid encoding the recombinant polypeptide of the invention in a given cell type, given tissue, given cellular compartment (such as mitochondria, chloroplast . . . ) or given cellular domains. To achieve said specific expression the nucleic acid is for example recombined under the control of cell-type specific promoters or tissue-type specific promoter, which can enable the measurement of Ca²⁺ signaling in a single-cell type or single tissue-type, within a determined tissue or in whole animals.

Chemiluminescent and fluorescent activities of the GFP-Aequorin protein have been assessed in mammalian cells. Cytosolic Ca²⁺ increases have been previously imaged at the single-cell level with a cooled intensified CCD (coupled charge device) camera (WO 01/92300). Our studies of GFP-Aequorin at the single-cell level demonstrate the sensitivity of this recombinant polypeptide for use as a probe (see results hereafter). GFP-Aequorin does not significantly interfere with local Ca²⁺ signaling due to its low affinity for Ca²⁺. GFP-aequorin is therefore a bioluminescent reporter of intracellular Ca²⁺ activities and can be used to follow dynamic changes in single-cells, tissue slices or living animals. GFP fluorescence is also a valuable reporter of gene expression and marker of cellular localization. Moreover, bioluminescent molecules do not require the input of radiative energy as they utilize chemical energy to produce light. Hence, there is virtually no background in the signal.

In contrast, fluorescent dyes cannot be localised exclusively to subcellular domains. Chameleons on the other hand are genetically targetable. These reporters generally have a low signal-to-noise ratio and long-term imaging is difficult due to phototoxicity and problems associated with photobleaching. This limits the use of these probes for visualising dynamic changes over prolonged periods, for example in studies of learning and memory, development and circadian rhythms. Fluorescence requires radiative energy, which results in photobleaching, phototoxicity, autofluorescence and a high background signal. An external light source is necessary in order to excite fluorescent molecules. Excitation light will be absorbed when passing through tissue to excite fluorescent molecules. Similarly, the same will occur when the emission light is detected through tissue. For the moment, in vivo non-invasive whole animal imaging is largely restricted to bioluminescent reporters.

### Other Related Techniques:

Electrophysiological recording is restricted to the cell-soma or large dendritic regions that are accessible with a micropipette.

Yuste et al. describes the use of fluorescent indicators to detect activation of a “follower” neuron that relates to the optical detection of a connection between two neurons or between a plurality of neurons (Yuste et al, 2003). This approach, however, suffers from the disadvantage that these fluorescent indicators are useful only for short-term or time lapse imaging applications and because they can not be genetically-targeted. Specifically; (1) Non-selective staining and the problem of dye leakage from cells after a short period at physiological temperature (2) The requirement of light excitation restricts long-term dynamic imaging due to photobleaching and photodynamic damage caused to living (or fixed) dissociated cell culture or tissue samples. (3) Imaging localised Ca²⁺ dynamics or high Ca²⁺ concentration microdomains with fluorescent dyes is difficult and below the limits of spatial resolution offered by light microscopy techniques.

Voltage-sensitive dyes are discussed as a useful technique for monitoring “multineuronal activity in an intact central nervous system” (Wu et al, 1998). These probes are fluorescent and are therefore subject to the same limitations as discussed for Ca²⁺ sensitive fluorescent dyes (see Knopfel et al, 2003 for review). A genetically encodable form has also been developed (Siegel & Isacoff, 1997), but has a low signal-to-noise ratio.

‘Cameleons’ are a class of genetically encoded Ca²⁺ sensitive fluorescent probes consisting of two GFP's covalently linked by a calmodulin binding sequence. Chameleons generally have a low signal-to-noise ratio and long-term imaging is difficult due to phototoxicity and problems associated with photobleaching. Targeting of this probe has been made to the mitochondrial matrix (Fillipin et al, 2003), to the lumen of the endoplasmic reticulum (Varadi & Rutter, 2002) and to the surface of large dense core secretory vesicles via fusion with a transmembrane protein known as phogrin (Emmanouilidou et al, 1999).

This invention describes a novel approach using combined fluorescence/bioluminescence imaging of single-cell type, including neurons and neuronal populations, to detect calcium signalling microdomains associated with synaptic transmission and to visualise in real-time the calcium dynamics in single-cell type, such as in neurons and neuronal networks as well as other organs and tissues.

In particular, this invention provides a recombinant polypeptide useful for detection of Ca²⁺ microdomains. The recombinant polypeptide comprises a bioluminescent polypeptide, optionally fused to a peptide or a protein capable of targeting to a subcellular domain. In one embodiment of the invention, the bioluminescent polypeptide comprises a chemiluminescent peptide that binds calcium ion, and a fluorescent peptide. In another example, the recombinant polypeptide consists of said chemiluminescent peptide binding calcium ions and said fluorescent peptide. The recombinant polypeptide may also consist of a chemiluminescent peptide, a fluorescent peptide and a linker, and optionally is further fused to a peptide or a protein capable of targeting to a subcellular domain. In a particular embodiment, the recombinant polypeptide consists of a chemiluminescent peptide, a fluorescent peptide and a peptide or a protein capable of targeting to a subcellular domain. Another example is a recombinant polypeptide consisting of a chemiluminescent peptide, a fluorescent peptide, a linker and a peptide or a protein capable of targeting to a subcellular domain

Targeting of GFP-Aequorin (GA) to subcellular compartments or cellular microdomains is possible by fusion with a peptide signal or a peptide or protein of interest.

According to a particular embodiment, this invention describes the targeting and use of the dual fluorescent/bioluminescent recombinant protein (GFP-Aequorin), to detect calcium signalling in cellular compartments and particularly in calcium microdomains associated with synaptic transmission. This invention also describes the use of these recombinant polypeptides for the ‘real-time’ optical detection of calcium dynamics in single cell or population of cells, such as single neurons and in neuronal populations. Although these studies describe the use of this recombinant polypeptide in neurons, they are intended also to highlight the sensitivity and other important characteristics offered by this reporter polypeptide. The use of this recombinant polypeptide is certainly not restricted to use in neurons. GFP-Aequorin has tremendous utility also in other cell types but certainly in most animal cells, plants, bacteria and also yeast, specifically any living system whereby calcium signalling is important.

An example of a chemiluminescent peptide is Aequorin or a mutant of aequorin. In a preferred embodiment, the mutant Aequorin has a different, and preferably lower, affinity for calcium ion, such as the mutant aequorin Asp407→Ala. In another example, it can have a higher affinity, when the h-coelenterazine analogue is used to regenerate the aequorin protein.

An example of a fluorescent peptide is green fluorescent protein (GFP), a variant of GFP or a mutant of GFP. Such a variant has the feature to emit photons at a different wavelength. Examples of such GFP variants are CFP (cyan fluorescent protein), YFP (yellow fluorescent protein) and RFP (red fluorescent protein). Other examples, such as mStrawberry and mCherry, are described in Shaner et al., Nature Biotechnology, 22:1567-1572 (2004), Shaner et al., Nature Methods, 2:905-909 (2005), and Giepmans et al., Science, 312:217-224 (2006).

A mutant or a variant of a chemiluminescent peptide or a fluorescent peptide is defined herein as a sequence having substitutions, deletions or additions according to the reference sequence. The amino acid substitutions can be conservative, semi-conservative or non-conservative.

Therefore, a particular recombinant polypeptide consists of Aequorin and GFP, especially of fusion polypeptide GFP-aequorin, with or without linker between them. Are included in the scope of the invention, fusions between Cyan fluorescent protein and aequorin (CFP-aequorin), between yellow fluorescent protein and aequorin (YFP-aequorin), between red fluorescent protein and aequorin (RFP) or triple fusions including any of these combinations (e.g. RFP-YFP-aequorin) as well as mutant or variant of the original GFP-aequorin, being a red-shifted version of GFP-aequorin or having mutations improving the brightness, the stability and/or the maturation of the reporter protein.

This invention also provides a recombinant polypeptide which consists of Aequorin, GFP and a linker, especially a peptidic linker. In a particular recombinant polypeptide, said chemiluminescent peptide is aequorin, the fluorescent peptide is GFP, and the aequorin and GFP are linked by a peptidic linker allowing Chemiluminescence Resonance Energy Transfer (CRET). A peptidic linker allowing CRET comprises or consists preferably of 4-63 amino acids and especially of 14-50 amino acids. In a particular embodiment, such a peptidic sequence comprises or consists of the sequence [Gly-Gly-Ser-Gly-Ser-Gly-Gly-Gln-Ser]ₙ with n is 1-5, and preferably n is 1 or n is 5.

In another particular embodiment of the recombinant polypeptide of the invention, the peptide or protein which is capable of targeting to a subcellular domain is selected from Synaptogamin, PSD95, subunit VIII of cytochrome C oxidase, and immunoglobulin heavy chain or a fragment thereof such as the N-terminal fragment.

This invention also provides a recombinant polynucleotide encoding the polypeptide of the invention.

In addition, this invention provides a vector comprising the polynucleotide of the invention and a host cell containing said recombinant polynucleotide or said vector. The cell can be, for example, a eukaryotic cell, or a prokaryotic cell, such as an animal cell, a plant cell, a bacteria, or a yeast.

The invention concerns a method for optical detection of the dynamics of Ca²⁺ in a biological system, said method comprising monitoring the photons emitted by a recombinant Ca²⁺-sensitive polypeptide of the invention, which comprises or consists of a chemiluminescent protein fused, or linked to a fluorescent protein, present in said biological system. Any polypeptide described in this application can be used in the carrying out of said detecting method. This method is useful for the optical detection of intracellular Ca²⁺ signaling or of the propagation of Ca²⁺ signal to detect communication from one cell to another.

Said method can be carried out for the monitoring of photons emission in different biological systems: in vitro in a cell or group of cells, in vivo in a animal or plant expressing said recombinant polypeptide of the invention or ex vivo in a tissue or group of cells from a transgenic animal or plant.

Said method comprises, prior to the monitoring of the emission of photons, the administration of said recombinant polypeptide or of a polynucleotide encoding said recombinant polypeptide into the biological system. In whole animal system, the recombinant polypeptide or the nucleic acid encoding it (or corresponding vector) is administrated preferably by intravenous, intraperitoneal or intramuscular injection. The administration of the nucleic acid encoding the recombinant polypeptide of the invention can be carried out by any appropriate means especially by recombinant vectors, in particular by recombinant viral vectors. In a particular embodiment of the invention, transgenic non-human animal or transgenic plant are provided, which have especially been transformed by the nucleic acid encoding the recombinant polypeptide. The transformation can be transient or definitive. In this case, the recombinant polypeptide of the invention is expressed from the modified genome of the plant or animal.

When expressed from the genome of a transgenic plant or animal, the expression and/or localization of said recombinant polypeptide may be restricted to a specific tissue, a single-cell type (such as neural, heart or liver cell) or a cellular compartment or domain (such as mitochondria or chloroplast).

Said method can also comprise, prior to the monitoring of the emission of photons, the administration of a molecule allowing the activation of the bioluminescent and/or fluorescent proteins. In the GFP-aequorin reporter protein, the method comprises the administration of coelenterazine in the biological system, in conditions and concentrations enabling the activation of the aequorin. Aequorin/coelenterazine systems have been disclosed in the art (Shimomura, 1991).

In a particular embodiment, this invention provides a method for detecting or quantifying Ca²⁺ at the subcellular level. The method comprises expressing in vivo a recombinant polypeptide of the invention encoded by a polynucleotide of the invention in a host cell especially in a non-human animal, and visualizing the presence of Ca²⁺. Optionally, the Ca²⁺ can be semi-quantified. In a preferred embodiment, the detection is a so-called “real-time” detection.

The invention also provides a method for the identification of physiological and/or pathological processes comprising optionally the characterization of the development morphology or functioning of a group of cells, a tissue, a cell or a cellular compartment or domain by GFP fluorescence detection, and the characterization of dynamics of Ca²⁺ in said group of cells, said tissue, said cell or said cellular compartment or domain by the method of optical detection of the invention.

The invention further relates to a method for the identification of physiological and/or pathological processes that may involve variations of calcium fluxes or signaling out of known normal ranges, wherein the method comprises the optical detection of the dynamics of Ca²⁺ in accordance with the present application. Alternatively said optical detection of the dynamics of Ca²⁺ can rather be included as a part of a protocol for the identification of such processes in particular in order to perform diagnosis or monitoring, for example monitoring of a therapeutic response.

In addition, this invention provides a transgenic non-human animal or plant, comprising a host cell of the invention.

This invention also provides a transgenic non-human animal, usable in the above-method of optical detection, expressing a genetically-encoded recombinant polypeptide of the invention as described above. In a particular embodiment, the recombinant polypeptide is encoded by a polynucleotide, optionally under the control of an appropriate transcriptional and translational system, inserted in the genome of said transgenic animal. This non-human animal can be a vertebrate and particularly mammals, such as primates or rodents. In a particular embodiment, this non-human animal is rat, rabbit or mouse.

This invention also concerns a method for producing a transgenic non-human animal of the invention comprising:

transferring a DNA construct into embryonic stem cells of a non-human animal, wherein said DNA construct comprises or consists of a sequence so-called transgene encoding a recombinant polypeptide sensitive to calcium concentration, said recombinant polypeptide comprising or consisting of a chemiluminescent protein linked to a fluorescent protein, and wherein said transgene is under the control of a promoter and optionally of conditional expression sequences,

selecting positive clones, wherein said DNA construct is inserted in the genome of said embryonic stem cells,

injecting said positive clones into blastocytes and recovering chimeric blastocytes,

breeding said chimeric blastocytes to obtain a non-human transgenic animal.

In a particular embodiment, wherein the expression of the recombinant polypeptide is conditional, the following method for producing a transgenic non-human animal can be used:

transferring a DNA construct into embryonic stem cells of a non-human animal, wherein said DNA construct comprises or consists of a sequence so-called transgene encoding a recombinant polypeptide sensitive to calcium concentration, said recombinant polypeptide comprising or consisting of a chemiluminescent protein linked to a fluorescent protein, and wherein said transgene is under the control of a promoter and optionally of conditional expression sequences,

selecting positive clones, wherein said DNA construct is inserted in the genome of said embryonic stem cells,

injecting said positive clones into blastocytes and recovering chimeric blastocytes,

breeding said chimeric blastocytes to obtain a first non-human transgenic animal,

crossing said resulting first non-human transgenic animal with an animal expressing an endonuclease, acting on said conditional expression sequences, in the tissues or cells in which expression of said recombinant polypeptide is needed, and

recovering a transgenic non-human animal expressing said recombinant polypeptide in specific tissue or cells.

“Conditional” as used herein means that the recombinant protein sensitive to calcium concentration of the invention is expressed at a chosen time throughout the development of the non-human transgenic animal. Therefore, in a particular embodiment, the recombinant protein is expressed when a recombinase catalyzes the recombination of conditional expression sequence, and for example when the enzyme Cre catalyses the recombination of the Lox recognition sites.

The expression of the recombinase or endonuclease, such as Cre, can be both spatially and temporally regulated according to the promoter located upstream of the nucleic acid encoding said recombinase. Said promoter can be a cell-specific promoter allowing the expression of the recombinase for example in liver, heart or brain cells.

In a particular embodiment, the expression of the recombinase may be activated by natural or synthetic molecules. Therefore, a ligand-dependent chimeric Cre recombinase, such as CreERT or CreERT2 recombinases, can be used. It consists of Cre fused to modified hormone binding domains of the estrogen receptor. The CreERT recombinases are inactive, but can be activated by the synthetic estrogen receptor ligand tamoxifen, therefore allowing for external temporal control of Cre activity. Indeed, by combining tissue-specific expression of a CreERT recombinase with its tamoxifen-dependent activity, the recombination of conditional expression sites, such as Lox sites, can be controlled both spatially and temporally by administration of tamoxifen to the animal.

The invention also relates to the offspring of the transgenic non-human animals of the invention. These offspring may be obtained by crossing a transgenic animal of the invention with mutant animals or models of disease.

In a further embodiment of the invention, there is provided a method for screening molecules of interest to assay their capacity in modulating Ca²⁺ transients, wherein said method comprises:
a) detecting the dynamics of Ca²⁺ by the method of optical detection of the invention in a transgenic animal expressing a recombinant polypeptide sensitive to calcium concentration,
b) administering or expressing the molecule of interest into said transgenic animal,
c) repeating step a), and
d) comparing the location, the dynamics, and optionally the quantity, of Ca²⁺ before and after injection, wherein a variation in the location, the dynamics and/or the quantity of Ca²⁺ is indicative of the capability of the molecule to modulate Ca²⁺ transients.

This invention also provides a recombinant peptidic composition capable of being expressed in vivo in a non-human animal by a polynucleotide encoding GFP, aequorin, and a peptide or a protein capable of targeting said recombinant peptide into a cellular domain. The peptidic composition is involved in the visualization or the quantification of Ca²⁺ changes in a cell, group of cells, subcellular domain or tissue of interest.

This invention provides means to genetically target the bioluminescent reporter, GFP-Aequorin, to different microdomains including those important in synaptic transmission. GFP-Aequorin has an excellent signal-to-noise ratio and can be targeted to proteins or to cellular compartments without perturbing photoprotein function. The invention therefore, enables ‘real-time’ visualisation of localised Ca²⁺ dynamics at molecular, cellular, tissue and whole animal level or in dissociated cell cultures, excised tissues, acute and organotypic cultures or living animals.

The reporters of the invention enable selective detection of subcellular or high Ca²⁺ concentration microdomains. The genetically encoded bioluminescent Ca²⁺ reporter, GFP-Aequorin, can therefore be used to optically detect synaptic transmission and to facilitate the mapping of functional neuronal circuits in the mammalian nervous system.

Whole-animal bioluminescence imaging represents a very important non-invasive strategy for monitoring biological processes in the living intact animal. To date, applications describing in vivo imaging of cellular activity with use of bioluminescent reporters have been almost exclusively undertaken with the luciferin-luciferase system from the firefly. The approach takes advantage of the luciferase reporter system for internally generated light linked to specific biological processes. Bioluminescent reactions usually involve the oxidation of an organic substrate (luciferin or chromophore). Light is generated when cells expressing the luciferase are combined with the substrate, luciferin (peak at 560 nm). Both ATP and O₂ are required for the light reaction to take place. As these reporters have been developed to emit light shifted in the red at longer wavelengths, the light produced is less absorbed by tissue, making this technology ideal for following tumour progression or infection.

The aequorin based system offers alternative applications to the luciferase based reporter system for BLI. Light is generated in the presence of Ca²⁺ and the substrate, coelenterazine. In contrast to the luciferin-luciferase system, the Ca²⁺ dependent light emission of GFP-aequorin (peak at 515 nm) does not require exogenous O₂. In contrast, molecular O₂ is tightly bound and the luminescence reaction can therefore take place in the complete absence of air. Therefore, the bioluminescence kinetics of the photoprotein is not influenced by the oxygen concentration. The second feature of aequorin is that the light intensity can be increased up to 1 million fold or more on the addition of calcium. The coelenterazine-GFP-aequorin system therefore enables a specific analysis of Ca²⁺ activities and can be more suitable than the firefly system as a reporter, because it does not require the co-factors ATP and Mg2+. Given that the luciferase reaction results in the emission of red light, it is more suited for deep tissue analysis and therefore ideal for following infectious process or for following tumor progression. However the aequorin based system, can be utilized to monitor Ca²⁺-dependent biological processes with spatial and fine temporal resolution at more superficial tissue sites (analysis of Ca²⁺ signal in the mammalian cortex, in skeletal muscles or in skin). With the development of new instrumentation, the GFP-aequorin-coelenterazine system could be imaged in deep tissue layers in the same manner as the luciferase-luciferin system. Whole animal in-vivo imaging of the GFP-aequorin-coelenterazine system can therefore allow to investigate dynamic biological processes in living animal models of human biology and disease.

The inventors have developed transgenic mice expressing different GFP-aequorin reporter polypeptides. The expression of the nucleic acid encoding this recombinant polypeptide of the invention is driven by appropriate transcriptional and/or translational elements, which preferentially localize upstream of said nucleic acid, but may also localize downstream.

These reporter mice offer several advantages over other non gene-based or gene-based reporters, because they can report non-invasively multiple activities in living samples and can be realized in-vivo. A preferred embodiment is a transgenic mouse expressing mitochondrially targeted GFP-aequorin, where mitochondrial Ca²⁺ activities can be monitored by bioluminescence imaging and GFP fluorescence can be visualized to localize reporter expression and to study specific morphological characteristics. Mitochondrial function is a useful biosensor of cellular activities, particularly for following pathological processes.

Transgenic animals expressing GFP-aequorin reporters could be used for developing diagnostics or for screening new drugs or for evaluating therapeutic response in preclinical trials. Transgenic animals expressing GFP-aequorin reporters could also be crossed with transgenic animal models of disease in order to study pathological processes. For example, transgenic mice expressing mitochondrially targeted GFP-aequorin in all cells or selected cell types can be crossed with transgenic mouse models of Alzheimer's disease to assess pathological processes, develop new diagnostics or evaluate therapeutic response in preclinical trials. Excised tissues and/or dissociated cells derived from transgenic animals expressing GFP-aequorin reporters, can also be applied in high-throughput screening assays for discovery of new drugs, or for assessing activities of existing drugs or to evaluate therapeutic response in preclinical trials or to develop diagnostics.

A problem to be solved in the construction of a transgenic animal is the expression of the inserted transgene, that must be sufficiently efficient for the production of the encoding protein. This may be carried out by the insertion of the polynucleotide encoding the recombinant protein sensitive to calcium concentration or the corresponding DNA construct in a transcriptionally active region of the genome of the animal to transform, or by reconstituting a particularly favourable environment ensuring a correct gene expression, such as a reconstituted HPRT locus. The insertion is carried out having recourse to any technique known from the skilled person in the art, and particularly by homologous recombination.

Another problem to face is the specific expression of this recombinant protein in tissue, organs or cell types. This can be achieved by using recombination system comprising conditional expression sequences and corresponding recombinases such as endonuclease. Particular conditional expression sequences are Lox sites and the corresponding endonuclease is Cre, that is preferentially expressed under the control of a cell- or tissue-specific promoter.

In vivo imaging of GFP-aequorin according to the invention has been shown to be non-invasive, and can be used to monitor physiological processes, pharmacokinetics, pathological and other aspects of biomolecular processes occurring functions in the living animal. Detection of Ca²⁺ could be used to assess and monitor many different cellular signaling pathways in the context of studying different pathologies, drug effects and physiological processes. Detection of Ca²⁺ is a useful diagnostic in many pathological conditions, including, cancer, infection processes, neuropathological diseases, muscle disorders (e.g. muscular dystrophy), for treatment and diagnosis of cardiovascular disorders. GFP-aequorin technology could also be applied to all mammals and other animals, e.g. worms (e.g. Nematodes), fish (e.g. Zebrafish), frogs (Xenopus sp.) and flies (Drosophila sp.).

For instance, calcium imaging offers an alternative approach for monitoring liver, heart or brain activity. For example, Ca²⁺ signals are linked to the electrical activity of neurons and to the propagation of activity via glial to glial, glial to neuron, neuron to neuron or neuron to glial cell signaling (e.g. chemical and gap-junctional coupling). Furthermore, Ca²⁺ is involved in many intracellular signalling pathways. Spatiotemporal profiles of Ca²⁺ in cellular microdomains regulate the activation of key signalling pathways. Hence, by genetically localizing a reporter to nanodomains or microdomains, cellular events can be monitored in real-time at the molecular level, even when there is very little spatial resolution, as it is the case in whole animal imaging. Described hereafter are multi-functional reporter mice for in-vivo, ex-vivo and in-vitro imaging in research and development applications.

### Material and Methods

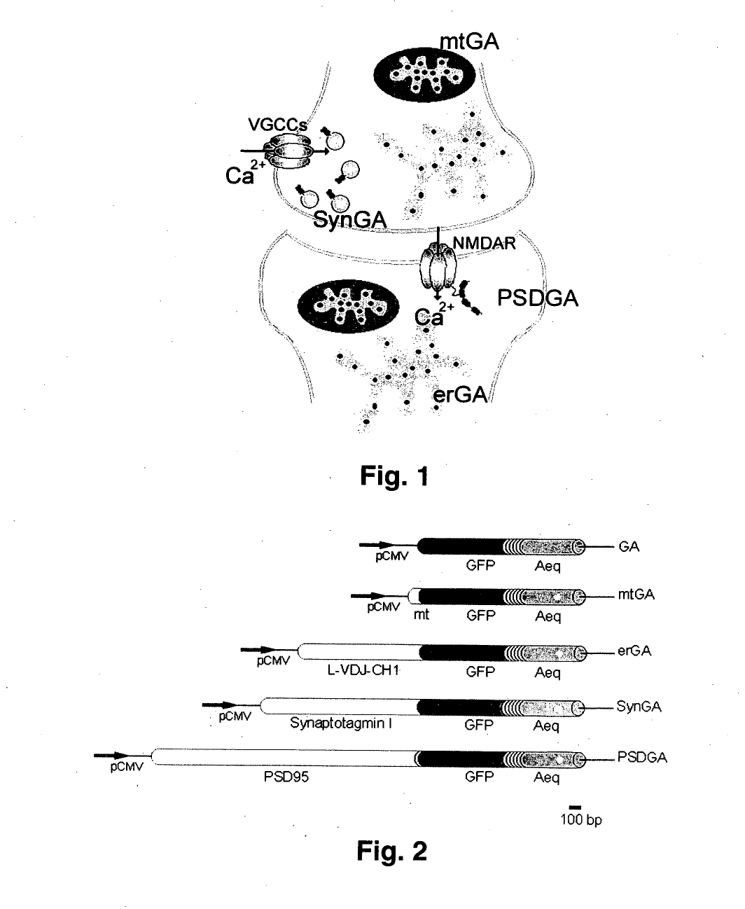

### Construction of Targeted Vectors (FIGS. 1 and 2)

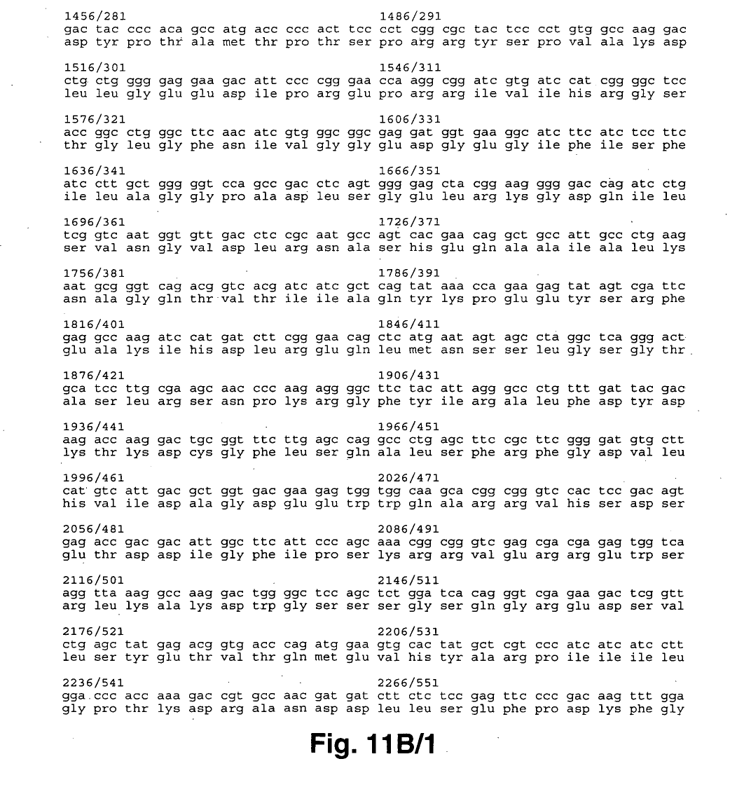

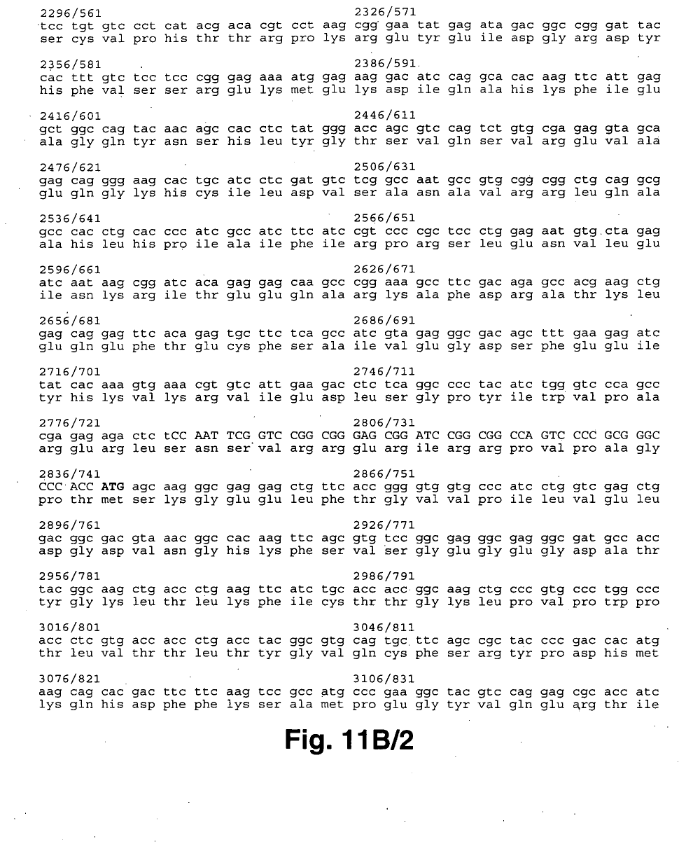

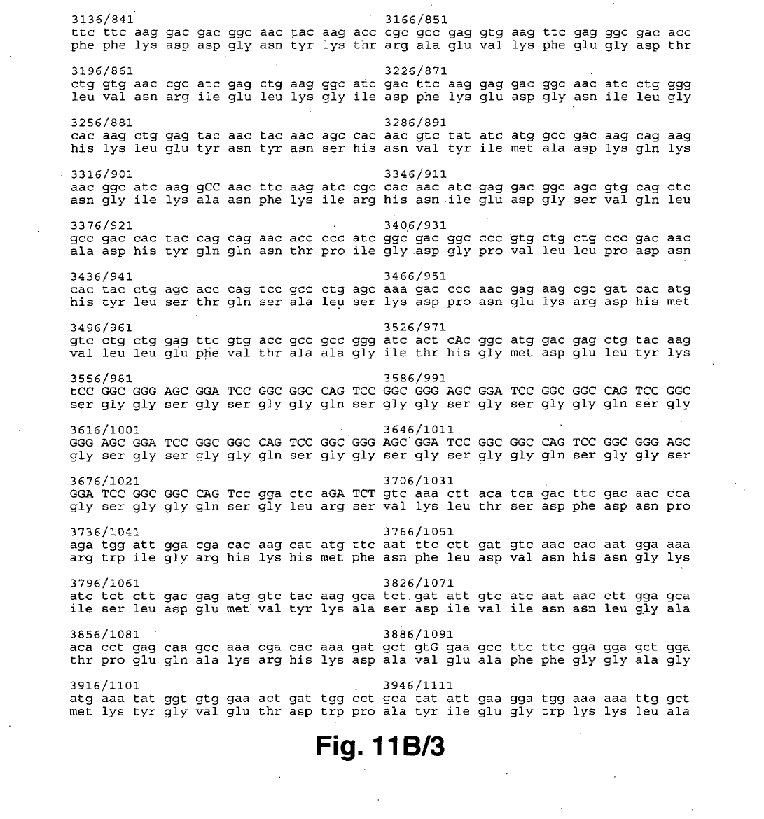

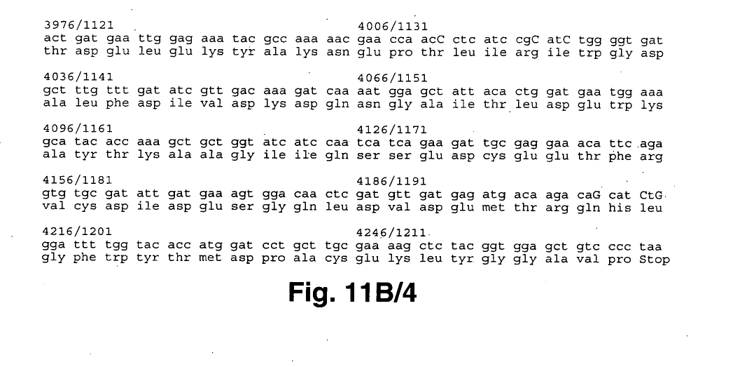

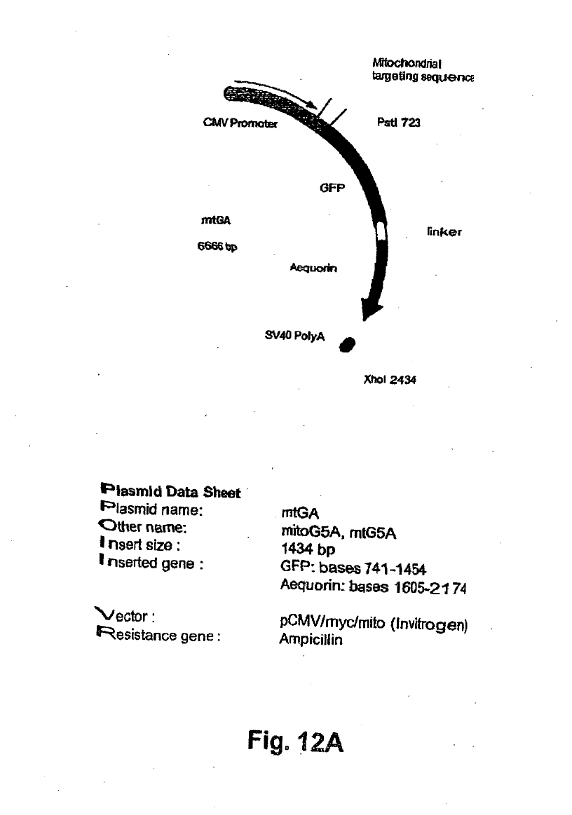

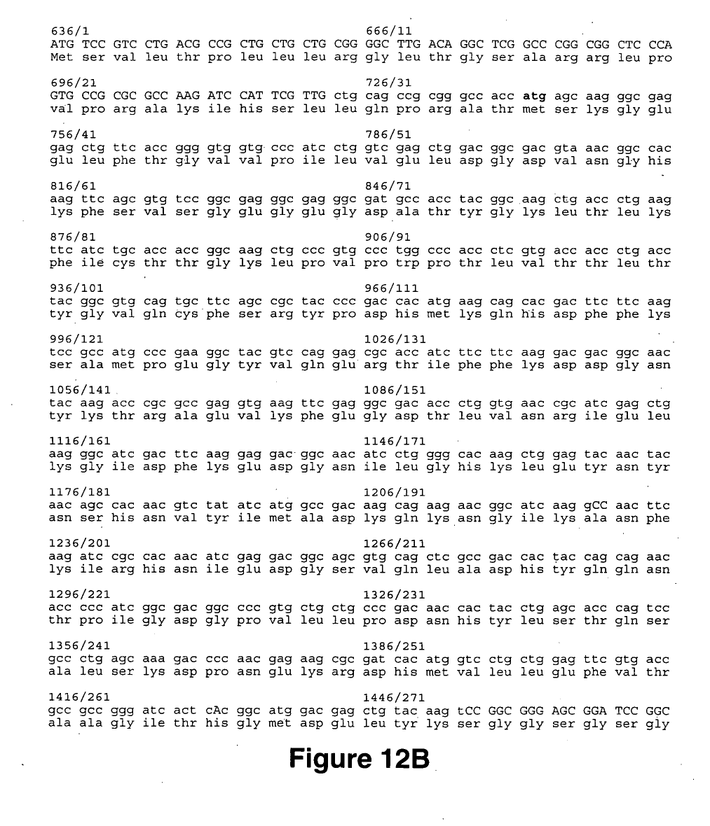

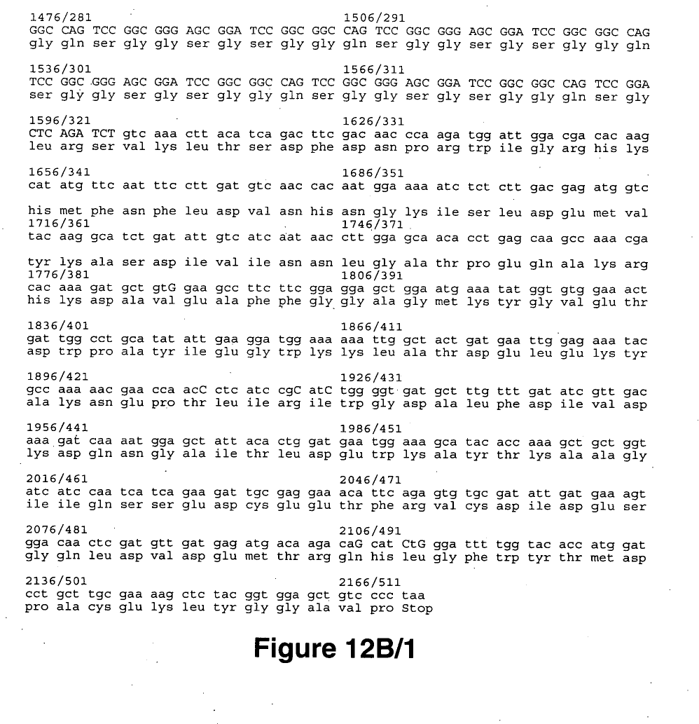

GA represents non-targeted GFP-Aequorin denoted G5A containing a 5-repeat flexible linker between the two proteins. Construction of GFP-Aequorin (GA) and Synaptotagmin-G5A (SynGA) has been described previously (WO 01/92300). For targeting of the GFP-Aequorin chimaera to a post-synaptic domain, we created a fusion between the N-terminal region of PSD95 and GA. In this construction the full length of the PSD95 gene (FIG. 12) was cloned HindIII/EcoRI into pGA (C.N.C.M. I-2507, deposited on Jun. 22, 2000) to give the plasmid, PSDGA. A flexible linker was then added between PSD95 and the start of GFP, composed of the following sequence 5′ A ATT CGG TCC GGC GGG AGC GGA TCC GGC GGC CAG TCC CCG C ′3 (FIG. 11).

The GFP-Aequorin chimaera (GA) has been targeted to the mitochondrial matrix by cloning the reporter gene into the Pst I/xho I sites of the vector containing the cleavable targeting sequence of subunit VIII of cytochrome c oxidase (pShooter, Invitrogen) to give the plasmid mtGA (FIG. 12). GA was also targeted to the ER lumen, by cloning in frame to the N-terminal region of the immunoglobulin (IgG) heavy chain gene, which consists of the leader sequence, VDJ and the CH1 domains. The coding sequence for the N-terminal region of the IgG heavy gene was removed with Nhe I and Hind III from the plasmid erAEQmut, which was kindly provided by Dr J Alvarez (Universidad de Valladolid, Spain). The gene insert was ligated in frame to the N-terminal of the G5A gene in the pEGFP-C1 vector (Clontech), to give the plasmid erGA.

A mutation (Asp-407→Ala) to reduce the Ca²⁺ binding affinity of the photoprotein (Kendall et al, 1992), was generated by PCR in each of the targeted GA constructs to give the plasmids, mtGAmut, erGAmut, SynGAmut and PSDGAmut. All constructs are under the control of the human cytomegalovirus promoter (pCMV). All sequences have been verified by DNA sequencing.

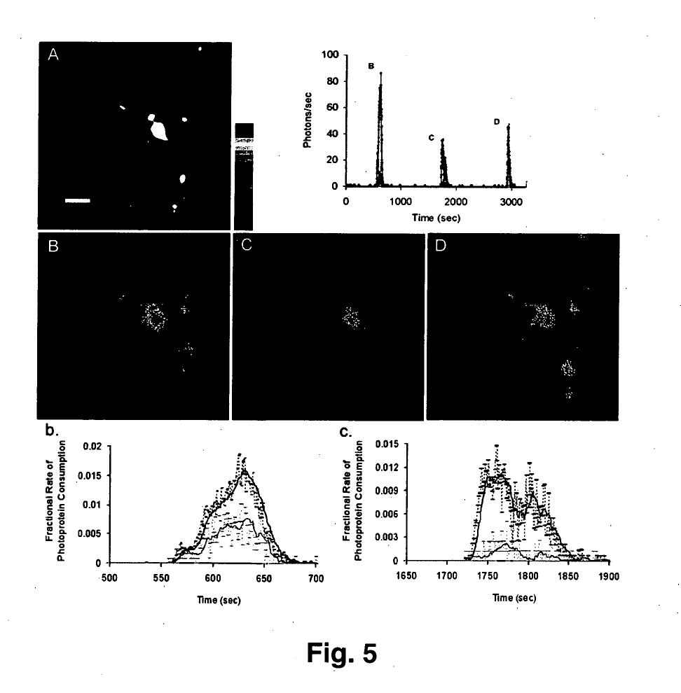

### Single-Cell Bioluminescence Studies (FIGS. 5-9)

Cultures were plated on to glass-bottomed dishes and mounted to a stage adapter on a fully automated inverted microscope mounted in a black-box. GFP-Aequorin was reconstituted with 2.5-5 μM coelenterazine for 30 minutes at 37° C. Incubation of cells with coelenterazine that had been transfected with SynGA was undertaken at room temperature to reduce the consumption of the photoprotein during the reconstitution process. Cells were perfused with tyrodes buffer. Prior to bath application of NMDA, cells were perfused for 1 minute without Mg²⁺. All recordings were made at room temperature (22-25° C.). Cells having a cell soma diameter between 10-15 μm, which were phase bright without granular appearance, were selected for measurements. Cells transfected with SynGA and reconstituted with wildtype coelenterazine, regularly displayed a low level of bioluminescence activity at resting state. Activity appeared to be homogenous and more evident in the cell soma region. This suggests that GA targeted in this fashion, is within a domain endowed with a high concentration of Ca²⁺. On two occasions, cells transfected with PSDGA, showed spontaneous activity that was localised to dendritic regions.

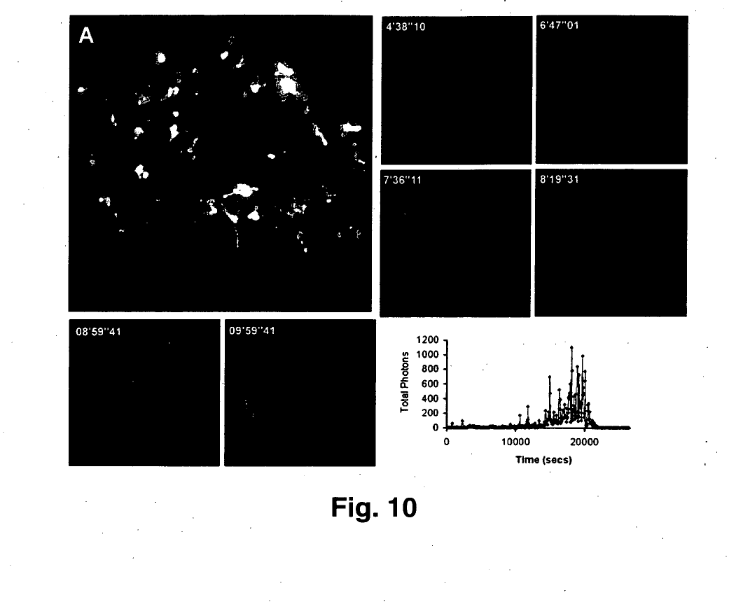

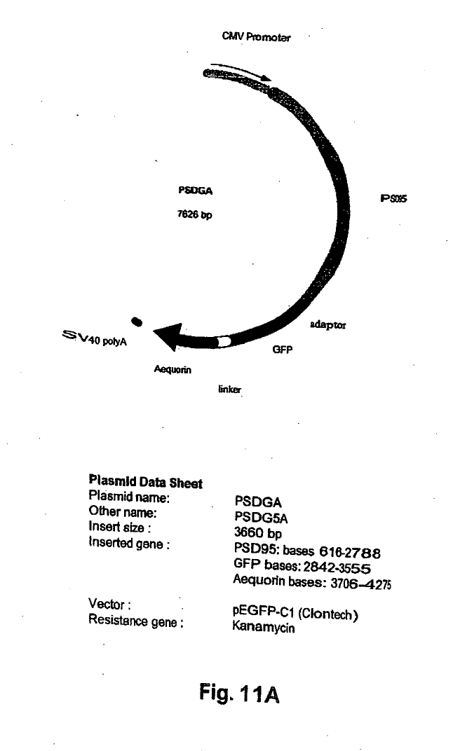

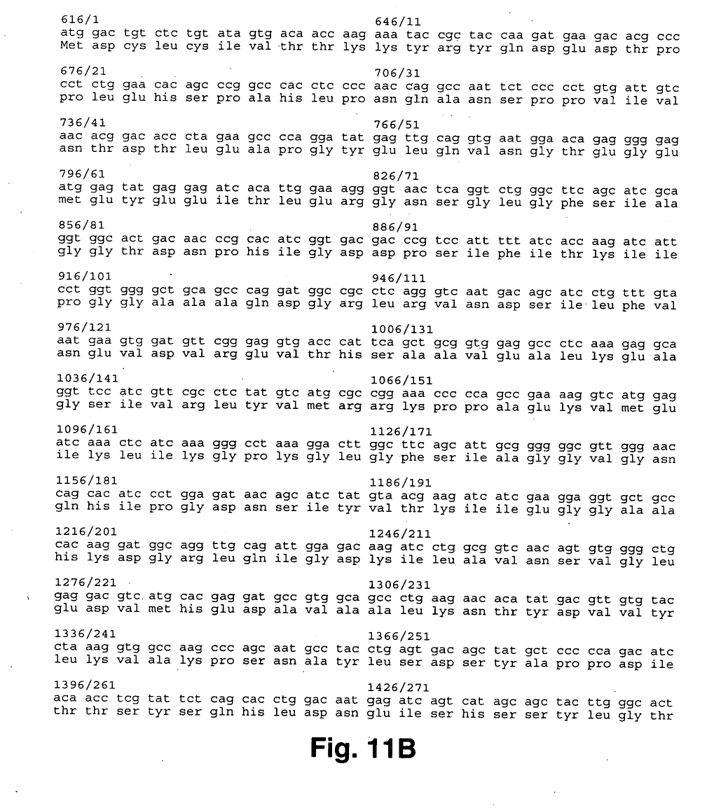

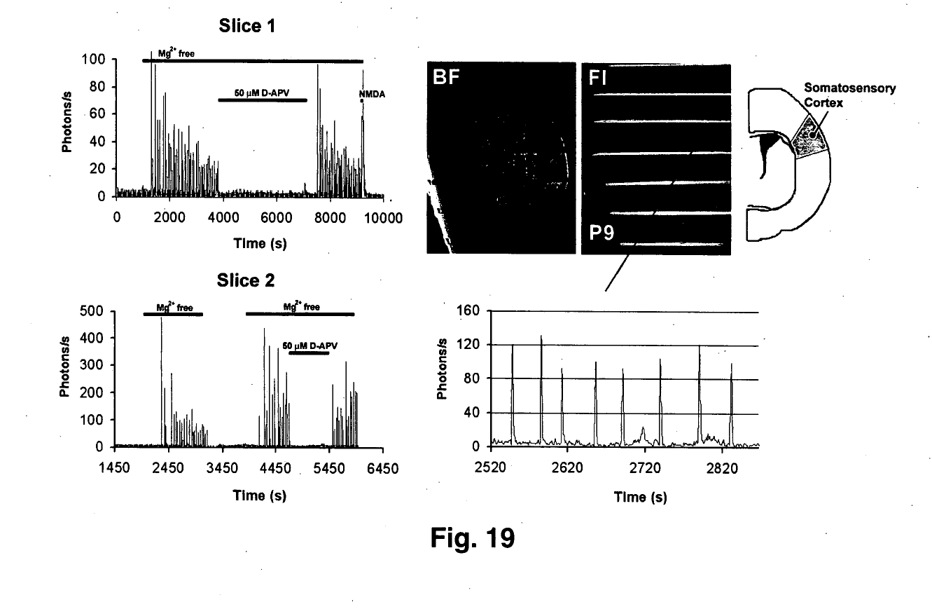

### Imaging Ca²⁺ Dynamics in Organotypic Slice Cultures (FIGS. 10 and 19)

Organotypic hippocampal slices were prepared from 4-5 day old mice pups. Briefly, brains were rapidly removed in ice-cold Hanks buffer and sliced into 200 μm slices with a tissue chopper. Hippocampal slices were identified using a stereo microscope and transferred into sterile transwell collagen coated chambers (12 mm diameter, 3.0 μm pore size). Slices were maintained in Neurobasal medium supplemented with B27 at 37° C., in a humidified atmosphere containing 5% CO₂. Slices were infected at day 9 with the Adenovirus-GFP-Aequorin vector and maintained in culture for a further 4 or 5 days before imaging. At this stage, slices appeared healthy and individual cells could be clearly distinguished by GFP fluorescence. After incubation with coelenterazine, slices could be maintained on an inverted microscope at room temperature for up to 9 hours at which time cell death became apparent, indicated by large increases in bioluminescence activity and loss of cellular fluorescence. Because light excitation is not required to detect the Ca²⁺ reporter, long-term imaging can be performed without causing photodynamic damage. Imaging over long periods is continuous. It is not necessary to select an integration period. Background is extremely low, less than 1 photon/sec in a 256×256 pixel region (665.6×665.6 μm). In some experiments, coronal 400-450 μm slices from the somatosensory cortex of transgenic mice were cut using a vibratome and placed in culture for 4-5 days before imaging. Slices were perfused with ACSF bubbled with 95% O₂ and 5% CO₂. Following 1 hour of incubation, slices were perfused with Mg2+ free ACSF. Recordings were made at room temperature (25-28° C.). Drugs were added via the perfusate.

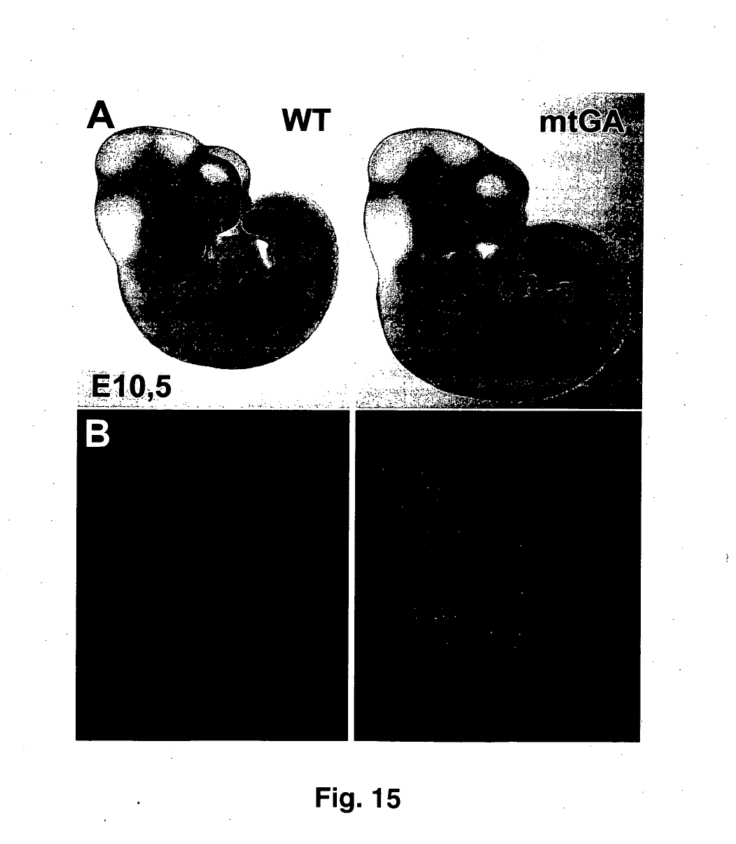

### Construction of Transgenic Animals (FIGS. 15-23)

We have genetically engineered new reporter molecules, whereby GA is targeted to sub-cellular domains in transgenic mice. The GA transgene can be expressed in any cell type and/or at any stage of development. Expression has been made conditional by using a Lox-stop-Lox sequence immediately after the strong promoter, β-Actin (CAG). Selection of the expressing cells is made by using an appropriate endonuclease Cre, driven by specific promoters. The time at which the transcription will be started will be realized by injecting tamoxifen when the gene Cre-ER^{T2} will be used. Finally, to have the possibility to express in any cell, the transcription unit has been introduced by homologous recombination in ES cells in the reconstituted HPRT locus (X chromosome) to minimize the influence of the integration site on the level of expression. In the experiments shown here, a PGK Cre transgenic mouse that activates the GA transgene in very early embryo was used.

Recombinant viruses containing CRE that are under the regulation of a cell specific promoter can also be used. Mice have been constructed by injection of genetically modified ES cells into blastocysts.

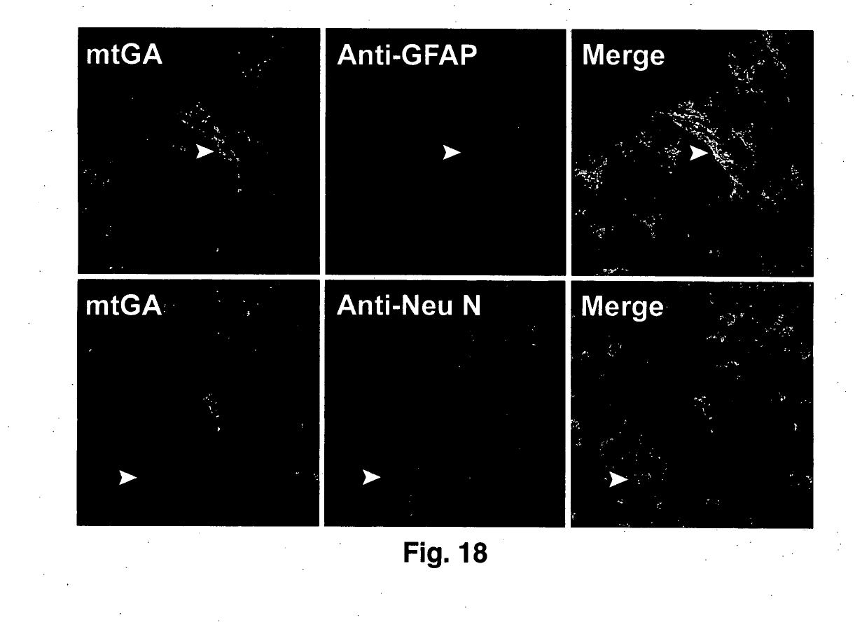

Immunolocalisation Studies with mtGA (FIG. 18)

Cortical neurons or brain slices expressing the mitochondrially targeted GFP-aequorin reporter were fixed for 20 min in 4% formaldehyde in PBS at RT. After washing with PBS, cell membranes were permeabilised with PBS containing 0.1% Triton-X 100 and BSA. Cells were then incubated at RT for 1-2 hours with primary antibodies. Targeting was compared to anti-cytochrome c (1:500; BD Biosciences Pharmingen, CA, USA) and MitoTracker® Red CMXRos (200 nM; Molecular Probes Inc.). The binding of antibodies was determined after incubation for 1 hour in secondary antibodies conjugated to Alexa Fluor®546 (Molecular Probes, Inc.). After washing, cells were mounted on slides in Fluoromount and visualized by confocal analysis. Images were acquired on an Axiovert 200M laser scanning confocal microscope (Zeiss LSM-510; version 3.2) through a 63×/1.4 NA, oil immersion objective using LP560 and BP505-550 filters. The pinhole aperture was set at 98 Tm and images were digitized at a 8-bit resolution into a 512×512 array.

### Combined Fluorescence/Bioluminescence Imaging (FIGS. 5-7, 8-10, 13, 14 and 19)

The fluorescence/bioluminescence wide field microscopy system was custom built by ScienceWares, Inc. The system includes a fully automated inverted microscope (200M, Zeiss Germany) and is housed in a light-tight dark box. Mechanical shutters control illumination from both halogen and HBO arc lamps, which are mounted outside of the box and connected via fiber optic cables to the microscope. Low level light emission (photon rate <100 kHz), was collected using an Image Photon Detector (IPD 3, Photek Ltd.) connected to the baseport of the microscope, which assigns an X, Y coordinate and time point for each detected photon (Miller et al., 1994). The system is fully controlled by the data acquisition software, which also converts single photon events into an image that can be superimposed with brightfield or fluorescence images made by a connected CCD camera to the C-port (Coolsnap HQ, Roper Scientific). Any Ca²⁺ activity that is visualised can therefore be analysed in greater detail by selecting a region of interest and exporting photon data. After an experiment has been completed, the recorded movie file can be replayed and data can be extracted according to the users needs. The IPD can provide sub-milisecond time resolution and integration times are not required to be specified for the acquisition. The system we are using has very low background levels of photon counts, <1 photon/second in a 256×256 pixel region.

Calibration of bioluminescence measurements into intracellular Ca²⁺ values in living cells can be performed by in vitro calibration. Intracellular [Ca²⁺] measurements were made by determining the fractional rate of photoprotein consumption. For in vitro calibration, Neuro2A cells were transiently transfected with the different constructs. After 48 hours, cells were washed with PBS and harvested using a cell scraper. The cell suspension was transferred to a 1.5 ml Eppendorf tube and incubated in an aequorin reconstitution buffer containing 10 mM mercaptoethanol, 5 mM EGTA, and with either the native (wt), n or h coelenterazine 5 μM in PBS, at 4° C. for 2 hours. After 2 hours, cells were washed and resuspended in a hypo-osmotic buffer containing 20 mM Tris/HCl, 10 mM EGTA and 5 mM mercaptoethanol in dH₂0 and protease inhibitor, EDTA free (Roche Diagnostics). Cell membranes were further lysed by three freeze-thaw cycles, followed by passing the suspension through a 26 GA needle. 10 μl aliquots of cell lysates containing the reporter protein were dispensed into the wells of white opaque 96-well plates, which contained EGTA buffered solutions having known concentrations of CaCl₂ (Molecular Probes, Inc.). Free Ca²⁺ was calculated using the WEBMAXC program (www.stanford.edu/˜cpatton/webmaxc.html) (Bers et al., 1994). Luminescence was directly measured using a 96-well plate reader (Mithras, Berthold Tech. Germany). Light was recorded for 10 s, with 100 ms integration after injection of the cell lysate. After 10 s, 100 μL of a 1 M CaCl₂ solution was injected into the same well and recording was continued until light returned to basal levels and all of the photoprotein had been consumed (Lmax). Light emission is expressed as the fractional rate of photoprotein consumption, which is the ratio between the emission of light (L, s⁻¹) from that time point (defined [Ca²⁺]) and the integral of total light emission from that point until full exhaustion of the photoprotein (Lmax) (saturating Ca²⁺). Experiments were undertaken at 25-28° C.

### Electrical Stimulation and Viral Transfection

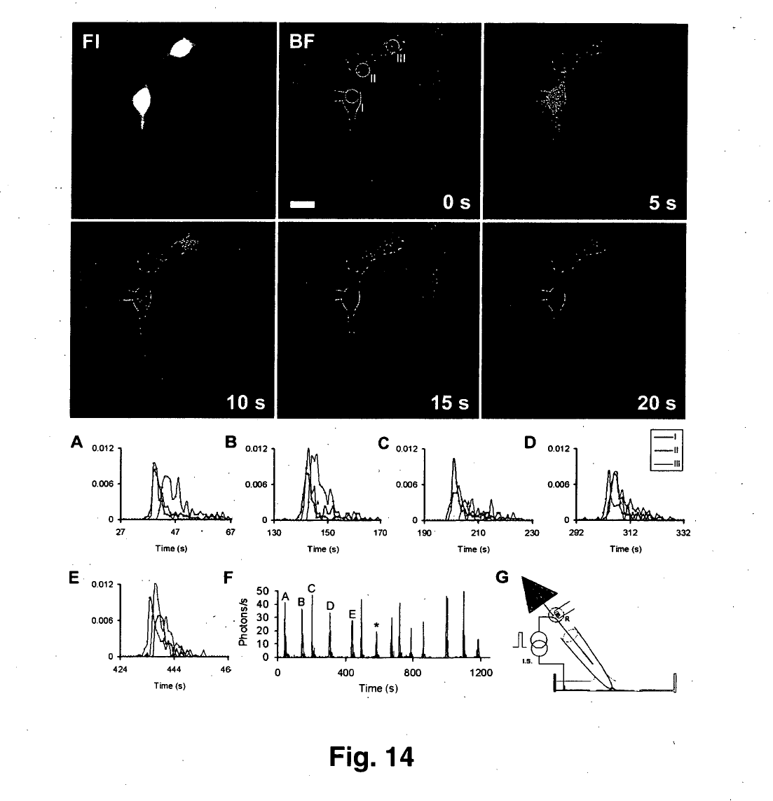

In some experiments, electrical pulses were delivered to the cell under study via a classical patch pipette (5-10 MΩ), pulled from borosilicate glass (World Precision Instruments, Florida, USA). An electrically operated relay system made within the laboratory, allowed for measurement of the pipette resistance between stimulations delivered by an isolated stimulator (DS2A, Digitimer Ltd, England) (see FIG. 14). The propagation rate of Ca²⁺ waves was calculated by taking the time point corresponding to the half maximum of light emitted after stimulation and dividing by the distance measured between the center of the two regions analysed.

### Whole Animal Bioluminescence Detection

Native coelenterazine (4 μg/g of body weight; Interchim France) was introduced by an intra-peritoneal injection into P1-P4 mice. Imaging of mice began 1-1.5 hours after injection of the substrate, coelenterazine. An IVIS Imaging System 100 Series, which allows real-time imaging to monitor and record cellular activity within a living organism was utilized in these studies to detect local Ca²⁺ changes at the whole animal level. The system features a cooled back-thinned, back illuminated CCD camera, inside a light-tight, low background imaging chamber. A greyscale surface image of mice was initially acquired by using a 10 cm field of view, 0.2 s exposure time, a binning resolution factor of 2, 16 f/stop (aperture) and an open filter. Bioluminescence images were acquired immediately after the greyscale image. Acquisition times for bioluminescence images ranged from 1-5 seconds, binning 8 & 16, field of view 10 cm; f/stop 1. Relative intensities of transmitted light from in vivo bioluminescence were represented as a pseudocolor image ranging from violet (least intense) to red (most intense). Corresponding grayscale photographs and color luciferase images were superimposed with LivingImage (Xenogen) and Igor (Wavemetrics, Lake Oswego, Oreg.) image analysis software.

### Results

Result 1: Targeting of GA to Subcellular Domains.

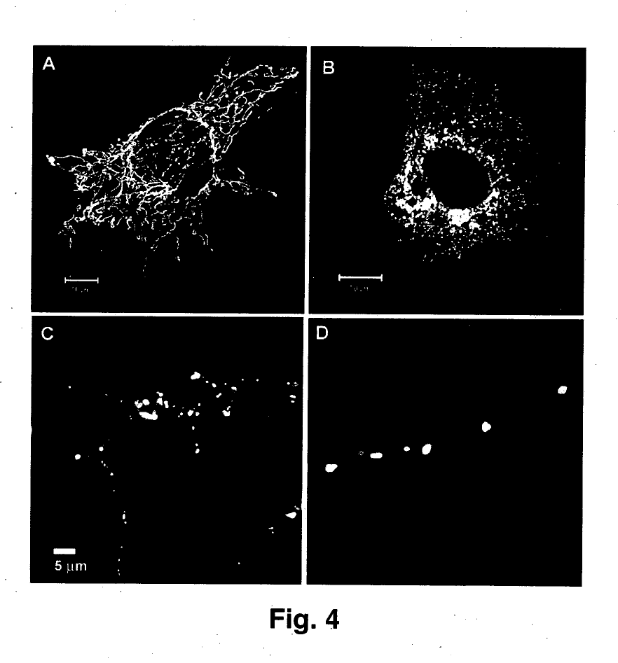

Different GA reporters were constructed by fusion to a signal peptide or protein of interest with the aim to direct expression into specialized subcellular compartments (FIGS. 1 & 2). GA was targeted to domains that are important in synaptic transmission: mitochondrial matrix, endoplasmic reticulum, synaptic vesicles and the post-synaptic density. Confocal analysis shows expected expression patterns of the GA reporters after fusion to the signal peptide of cytochrome c for targeting to the mitochondrial matrix (mtGA), to IgG heavy chain for targeting to the lumen of the ER (erGA), to synaptotagmin I protein for targeting to the cytosolic side of the synaptic vesicle membrane (SynGA) or to PSD-95 protein for targeting to the postsynaptic density (PSDGA) (FIG. 4) (see Christopherson et al., 2003; Conroy et al., 2003).

Result 2: GA Reports Ca²⁺ Concentrations with Single-Cell Resolution.

We began these studies with the non-targeted GA, which distributes homogenously in neurons (FIG. 5). After reconstitution of GA with h coelenterazine (a high affinity version of the luciferin), stimulation with NMDA (100 μM) and KCl (90 mM) produced a robust signal in cortical neurons (FIGS. 5B & C). This is the first time that Ca²⁺ responses in small mammalian neurons have been directly visualised at the single and subcellular level with a bioluminescent reporter. Application of digitonin and high Ca²⁺ at the end of the experiment indicates that there was still sufficient photoprotein remaining (FIG. 5D). High Ca²⁺ and digitonin were also added at the end of the experiment to measure the total available GFP-aequorin (Lmax) for normalizing the data (see definition of Lmax in the methods section).

Result 3: Optical Detection of GFP-Aequorin Targeted to a Synaptic Protein Associated with Calcium Signaling.

Microdomains of High Ca²⁺ are Detected with Targeted GFP-Aequorin Reporters After Stimulation of Cortical Neurons.

### EXAMPLE 1

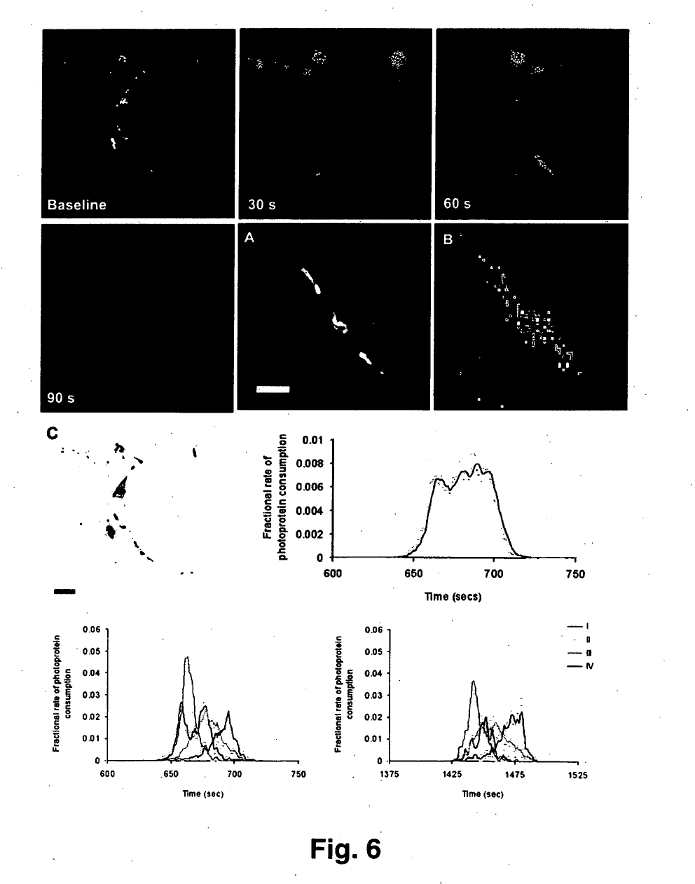

Differences in the kinetic properties of Ca²⁺ responses can be detected subcellularly when GA is targeted to compartments, such as in the mitochondrial matrix (FIG. 6). A representative example is shown where NMDA application caused Ca²⁺ responses in defined cellular locations with different temporal profiles. FIGS. 6A & B illustrates that, despite the low levels of light emission and moderate spatial resolution compared with conventional fluorescence, we can analyse the Ca²⁺ response in specific areas. When reporters are subcellularly targeted, we can still detect a signal that is up to a 1000 fold higher than the background (15×15 pixel region, each pixel=0.65 μm). The graphical data derived from localized regions shown in the graphs demonstrates the diversity in the spatiotemporal properties of mitochondrial Ca²⁺ changes.

### EXAMPLE 2

Optical detection of Ca²⁺ induced bioluminescence in neurons using GFP-Aequorin targeted to the calcium sensor synaptic vesicle transmembrane protein, synaptotagmin I. See FIGS. 1, 2 4D & 7. Synaptotagmin I is a low-affinity Ca²⁺ sensor believed to be involved in the regulation of rapid exocytosis events (Davis et al, 1999). Previous studies show that Synaptotagmin I is “tuned” to respond to Ca²⁺ concentrations (21-74 μM that trigger synaptic vesicle membrane fusion (threshold >20 μM half-maximal rates at 194 μM).) We have constructed a low-affinity version of GFP-Aequorin targeted to synaptic vesicles by fusion to Synaptotagmin I, known as SynGAmut. By using the low-affinity version of the Ca²⁺ reporter, which only detects high calcium concentration domains, it should be possible to optically probe neuronal exocytosis in a specific manner. For example, SynGAmut could allow specific detection of vesicles located in close proximity to voltage-gated Ca²⁺ channels, which are docked for neurotransmitter release (FIG. 4D). These vesicles would be located close enough to the mouth of a channel and therefore within a high Ca²⁺ concentration domain, which is believed to be necessary to drive vesicle exocytosis that facilitates synaptic transmission. Given that the reporter has a lower affinity for Ca²⁺, vesicles that are not docked for release would be sufficiently far enough away not to be detected.

### EXAMPLE 3

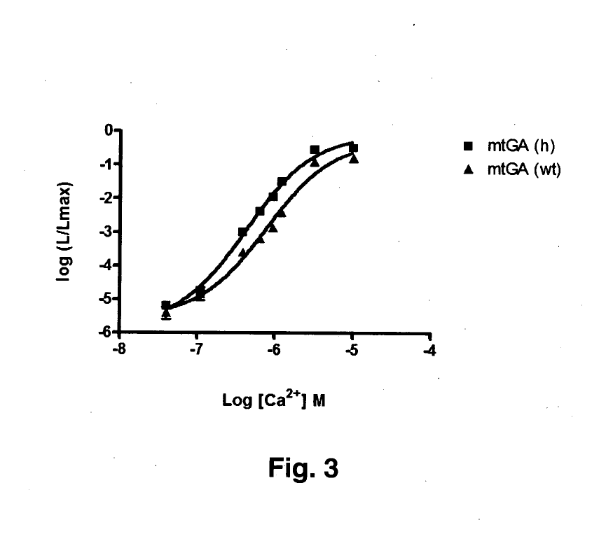

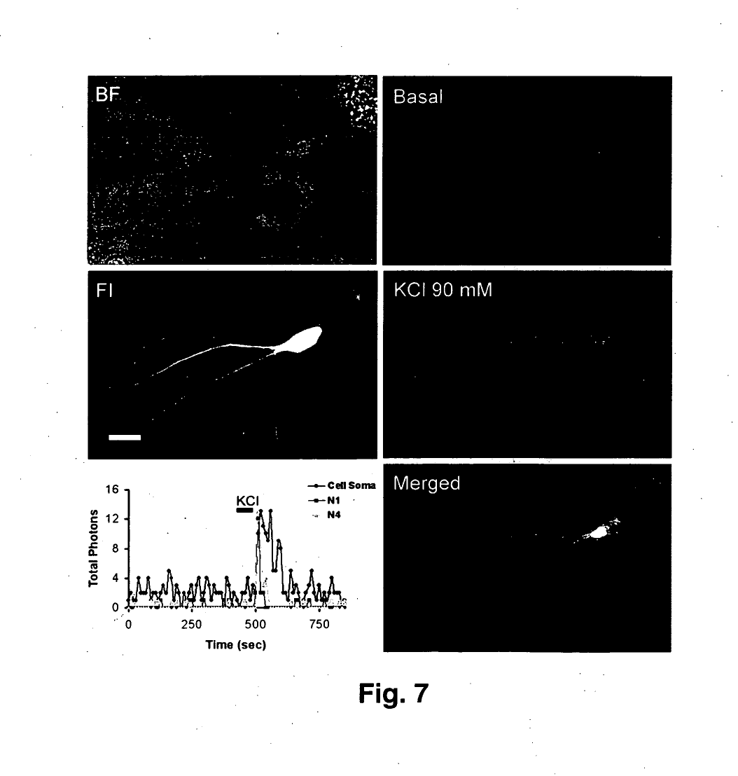

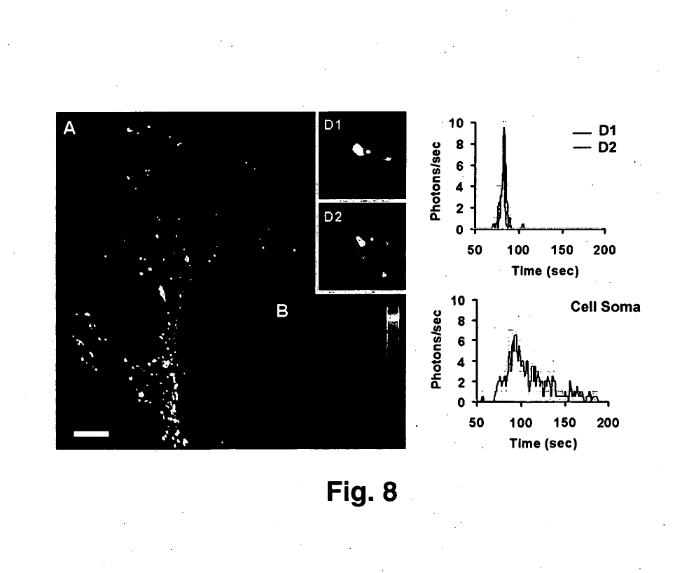

Ca²⁺ induced bioluminescence could also be visualized with subcellular resolution in cortical neurons expressing PSDGA and mtGA after NMDA application (FIGS. 7 & 8). In contrast to GA, application of NMDA to neurons transfected with PSDGA, produced Ca²⁺ transients with faster kinetics and larger amplitudes (see FIG. 8 for a representative example of 3 experiments). Distinct differences in the Ca²⁺ dynamics in the dendrites (FIGS. 8D1 & D2) versus the cell soma were observed (FIG. 8, cell soma). In particular, the dendritic regions analysed exhibited a faster rate of rise with a rapid decay in the Ca²⁺ response. The rapid rate of decay suggests that the photoprotein was completely consumed, rendering the temporal dynamics and amplitude of the Ca²⁺ response to be artifactual. In addition, there was no further available photoprotein remaining, suggesting that the concentration of Ca²⁺ in the domain where PSDGA was targeted to, would have been very high (refer to FIG. 3 for Ca²⁺ binding curve).

### EXAMPLE 4

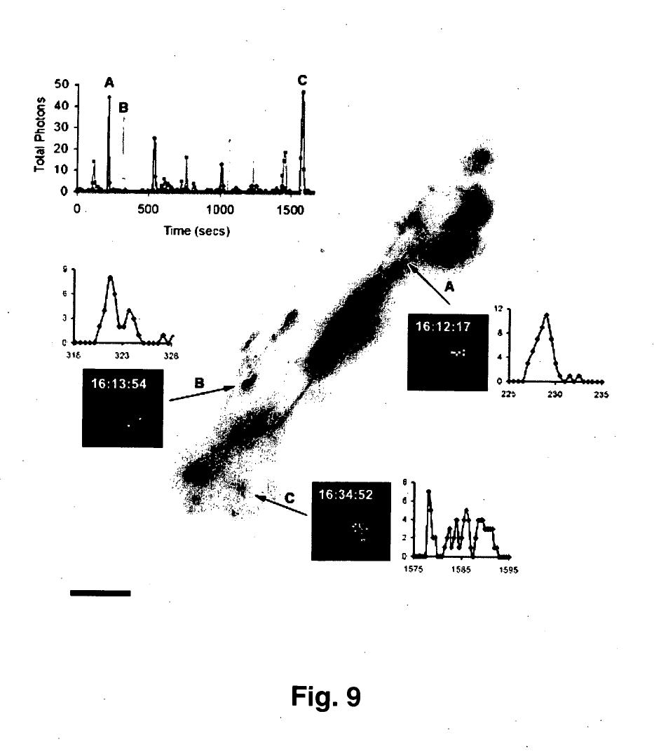

Transient transfection of cortical neurons with PSDGA directs localized expression of GA to dendritic structures (FIGS. 8 & 9). The localized targeting of GA when it is fused to PSD-95 enabled us to observe a specific type of activity in cortical neurons that were kept in basal conditions. In contrast to SynGA where the rate of light emission was constant, we observed random and non-synchronized Ca²⁺-transients over a very low background that were spatially localized in some experiments. In at least two experiments (a representative example is shown in FIG. 9), the calcium transients occurred relatively frequently and were localized to dendritic regions (See FIGS. 9A, C & B).

### EXAMPLE 5

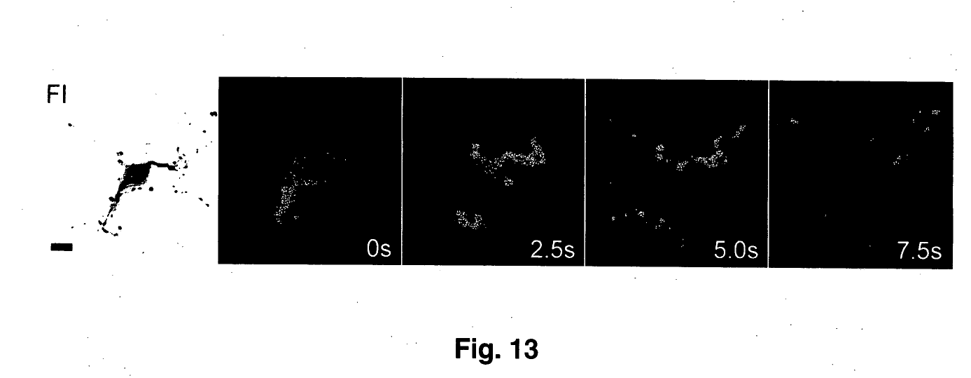

### Progation of Ca²⁺ Intracellularly in a Cortical Neuron Transfected with PSDGA (FIG. 13)

Result 4: Use of Genetically Targeted GFP-Aequorin for ‘Real-Time’ Visualisation of Calcium Dynamics in Neuronal Populations for Mapping Neural Connectivity.

We next examined the use of these reporters for following cell-cell communication in cultured neurons. We also electrically stimulated hippocampal neurons expressing the GA reporter to visualize the propagation of Ca²⁺ activity and cellular communication within simple neural networks. In these experiments, we used a replication defective adenoviral vector coding for GA (Ad5-GA) to transfect dissociated hippocampal neurons. FIG. 14FI shows at least 2 cells expressing the GA reporter. Application of a short electrical pulse to the somatic region of the cell labelled I (FIG. 14BF), results in the propagation of a Ca²⁺ wave to the neighbouring cell labelled III. Analysis of Ca²⁺ responses in three regions, suggests that the propagation of Ca²⁺ was variable between each region. From region I to II the wave was calculated to travel at a rate of 60 μm/s. In contrast, it was calculated to travel at a rate of 10 μm/s 410 from region II to III. A total of 6 successive stimuli (one single pulse approx. every 2 mins) were applied (the first 5 of them are graphically represented), after which spontaneous occurring oscillations appeared (FIG. 14A-F). Each Ca²⁺“spike” displayed a rapid rate of rise followed by a slow decline. Significant photoprotein activity was also still remaining (determined by mechanical rupture of the cell membrane) after recording these Ca²⁺ transients for approximately 45 minutes.

Spontaneous activities recorded in organotypic slices infected with a replication defective adenoviral vector coding for GA (Ad5-GA) (FIG. 10). Long-term recordings (for up to 8 hours) can be undertaken when GA is expressed in tissue slices, such as organotypic slices from the cortex. In general, we find that photoprotein consumption and the level of sensitivity for detecting variations in Ca²⁺ is relevant to the amount of reporter expressed, to the localization of the reporter and to the type of coelenterazine analogues used (Shimomura, 1997; Shimomura et al, 1993). This can vary from application to application, from cell to cell and needs to be optimized in each case, much the same, as it needs to be for fluorescent probes.

Result 5: Construction of a Transgenic Animal Expressing GFP-Aequorin to a Specific Cell-Type, Subcellular Compartment or Cellular Microdomain to Study Calcium Dynamics in Whole Animal Studies.

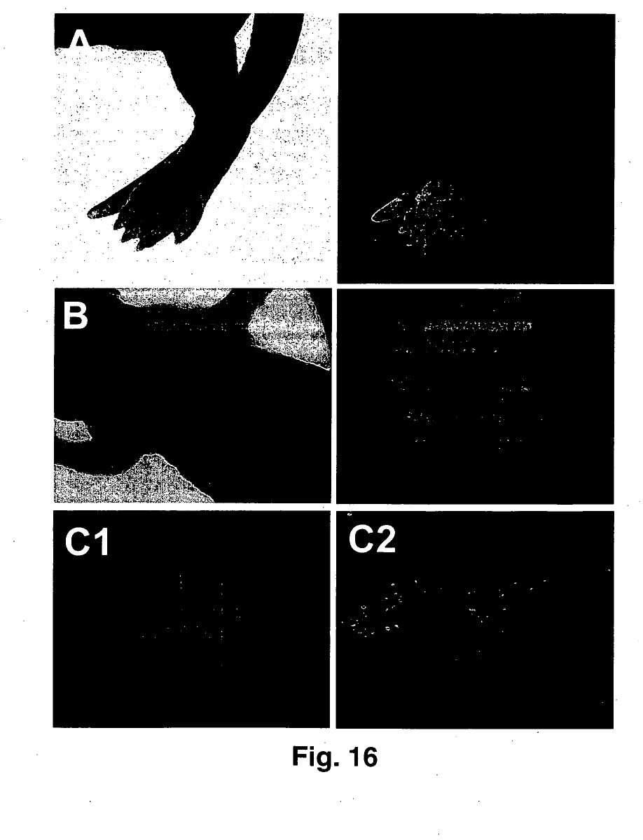

Transgenic animals have been constructed, which express targeted GFP-aequorin reporters. The GA transgene can be expressed in any cell type and/or at any stage of development. Expression has been made conditional by using a Lox-stop-Lox sequence immediately after the strong promoter, β-Actin (CAG). Selection of the expressing cells is made by using an appropriate endonuclease Cre, driven by specific promoters. To have the possibility to express in any cell, the transcription unit has been introduced by homologous recombination in ES cells in the reconstituted HPRT locus to minimize the influence of the integration site on the level of expression. Recombinant viruses containing CRE that are under the regulation of a cell specific promoter can also be used. In the example shown here for a transgenic mouse constructed with GFP-aequorin targeted to the mitochondrial matrix, activation of transgene expression has been induced in all cells and from the beginning of development by crossing these mice with a PGK-CRE mouse. Fluorescence imaging of whole embryos and neonatal mice reveals that the mtGA transgene is expressed in all cell types (FIGS. 15, 17, 18 and 19). The reporter protein is also well targeted to the mitochondrial matrix (FIGS. 16C2 & 18). Some advantage of GFP-aequorin is that it is not an endogenous protein normally expressed by mammalian cells and it has little interference with Ca²⁺ signaling because of its low binding affinity. Accordingly, we do not find evidence of a abnormal phenotype in transgenic lines obtained with GFP-aequorin reporters.

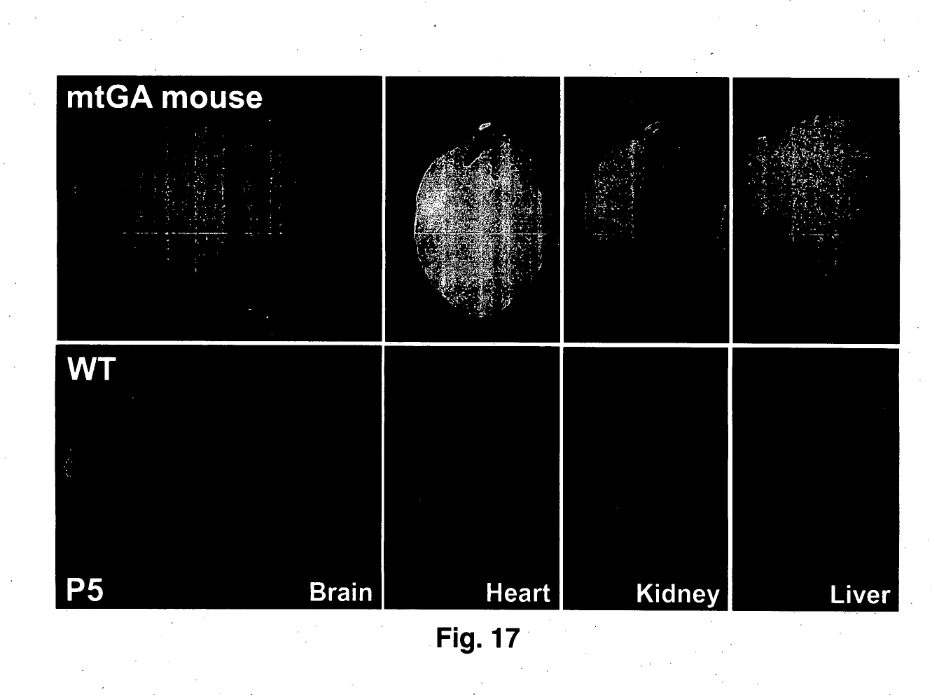

Confocal analysis shows expected expression patterns of the GA reporters after fusion to the signal peptide of cytochrome c for targeting to the mitochondrial matrix (mtGA) (FIG. 16C2). GFP fluorescence images of major organs indicate strong levels of reporter expression in the major organs (FIG. 17). Higher levels of expression are apparent in the heart and liver. In the brain, expression of the transgene is evident in both glial cells and neurons after comparison to antibodies against GFAP and NeuN, respectively (FIG. 19). These results show that the mtGA transgene is well targeted and expressed in all cells of the brain when transgenic mtGA reporter mice are crossed with PGK-CRE mice.

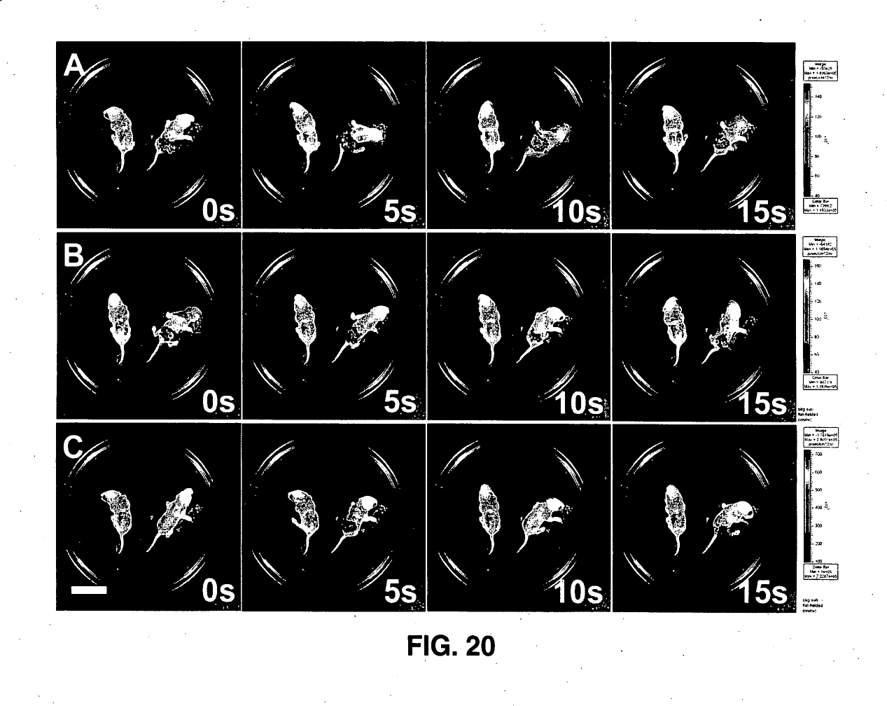

Analysis of luminescence activities indicates that the GFP-aequorin protein is functional in respect to detection of Ca²⁺. We have obtained preliminary data using our transgenic mice, showing that we can detect mitochondrial Ca²⁺ oscillations in organotypic slices from the neocortex (FIG. 20). The Ca²⁺ transients occurred synchronously across a large-scale area at a rate of once every 15-45 secs. As we are using bioluminescence to detect Ca²⁺ activities, slice imaging can be undertaken for periods of up to 8 hours in real-time. Our results show that the GFP-aequorin reporter provides an excellent signal-to-noise ratio for detecting the Ca²⁺ transients in brain slices from transgenic animals.

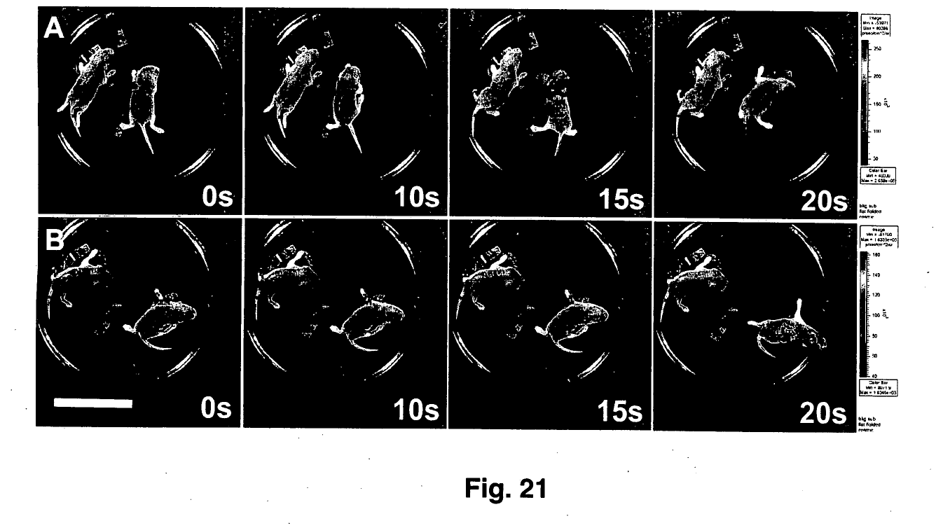

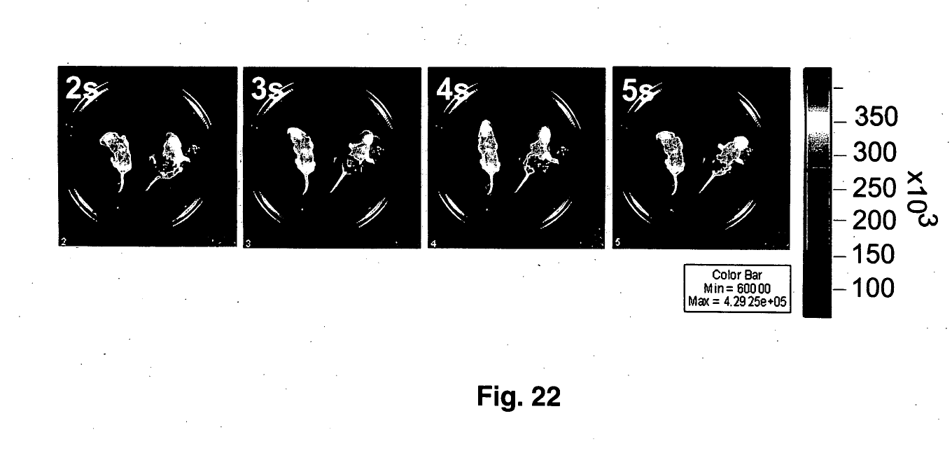

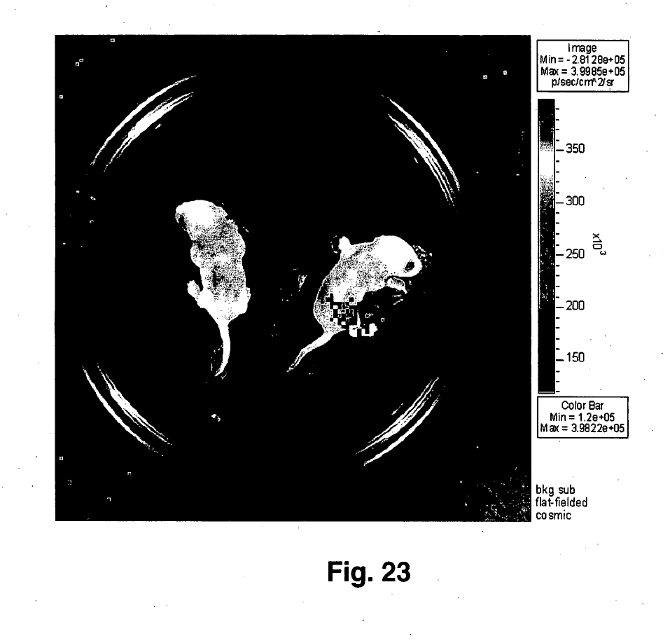

Bioluminescence was also detected in transgenic mice expressing mitochondrially targeted GFP-aequorin. To image Ca²⁺-induced bioluminescence from within a transgenic mouse expressing GFP-aequorin reporters, coelenterazine needs to be injected (e.g. intra-peritoneally or by the tail vail). We tested whether local Ca²⁺ dynamics can be detected in live mice, after injecting neonates intra-peritoneally with coelenterazine and then imaging them at different time resolutions. Transgenic mice were directly compared to non-transgenic mice, by imaging a series of consecutive images consisting of 5-second acquisition frames. Grayscale photographs of the mice were first collected to follow mouse movements and to correlate these images with the overlay of bioluminescence images. We found that sequence files showed dynamic emission of bioluminescence correlating to mouse movements (FIGS. 20 and 21). The bioluminescence detected was characteristic of light having short flash kinetics as it appeared in single frames and corresponding to mouse movements. At higher time resolutions, 2-4 second frames (FIG. 22), Ca²⁺ signals could also be detected inside of the mitochondrial matrix of freely moving mice. We could also detect signals with a good signal-to-noise ratio using a 1-second acquisition time (FIG. 23). Short flashes of bioluminescence were detected in areas such as the hind legs, forelimbs, spinal cord, cerebral trunk and other dorsal areas of the body. In all cases, these Ca²⁺ signals were synchronized with regions of the body where skeletal muscle contraction-relaxation was occurring according to what is seen in the greyscale photographs taken prior to each luminescent image. It has only been shown very recently using two-photon microscopy in vivo analysis of fluorescent Ca²⁺ reporters, that skeletal muscle mitochondria take up and release Ca²⁺ during muscle contraction-relaxation. However, this approach was more invasive, because it utilized excitation light and electroporation techniques as a means to deliver DNA into the muscle fibers and because it was necessary to detach the distal tendon and surgically expose the muscle fibers of interest for the in vivo imaging using two-photon microscopy. Furthermore, it was necessary to maintain mice under anesthetics during the procedure and there is some evidence suggesting that volatile anesthetics can directly affect complex proteins within the mitochondrial respiratory chain. Nevertheless, these studies produced images of mitochondrial Ca²⁺ uptake and release during contraction-relaxation of the muscle fibers with very high spatial resolution.

Potential Application 1: Detection of Calcium Fluctuations Associated with Morphological or Developmental Changes in Neurons Using Genetically Targeted GFP-Aequorin.

Ca²⁺ is believed to be a central modulator of growth cone motility in neurons. Using photolabile caged Ca²⁺, studies have shown that transient elevations in Ca²⁺ positively modulates growth cone motility. GAP43 is believed to be an important protein involved in axon guidance during development and in regeneration following nerve injury. The axonal/growth cone protein, GAP43 is believed to be associated with calmodulin at the membrane and is activated by the transient Ca²⁺ increase believed to be associated with growth cone motility. Hence, GFP-Aequorin targeted to the post-synaptic density protein, GAP43, would enable localised calcium signalling and corresponding morphological changes associated with neurite outgrowth to be monitored during development and in neural regeneration.

Potential Application 2: Use of Targeted GFP-Aequorin to Monitor Receptor Function, those Permeable to Ca²⁺ or those Associated with a Localised Increase in Calcium Concentration.

Example 1: Using GFP-Aequorin fused to PSD95 for specifically monitoring localised Ca²⁺ increases after NMDA receptor activation and abnormalities associated with calcium signaling in neurological diseases, such as Alzheimer's diseases.

Example 2: Using targeted and low affinity GFP-Aequorin for selective detection of high calcium concentration microdomains in cell population studies for high-throughput screening (i.e. In multi-well format, 96, 386 or 1544 well plates) of pharmacological agents or chemical compound, or combinatorial compound libraries for detection of pharmacological candidates that could treat neurological diseases. This technique is considerably more powerful than those utilising fluorescent dyes that sense calcium.

Potential Application 3: Preclinical Trial Studies:

Dynamic images of Ca²⁺ activity can be acquired by in vivo whole animal bioluminescence imaging of living subjects (FIGS. 15-18). The light produced penetrates mammalian tissues and can be externally detected and quantified using sensitive light-imaging systems. GFP-aequorin signals emerging from within living animals produce high signal-to-noise images of Ca²⁺ fluxes that can be followed with high temporal resolution. This technique represents a powerful tool for performing non-invasive functional assays in living subjects and should provide more predictive animal data for preclinical trial studies.

### DISCUSSION

This invention thus provides a modified bioluminescent system comprising a fluorescent molecule covalently linked with a photoprotein, wherein the link between the two proteins has the function to stabilize the modified bioluminescent system and allow the transfer of the energy by Chemiluminescence Resonance Energy Transfer (CRET). In a preferred embodiment, the bioluminescent system comprises a GFP protein covalently linked to a aequorin protein, wherein the link between the two proteins has the function to stabilize the modified bioluminescent system and to allow the transfer of the energy by Chemiluminescence Resonance Energy Transfer (CRET).

This invention provides a composition comprising a recombinant polypeptide, wherein the composition has the functional characteristics of binding calcium ions and permitting measurable energy, said energy depending of the quantity of calcium bound and of the quantity of polypeptides in said composition in absence of any light excitation.

This invention incorporates a peptide linker having the function after translation to approach a donor site to an acceptor site in optimal conditions to permit a direct transfer of energy by chemiluminescence in a polypeptide according to the invention. Preferred linkers are described in PCT Application WO01/92300, published 6 Dec. 2001, U.S. application Ser. Nos. 09/863,901 and 10/307,389 the entire disclosures of which are relied upon and incorporated by reference herein.

Thus, this invention utilizes a recombinant polypeptide of the formula:

GFP-LINKER-AEQ;

wherein GFP is green fluorescent protein; AEQ is aequorin; and LINKER is a polypeptide of 4-63 amino acids, preferably 14-50 amino acids.

The LINKER can comprise the following amino acids:

(Gly Gly Ser Gly Ser Gly Gly Gln Ser)ₙ, wherein n is 1-5. Preferably, n is 1 or n is 5. LINKER can also include the amino acid sequence Ser Gly Leu Arg Ser.

Another recombinant polypeptide for energy transfer from aequorin to green fluorescent protein by Chemiluminescence Resonance Energy Transfer (CRET) following activation of the aequorin in the presence of Ca⁺⁺ has the formula:

GFP-LINKER-AEQ;

wherein GFP is green fluorescent protein; AEQ is aequorin; and LINKER comprises the following amino acids:

(Gly Gly Ser Gly Ser Gly Gly Gln Ser)ₙ, wherein n is 1-5; and wherein the fusion protein has an affinity for Ca²⁺ ions and a half-life of at least 24 hours. The LINKER can include the amino acid sequence Ser Gly Leu Arg Ser. In addition, the recombinant polypeptide can further comprise a peptide signal sequence for targeting the recombinant polypeptide to a cell or to a subcellular compartment.

This invention also provides polynucleotides encoding recombinant polypeptides as described above.

Plasmids containing polynucleotides of the invention have been deposited at the Collection Nationale de Cultures de Microorganismes (“C.N.C.M.”), Institut Pasteur, 28, rue du Docteur Roux, 75724 Paris Cedex 15, France, as follows:

| Plasmid | Accession No. | Deposit Date |
|---|---|---|
| VA | I-3665 | Aug. 24, 2006 |
| RA | I-3666 | Aug. 24, 2006 |
| PSDGA | I-3159 | Feb. 12, 2004 |

VA or Venus-aequorin (CNCM I-3665) is a plasmid containing CMV promoter and venus-aequorin in an over expression plasmid that allows the hybrid protein VA to be produced in eucaryotic cells.

RA or RFP-aequorin (CNCM I-3666) is a plasmid containing a CMV promoter and mRFP1-aequorin. It is an over expression plasmid that allows the hybrid protein RA to be produced in eucaryotic cells.

E. coli cells comprising the PSDGA plasmid can be cultivated in LB medium at 37° C., in conventional cell culture conditions.

Ca²⁺ transients participate in a diverse array of signaling pathways, which are necessary for development, neuronal plasticity, neurotransmission, excitotoxicity and other important processes. Encoding Ca²⁺-dependent activity at the cellular and subcellular level is complex, involving spatial, temporal and quantitative factors. Here we demonstrate an approach to quantitate local Ca²⁺ signaling by visualizing bioluminescence of the genetically encoded recombinant polypeptide, GFP-aequorin. By fusion to a signal peptide or to proteins important in synaptic transmission, a set of Ca²⁺ sensitive recombinant polypeptide that can be used to directly visualize local Ca²⁺ signaling in single cells or in more complex systems have been constructed. By detection of bioluminescence, it is possible to measure local Ca²⁺ signals having different spatial-temporal properties, with a good signal-to-noise ratio.

Toxicity and Degradation of the Recombinant GFP-Aequorin Polypeptide.

This invention shows that this bifunctional recombinant polypeptide can enable the investigation of calcium activities in neuronal networks, in specific subcellular compartments and in cellular microdomains, of dissociated cell cultures, acute or organotypic slices and transgenic animals.

The expression of this recombinant polypeptide of the invention in different biological systems has shown that there is no toxicity in vitro or in vivo, at the one-cell, the tissue or even the whole transgenic animal or plant stage. This result is also achieved when said recombinant polypeptide is strongly expressed. Therefore, no toxicity has been reported when the recombinant polypeptide is expressed from early stages of development right trough to the adult, in transgenic mice. Moreover, despite the fact that the recombinant polypeptide is neither an endogenous protein nor does it contains endogenous components, the expression does not perturb normal physiological function. No behavioural defects have been reported. The low Ca²⁺ binding affinity of GFP-aequorin (see FIG. 2C), does not cause significant perturbation to Ca²⁺ signals.

Another feature of this recombinant polypeptide is that despite its exogenous origin, there is no degradation of said recombinant protein according to the cell type where it is expressed or the developmental stage.

The characteristics of these bifunctional recombinant polypeptides make them a useful tool to study localised Ca²⁺ signaling in disease processes. In particular, a targeted bifunctional recombinant polypeptide could be used to monitor the function of a receptor when associated to a localised increase in the concentration of Ca²⁺, such as studies of NMDA receptor function in neurodegenerative disease models, using the PSDGAmut protein, which targets to the postsynaptic density, including to NMDA receptors.

The characteristics of these bifunctional recombinant polypeptides make them an extremely useful tool for the visualisation of dynamic or fluctuating changes in Ca²⁺ at central synapses that may occur over prolonged periods, such as between neuronal connections during development or with altered network properties that accompany learning and memory, aging and changes associated with chronic exposure to drugs.

The characteristics of these bifunctional recombinant polypeptides make them an extremely useful tool in diagnostics or in the drug discovery process. In particular, a targeted bifunctional recombinant polypeptide could be used to monitor specifically the function of a receptor that is associated with a known localised increase in the concentration of Ca²⁺. GFP-Aequorin could be targeted to a specific cellular site associated with a high Ca²⁺ concentration microdomain and used to monitor drug effects in a more specific fashion in cell populations using high-throughput screening. Current methods utilise non-targeted fluorescent indicators for this purpose and are subject to problems associated with photobleaching, phototoxicity, low signal-noise ratio, dye leakage, heterogenous dye distribution, detection of other calcium dynamics indirectly associated with receptor activation, eg. Calcium induced calcium release. A recent study of high-throughput drug candidate screening, demonstrated that the non-targeted version of GFP-Aequorin has a significantly better signal-to-noise ratio than that offered by fluorescent probes. This is particularly advantageous for monitoring small Ca²⁺ fluxes such as those induced by activation of inhibitory G-proteins, which are implicated in a number of neurological diseases (Niedernberg et al. 2003). GFP-Aequorin could therefore also be useful for monitoring Ca²⁺ fluxes associated with the nicotinic receptor subtype, known as the alpha-7 containing nicotinic receptor, which are difficult to detect with fluorescent indicators.

Optical detection of Ca²⁺ can offer a simple approach for visualizing ‘real-time’ dynamic activity and long-term cellular changes that are associated with specific phenotypes or pathologies. For example, characterizing the spatiotemporal specificity of Ca²⁺ profiles in synaptic function is important to understand the mechanisms contributing to perturbed neuronal Ca²⁺ homeostasis, which has been implicated in schizophrenia and early events associated with the onset of neurodegenerative diseases such as Alzheimer's, Parkinson's and Huntington's diseases (Lidow, 2003; Mattson and Chan, 2003; Stutzmann et al., 2004; Tang et al., 2003).

An advantage of working with bioluminescence is that recordings with very high time resolution can be undertaken over extended periods. For long-term recordings, the consumption of the aequorin photoprotein needs to be taken into consideration. However we have been able to undertake long-term recordings (for up to 9 hours) with very high time resolution (1 ms resolution) on tissue slices, including cortical slices. Overall, we find that photoprotein consumption and the level of sensitivity for detecting variations in Ca²⁺ is relevant to the amount of recombinant polypeptide expressed, to the localization of the expressed recombinant polypeptide and to the type of coelenterazine analogues used (Shimomura, 1997; Shimomura et al, 1993). This can vary from application to application, from cell to cell and needs to be optimized in each case, much the same as it needs to be for fluorescent probes. Ca²⁺ sensitive bioluminescent recombinant polypeptides could also represent the reporter of choice in studies on biological systems that are sensitive to light.

Comparison to Ca²⁺ Sensitive Fluorescence Reporter Systems

Visualization of fluorescence requires an external light source for light excitation of the fluorescent molecule (light is generated through absorption of radiation), which causes photobleaching, phototoxicity and auto-fluorescence (high background with variable intensity over time). Bioluminescent reporters have significantly greater signal-to-noise ratio than fluorescent reporters. A high signal-to-noise ratio is desirable for better quality data in imaging applications, as low signal levels are less affected by interference.

Detection of bioluminescence is a non-invasive way to monitor biological processes in the living intact animal. Fluorescent reporters are a problem because light excitation on tissues results in a large degree of autofluorescence. Furthermore, light must pass through tissue to excite fluorescent molecules and then light emitted of a longer wavelength must pass back through tissue to be seen by the detector. Genetically encoded Ca²⁺ sensitive fluorescent reporters are sensitive to temperature and pH or contain calmodulin, which is a native protein of mammalian cells. Over-expression of calmodulin could produce a phenotype in transgenic animals.

Comparison of the Aequorin-Coelenterazine System to the Luciferase-Luciferin System for Whole Animal In-Vivo Bioluminescence Imaging

The luciferase reporter system has been utilized for following infection processes, tumour progression and gene expression in the living animal. This system is now well established as an animal model for testing drug candidates in clinical trials. Our recent data in single-cells and brain slices, suggests that using GFP-aequorin as a bioluminescent reporter at the single-cell level compares favourably to the luciferase system. As GFP-aequorin is a recombinant polypeptide that can be used as reporter of Ca²⁺ activities, we utilize recombinant polypeptide expressing-mice for following more dynamic changes in the living mouse and show that it is possible to detect GA bioluminescence with good temporal resolution at the whole animal level. Elevation of [Ca²⁺]ᵢ concentrations in specific cellular domains, are correlated with the onset of many pathological processes. Calcium is also an important signalling ion involved in development and apoptotic processes. We have found in our studies that large Ca²⁺ changes are associated with apoptotic events. An early event in this process is believed to involve mitochondrial release of Ca²⁺. Since Ca²⁺ levels in the mitochondria of a healthy cell at rest are generally close to cytosolic [Ca²⁺], an increase in mitochondrial Ca²⁺ levels are likely to mark very early changes that lead to cell death. Mitochondria are now regarded unequivocally as the cells biosensor and changes in mitochondrially Ca²⁺ handling are central to this property. This will offer an alternative approach to the luciferase reporter and broaden the applications possible with this technology. Combined with the continual improvement in detector technology and with eventual improvements in the chemistry of the co-factor, these recombinant polypeptide expressing-mice have the potential to become a powerful system of analysis in all aspects of biology and for clinical testing of new treatment modalities.

Finally, we have constructed a transgene encoding mtGA that is under the control of the strong promoter, β-Actin and also the Lox-Stop-Lox system. Results shown here indicate that the CRE-regulated transgene is functional in the mouse embryo. By inducing cell-type specific expression of the reporter protein at any developmental stage in the mouse, we can now study more precisely cellular processes occurring inside the living animal. For example, we can utilise a recombinant virus containing CRE that is under the regulation of a cell specific promoter. Oncogenic processes or tumour progression could therefore be studied in a selected cell type or tissue in acute or organotypic slices or at the whole animal level.

A major advantage is that these animals could be crossed with mutant animals or models of disease to investigate different pathologies. These animals can also provide a specific source of labelled tissues, cells and tumours for ex-vivo or in-vitro studies

The genetically encoded, Ca²⁺-sensitive bioluminescent hybrid protein, GFP-aequorin, is based on the light emitting system that evolved in the jellyfish [17]. In contrast to aequorin alone, GA can be localized by the fluorescence of GFP, has higher stability, produces higher levels of Ca²⁺-induced bioluminescence and has peak emission at green wavelengths.

This invention accomplishes a major objective by showing for the first time that GFP-aequorin can be used to monitor, non-invasively, Ca²⁺-signaling in real-time within the intact animal during motion. While GA gives a high degree of sensitivity for non-invasive detection of mitochondrial Ca²⁺-signaling events with high temporal resolution in freely moving transgenic animals, there is limited sensitivity to the detection of GA in deep tissues, like the heart and brain, because light in the blue/green spectrum is strongly absorbed by tissues, such as hemoglobin and bone [10, 18, 19]. As a consequence, the bioluminescence of GA when expressed in transgenic animals is mostly detected from superficial tissue sites and there is a general lack of sensitivity in deeper tissues, like the heart and brain.

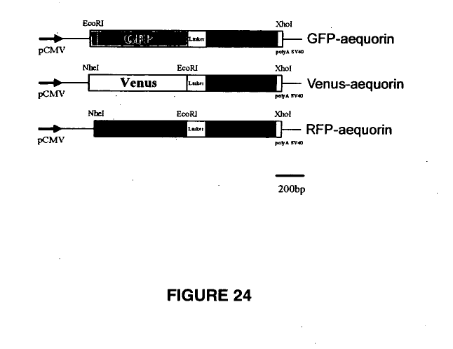

Accordingly, red-shifted bioluminescent Ca²⁺ reporters were constructed by fusing aequorin with the yellow fluorescent protein, Venus (VA) [21] and the monomeric red fluorescent protein, mRFP1 (RA) [22]. That is, it was decided to introduce YFP (Yellow Fluorescent Protein) and mRFP1 (monomeric Red Fluorescent Protein 1) in place of GFP for the development of constructs with different spectral properties (FIG. 24). The spectral properties of these new hybrid proteins were characterized, and it was found that the luminescence energy resulting from aequorin catalysed oxidation of coelenterazine is transferred to each fluorophore with very different levels of efficiency. After evaluating the attenuation of light emission by these probes through different tissues, it was discovered that these reporters can improve the detection of Ca²⁺ activities in deeper tissues, like the heart and brain.

More particularly, real-time visualization of calcium (Ca²⁺) dynamics in the whole animal now enables important advances in understanding the complexities of cellular function. It has been discovered that transfer of aequorin chemiluminescence energy to Venus (VA) is highly efficient and produces a 58 nm red shift in the peak emission spectrum of aequorin. This substantially improves photon transmission through tissue, like the skin and thoracic cage. While, the Ca²⁺-induced bioluminescence spectrum of mRFP1-aequorin (RA) is similar to aequorin, there is also a small peak above 600 nm corresponding to the peak emission of mRFP1. Small amounts of energy transfer between aequorin and mRFP1 yields an emission spectrum having the highest percentage of total light above 600 nm, compared to GA and VA. Accordingly, RA is also detected with higher sensitivity from brain areas. VA and RA, therefore, improve optical access to Ca²⁺-signaling events in deeper tissues, like the heart and brain, and offer insight for engineering new hybrid molecules.

This invention shows that, like for GFP-aequorin, the Ca²⁺-induced chemiluminescence of Venus-aequorin is the result of a highly efficient intramolecular energy transfer from the donor moiety of aequorin to the fluorescent acceptor, Venus. In contrast, RA emits most of its light in the blue spectrum, which probably comes from aequorin light emission, whereas only a small degree of non-radiative or radiative energy transfer is likely to contribute to the far red emission. Moreover, mRFP1-aequorin was the only bioluminescent reporter able to emit light in the far red spectrum (≧650 nm). The low efficiency of energy transfer detected between aequorin and mRFP1 is probably related to the small spectral overlap between the emission spectra of aequorin and the excitation spectra of mRFP1. In addition, the structural conformation and dynamics of the mRFP1-aequorin molecule may shorten the interchromophore distance for energy transfer and/or be modulated by internal vibrational motion that allows CRET in small amounts to be produced in the red and far red range [32, 33].

This invention further shows that the luminescence emitted from the three hybrid proteins was able to be efficiently detected when emitted from subcutaneous regions. Corresponding to what is known for the spectral properties of tissues, GA emission is highly attenuated from subthoracic regions, whereas Venus-aequorin and mRFP1-aequorin had spectral properties that significantly improved the transmission of Ca²⁺-induced light emission through animal tissues, particularly from deeper regions, like underneath the thoracic cage. Given the spectral characteristics of Venus-aequorin, this reporter is suitable for use in applications requiring the detection of light from subcutaneous areas or from the thoracic cavity. The highly efficient energy transfer and spectral properties of Venus-aequorin indicates that this reporter is appropriate for studies on liver function, for example.

In contrast, Venus-aequorin light emission is highly attenuated when passing through the skull, whereas the transmission of mRFP1-aequorin is more efficient. Other studies have reported that photons transmitted through hard bone tissue are far red-shifted and it was found in connection with this invention that most of the light detected from mRFP1-aequorin when placed underneath the skull is from the far red spectrum (≧610 nm) [19, 34]. It is possible to detect Ca²⁺ concentration increases in the brain of young transgenic mice (<10 days postnatal) expressing GFP-aequorin when the skull is still relatively thin. However, activity in the brain was difficult with this probe. Non-invasive procedures, such as imaging of the intact brain of mice in living animals, including adults, can be more easily investigated with transgenic mice expressing mRFP1-aequorin.

Other studies have produced red-shifted versions of the luciferase reporters (i.e. Renilla and firefly) and evaluated their transmission properties through animal tissue by the injection of transfected cells expressing these bioluminescent probes in the mouse liver and lungs [11, 19]. These studies are feasible, because there is always a sufficient level of O₂ or ATP present in cells to facilitate light emission. Similar experimental paradigms with the aequorin system would be difficult because the light reaction only occurs when there is a transient increase in the Ca²⁺ concentration over and above basal levels of Ca²⁺ (>300 nM). More physiological studies require the generation of transgenic mice expressing such reporters, characterized in this invention.

Fusions between aequorin and some of the newly reported red fluorescent proteins, such as mStrawberry and mCherry molecules, can also be employed in this invention. Better results than mRFP1-aequorin may be expected because of the larger overlap between aequorin emission/fluorescent excitation as well as their higher quantum yields and extinction coefficients [35, 36].

The efficiency of light emission can also vary according to the type of coelenterazine analog used. This invention is exemplified using the native version of coelenterazine. However, other coelenterazine analogs reconstituted in combination with mRFP1-aequorin may improve CRET, such as v-coelenterazine, which has an emission maximum at 512 nm [37]. In this case, other properties may need to be considered, such as stability and pharmacokinetic studies described for different substrates in whole animals [38].

Another alternative is a three-way fusion protein, comprising of mRFP1-Venus and aequorin, for maximum CRET efficiency, in order to improve the efficiency of energy transfer and amount of light produced in the far red spectrum [39]. An additional application with these different coloured reporters is to simultaneously monitor Ca²⁺ dynamics in different subcellular compartments or cell types.

The Venus-aequorin and mRFP1-aequorin reporters make it possible to undertake non-invasive whole animal imaging of heart and brain activity, respectively. Venus-aequorin is a more appropriate probe for studies of Ca²⁺ activity in tissues such as the skin, muscles, or heart. Alternatively, mRFP1-aequorin is the preferred probe for imaging of cerebral activity, which will open the field of optical imaging during a learning paradigm. Since light emission in the blue/green spectrum has a reduced capacity for tissue penetration, GFP-aequorin is not well adapted for whole animal deep tissue imaging. These hybrid bioluminescent reporters can be used as genetically targeted Ca²⁺ sensors to specific cellular and/or subcellular domains to simultaneously study Ca²⁺ dynamics in a non-invasive manner from different domains in medical, pharmaceutical, and environmental applications.

The hybrid bioluminescent reporters will now be described in greater detail in the following additional Examples.

### EXAMPLE 6

### Hybrid Gene Constructions

As described previously, aequorin has been codon optimised for expression in mammalian cells [17]. Venus-aequorin (VA) was constructed by creating a fusion between the GFP variant, yellow fluorescent protein known as Venus, which was kindly provided by Dr. A. Miyawaki [21], and the aequorin gene. Venus and aequorin are separated by a flexible linker (45 amino acids as described for G5A previously) [17]. The gene sequence of Venus was amplified by PCR using oligonucleotides that allowed (i) insertion of a site for NheI before the start of the initiation codon and (ii) removal of the stop codon and insertion of a site for EcoRI. The nucleotide sequence of the forward primer was 5′ACACTATAGMTGCTAGCTACTTGTT3′, which contains a NheI site and the reverse primer was 5′AGAGGCCTTGMTTCGGACTTGTA3′, which contains a site for EcoRI. The PCR fragment containing the gene of Venus was then inserted NheI/EcoRI in place of PSD95, in pPSDGA [23] to give the plasmid VGA.

The flexible linker (at the end of GFP) plus aequorin were together amplified by PCR from the G5A plasmid [17] using the forward primer 5′GACGAGCTGTACGAATTCGGCG3′, which included a site for EcoRI, and the reverse primer 5′CTGGMCAACACTCMCCCTATCT3′. The resulting PCR fragment linker-aequonin was then digested EcoRI/XhoI and inserted in place of GA in the plasmid VGA, to obtain pVA. mRFP1-aequorin (RA) was constructed by creating a fusion between the monomer RFP1 gene from the reef coral Discosoma sp. (pRSET1 DNA vector was kindly provided by Dr. R Tsien) [22] and the aequorin gene. Similarly to VA, mRFP1 and aequorin are separated by a flexible linker. A phosphorylated linker with the oligonucleotides: 5′pCGCGCCGAGGGCCGCCACTCCACCGGCGCCAAAGAATTCACGCGTG and 5′pCGCGCACGCGTGAATTCTTTGGCGCCGGTGGAGTGGCGG CCCTCGG3′ was introduced at the BssHII single site in RSET1 vector and mRFP1 was then removed by NheI/EcoRI digestion and inserted in place of the Venus sequence in the NheI/EcoRI sites of the VA plasmid. Both reporter genes were placed under the control of the ubiquitous cytomegalovirus promoter (CMV) and all sequences were verified by DNA sequencing.

### EXAMPLE 7

### Preparation of Cell Lysates Containing the Hybrid Protein

COS7 (Kidney cells, monkey) and neuroblastoma (Neuro2A, mouse) cells were grown in DMEM supplemented with 10% (vol/vol) heat-treated FCS, 2 mM glutamine and 50 units/mL of penicillin/streptomycin (Invitrogen, Life Technologies) at 37° C., in a humidified atmosphere containing 5% CO₂. Cells were transiently transfected for 24 to 48 hrs using the FuGENE 6 transfection reagent (Roche, Diagnostics) at a DNA/FuGENE ratio of 1:2. Following transfection, cells were washed (×3) with PBS and harvested using a cell scraper. The cell suspension was transferred into a reconstitution buffer containing 10 mM mercaptoethanol, 5 mM EGTA, and 5 μM wildtype (wt) coelenterazine (Interchim, France) in PBS, at 4° C. for 2 hrs in the dark. After that, cells were washed and resuspended in a hypo-osmotic buffer containing 20 mM Tris/HCl, 10 mM EGTA and 5 mM mercaptoethanol in dH₂O and a protease inhibitor, EDTA-free (Roche, Diagnostics). Cell membranes were lysed by three freeze-thaw cycles, followed by passage through a 26 ga needle. After centrifugation at 13000 g for 1 min, supernatant containing the activated bioluminescent proteins was collected and stored as aliquots at −20° C. until use.

### EXAMPLE 8

### Spectroscopic Characterization of Fusion Proteins Fluorescence and Luminescence

Fluorescence excitation and emission measurements were made using a spectrophotometer (Xenius, SAFAS, Monaco) by mixing cell extracts containing one of the following proteins, GA, VA, and RA, in the presence of 5 mM EDTA with 10 mM CaCl₂ into a standard cuvette holder of the “Safas” spectrofluorimeter. Fluorescence spectra were normalized and all computations and graphs were made using Excel Microsoft® software, except when stated otherwise.

Chemiluminescent measurements were performed using the “Shamrock 163i Spectrograph” combined with a photon counting CCD camera (ANDOR Technology) placed in a dark box. This apparatus is based on a Czerny-Turner optical layout and features a 163 mm focal length, an entrance aperture ratio of f/3.6, and a wavelength resolution of 0.17 nm. Before capture of signals, light passed through a monochromator, allowing the spectral analysis of emitted photons. The acquisition began 5 seconds before injection of 100 mM CaCl₂ solution and continued for 30 s after injection. The spectra signals observed were analysed between 300 and 700 nm by using iStar software (ANDOR Technology). From the CRET curves, total photons emitted at wavelengths ≧470 nm, ≧590 nm, ≧600 nm and ≧650 nm, were counted for each bioluminescent protein. The important feature of this instrument is that the whole spectrum is acquired simultaneously (without scanning) thus ensuring that there is no change during acquisition.

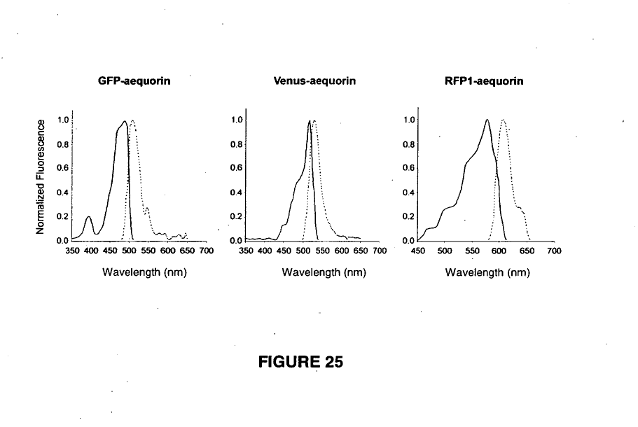

Analysis by spectrofluorometry of the hybrid proteins indicates that the excitation and emission spectra of GA, VA, and RA (FIG. 25A-C) correspond to GFP, Venus and mRFP1, respectively [21, 22]. The excitation/emission maxima for each of the hybrid proteins was 488/509 nm (FIG. 25A), 516/528 nm (FIG. 25B) and 578/607 nm (FIG. 25C), for GA, VA, and RA, respectively. Thus, the spectral properties of each fluorescent protein are not modified when they are expressed as N-terminal fusion proteins to aequorin [21, 22]. As expected for a non-targeted reporter, expression of the hybrid proteins, GA, VA, and RA in COS7 cells, Neuro2A cells, and hippocampal neurons was found to be relatively diffuse in the cell cytoplasm and nucleus, without evidence of protein aggregation or cellular toxicity.

### EXAMPLE 9

### Characterization of the Fusion Proteins

### Kinetic Response and Ca²⁺ Binding Affinity

Aliquots of the cell lysate containing the bioluminescent protein were dispensed into wells containing different free Ca²⁺ concentrations (between 37.5 to 13500 nM) and the variation of light emission (RLU) was plotted as a function of time during 20 seconds.

In addition, one hundred μL of EGTA buffered solutions (Molecular Probes, Inc.) having varying concentrations of free Ca²⁺ as calculated by the WEBMAXC program (see stanford.edu/˜cpatton/webmaxcE.htm) [24], were placed in the wells of white opaque 96-well plates. Background luminescence activity was measured for 5 seconds and then a 10 μL aliquot of cell lysate (containing the active photoprotein), was injected into wells and the light intensity was continuously recorded for 30 s (L, rate of light emission, counts/s). At this point, a saturating CaCl₂ solution (100 mM) was injected and recording of the light intensity was continued until all the photoprotein had been consumed and light levels had returned to baseline (Lₘₐₓ). Light emission obtained for each reporter (VA, RA, and GA) was then expressed as the fractional rate of photoprotein consumption (L/Lₘₐₓ) as described previously [27]. For concentrations below 1100 nM, the value for L was taken at 5 s after injection, at the point where the mixed sample is equilibrated. For samples above 1100 nM, L was taken at the peak of the light intensity. In all cases, Lₘₐₓ was calculated as the total amount of aequorin light that can be emitted by the sample after discharging all of the available aequorin from and including the time point where L was taken.

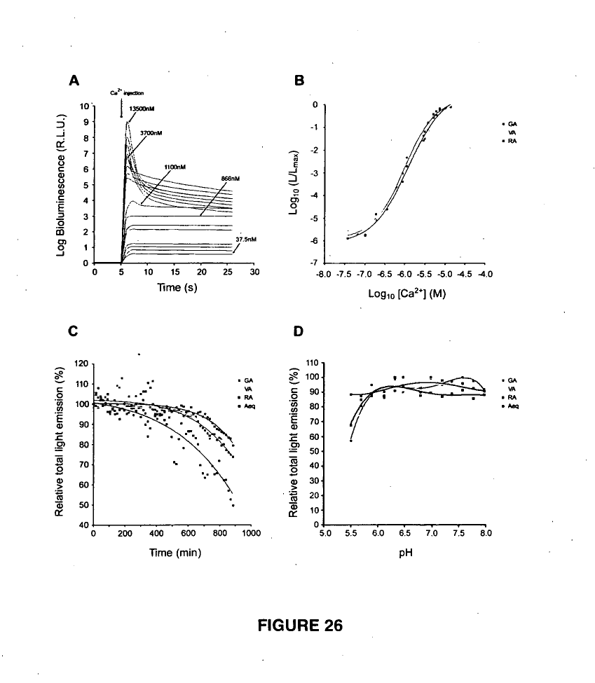

When aequorin (apo-aequorin+coelenterazine) binds Ca²⁺, it becomes ‘consumed’ in the course of its light emitting reaction and decomposes into apoaequorin, coelenteramide and CO₂. Apoaequorin can be converted into its original aequorin in the absence of Ca²⁺ providing there is available coelenterazine and O₂, but the reaction is very slow. The rate of the bioluminescence reaction is a function of the calcium concentration [25, 26]. In saturating conditions, the rate constant for aequorin consumption is 1 sec⁻¹. Accordingly, the rate of the light reaction (and its consumption) of each photoprotein increases with the [Ca²⁺] (37.5 to 13500 nM) (FIG. 26A). However, at [Ca²⁺] from 37.5 nM to 866 nM, the reaction rate is extremely slow and light emission remains relatively constant.

Solutions of activated photoprotein were, therefore, prepared with a free [Ca²⁺] of 866 nM, where the intensity of the light emission is highest, in order to assess the level of light transmission through different filters and animal tissues, without decay of the light intensity. The Ca²⁺-binding affinities of VA and RA were also determined (FIG. 26B) and are similar to GA [23] and those reported for aequorin (Kd=10 μM) [26-28]. Hence, fusing aequorin to different fluorescent proteins, such as GFP, Venus and mRFP1, does not interfere with the Ca²⁺ induced intra-molecular reaction of aequorin, which oxidises the bound coelenterazine.

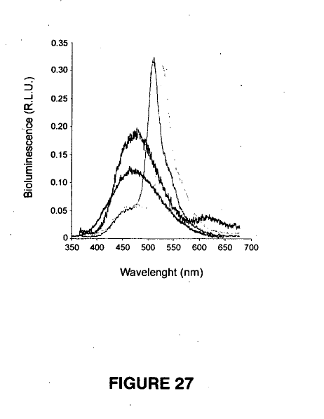

In the presence of Ca²⁺ ions and coelenterazine, a non-radiative energy transfer between the excited oxyluciferin and a chromophore will depend on the donor lifetime, the distance between donor and acceptor dipoles, the relative orientation of dipoles and the degree of emission/excitation spectral overlap [20]. Emission spectra of the different chimeras were analyzed using a highly sensitive spectroscopic detector, which provides high wavelength accuracy, with a resolution of 0.17 nm (FIG. 27). The new fusion proteins were compared to aequorin and GA (formerly G5A) [17]. The emission spectrum of aequorin shows a broad peak with a A max of 469 nm. At 50% of that maximum light emission of aequorin, the peak has a bandwidth of approximately 115 nm. In contrast, the peak emission wavelength of GA occurs in the green, which corresponds to the fluorescence emission for GFP (λₘₐₓ=509 nm), with a small shoulder corresponding to the peak emission of aequorin. At 50% of the maximum light emission of GA, the peak has a narrow bandwidth of approximately 40 nm, similar to previously reported values [17].

The bioluminescent spectra for VA has an emission maximum that is shifted to yellow, indicating that the Ca²⁺-induced chemiluminescent reaction produces light emission coming from the ‘fluorescent’ protein, Venus (A=528 nm). Similarly to GA, at 50% of the maximum light emission, the peak has a narrow bandwidth of 43 nm. In contrast, the spectrum of the Ca²⁺-induced bioluminescence of RA largely corresponded to that of aequorin, with a slightly narrower bandwidth (95 nm). However, the luminescence emission curve of RA appeared asymmetrical and higher in intensity compared to aequorin with a small peak in the red corresponding to the emission maximum of mRFP1 (λₘₐₓ=607 nm). According to the CRET spectra, RA emits 10% of its light at wavelengths greater than 600 nm, which is two-fold higher in comparison to VA. At higher wavelengths, the difference of emission between RA and VA is even greater, demonstrating that RA emits light in the far red spectrum (≧650 nm). In the case of the RA hybrid, one can speculate that most of the light intensity is emitted directly by aequorin. However, a small amount of energy transfer does occur (<10%) by a radiative/non-radiative process, resulting in emission above 600 nm, corresponding to emission by RFP.

The efficiency of energy transfer between the aequorin donor and acceptor fluorophore was determined for each reporter by calculating the ratio of light intensities at the A max of the acceptor emission (I_{A}) to that of the donor, aequorin moiety (I_{D}) [30]. The results are shown in Table 1.

TABLE 1
Distribution of light emitted by each photoprotein
in broad regions of the light spectrum

| | Relative light detected from CRET activities | | | |
|---|---|---|---|---|
| | for different regions of the spectrum (%) | | | |
| Photoprotein | ≧470 nm | ≧590 nm | ≧600 nm | ≧650 nm |
| GFP-aequorin | 87 | 4 | 2.3 | 0.3 |
| (n = 4) | | | | |
| Venus-aequorin | 85 | 10 | 6 | 0.9 |
| (n = 4) | | | | |
| mRFP1-aequorin | 66 | 13.5 | 10 | 3 |
| (n = 4) | | | | |
| Aequorin | 55 | 2.6 | 1.5 | NA |
| (n = 4) | | | | |

NA = Not assayed

The calculated ratio for GA was found to be 5.5 as previously reported [17, 30]. The ratios calculated for VA and RA were 5 and 0.25, respectively. The high CRET efficiency observed for GA and VA may be relevant to the high degree of spectral overlap between aequorin light emission and the fluorescence excitation curves for the two fluorophores (FIG. 25). However, it may also be important that aequorin and GFP are from the same organism, and Venus is a mutant form of GFP. In contrast, mRFP1 is derived from the Discosoma coral, which may be important if a structural mechanism plays a role in facilitating optimal energy transfer. Energy transfer also involves dynamic parameters and overall stability of hybrid protein.

### EXAMPLE 10

### Stability and pH Sensitivity

To assay the stability of the different probes over time, 10 μL of cell lysate containing one of the different probes, were placed into 60 wells of a white opaque 96-well plate. Then the light emission from the different wells was triggered every 15 min by the addition of 100 μl of 100 mM CaCl₂. Luminescence was recorded over 15 hours.

### ph Sensitivity

Recombinant apo-aequorin is reported to be unstable in the cytosol and has a half life of approximately 20 minutes [29]. In line with previous data [17], these results show that cells expressing the fusion proteins have a better Ca²⁺-triggered bioluminescent activity than those expressing aequorin alone (Table 2).

TABLE 2
The Ca²⁺-induced chemiluminescence activities

| Name | RLU × 10⁶/10 units β-gal |
|---|---|
| pG5A | 9.32 ± 2.01 |
| pVA | 7.9 ± 1.5 |
| pRA | 4.4 ± 1.2 |
| pAeq | 0.10 ± 0.05 |

Results Indicate the Mean ±SEM. β-gal, β-galactosidase.

As suggested previously, this may be partly related to an increased stability of apo-aequorin when it is expressed as a fusion protein rather than alone [17, 29] The stability of light emission from GA, VA, RA and aequorin in cellular extracts over time was, therefore, investigated (FIG. 26C). Indeed, after 15 hours of incubation at room temperature, stability was found to be higher for the hybrid proteins, which had reduced luminescence activities of 20-25% compared to 50% for aequorin. These studies were undertaken in the presence of protease inhibitors, which would explain the greater stability of aequorin in these conditions compared to those reported previously [29].

In addition, the pH stability of VA, RA, GA and aequorin was analysed with prepared Good's buffers, including MES (pH 5.5 to 6.8) and MOPS (6.5 to 8.0). Five μL aliquots of cell lysate containing the bioluminescent protein were placed into the wells of white opaque 96-well plates and 245 μL of different pH buffers was added. Ten μL of 260 mM CaCl₂ (10 mM final Ca²⁺ concentration) was then injected into each well and luminescence was recorded during 120 seconds. All experiments were carried out using the luminometer “Multilabel Reader Mithras LB940” (Berthold Technologies, Germany) at room temperature (25° C.). Each experiment was repeated at least 4 times and the results are given as a mean ±S.E.M. The Ca²⁺-induced. bioluminescent reaction of aequorin is not believed to be influenced by pH in the physiological range (Campbell et al, 1979). Similarly, GA, VA and RA are also relatively insensitive to pH in the physiological range (6.5 to 7.5) (FIG. 26D).

### EXAMPLE 11

### Ca²⁺-Induced Bioluminescence in Whole Animals

The spectral properties of light emitted by each hybrid bioluminescent protein were evaluated with different filters in a whole animal imaging system (In vivo IVIS™ Imaging System 100 Series, Xenogen). Ca²⁺-induced light emission of GA, VA, and RA was measured as follows: an EGTA buffered solution containing 866 nM free Ca²⁺ concentration (a [Ca²⁺] ensuring a constant light intensity) as well as the hybrid protein reconstituted with coelenterazine was prepared and aliquoted in equal amounts into five different wells of a 96-well plate. Different filters selecting for varying light emission wavelengths (BP 470-490 nm, BP 500-520 nm, BP 520-540 nm and LP 590 nm) were each placed over a well containing the Ca²⁺-activated photoprotein mixture. For calibration of the total light emission, one of the wells was quantified in the absence of a filter. The light emission detected from each well was integrated for 120 seconds. At the end of acquisition, a large region of interest (R.O.I.) was drawn over the area of light emission and the relative total photon transmission was calculated as follows: % total photon transmission=[total photon flux (with filter)/total photon flux (without filter)×100], where light was quantified as photons/s using the Living Image™ software (Xenogen) as an overlay on Igor image analysis software (Wavemetrics). Each experiment was repeated at least 4 times.

The level of Ca²⁺-induced bioluminescence from each probe, which could be detected through different tissues were evaluated. To evaluate the tissue transmission properties of the light emitted by the 3 probes, solutions containing the bioluminescent proteins and free Ca²⁺ concentration (as described above), were placed at different tissue sites within 10 week old Swiss mice (Charles River) that had been killed by CO₂ inhalation. Equal aliquots of the photoprotein solutions were placed into two 50 μL transparent plastic tubes. One tube was placed into one of the following regions of the body: (i) subcutaneous, underneath the skin on the ventral side of the animal; (ii) subthoracic, underneath the thoracic cage in the area of the heart; (iii) subcranial, directly underneath the skull in the area of the brain, and the second tube was placed directly outside of the animal's body. Total light emission (photons/sec/cm²/sr) was integrated during 300 seconds using the whole animal bioluminescence imaging system from Xenogen (In vivo IVIS™ Imaging System 100 Series with Spectral CCD camera, Xenogen). Light emission detected from negative controls containing cell lysates, but no hybrid protein, was also determined. The light intensity (photons/sec/cm²/sr) of each photoprotein was calibrated in order to compare the three hybrid proteins. These values were further normalised by calculating the ratio of the light emitted from within the intact animal over the total light emitted from the tube external to the body after substraction of the background light (negative controls).

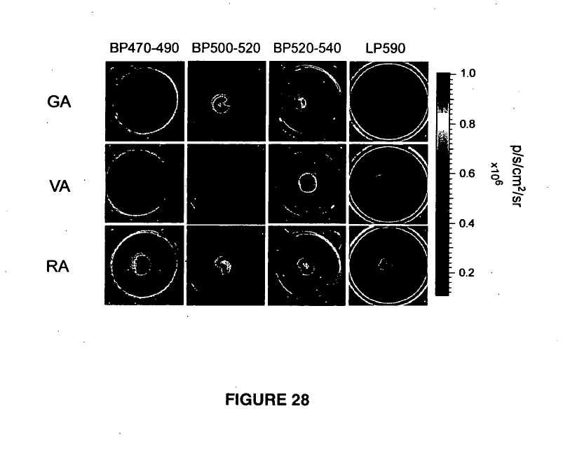

The whole animal bioluminescence imaging system was thus used to determine the transmission of light through different short band-pass (BP) and long-pass (LP) filters. Again, for these experiments, the [Ca²⁺] was maintained at 866 nM because at this concentration the decay of bioluminescent protein activity is minimal and the light signal stays relatively constant over time. The results showed that maximum photon emission for GA, VA, and RA was detected through 500/20 nm, 520/30 nm, and 470/20 nm filters, respectively, confirming the spectroscopy data discussed above. See FIG. 28, and Table 3.

TABLE 3
Band-pass distribution of light emitted by each photoprotein

| | % of Total Photons through the filters | | | |
|---|---|---|---|---|
| | BP470-490 | BP500-520 | BP520-550 | LP590 |
| Photoprotein | nm | nm | nm | nm |
| GFP-aequorin | 10 | 54 | 33 | 3 |
| (n = 4) | | | | |
| Venus-aequorin | 9 | 11 | 70 | 10 |
| (n = 4) | | | | |
| mRFP1- | 48 | 20 | 18 | 14 |
| aequorin | | | | |
| (n = 4) | | | | |

BP = Band pass filter;
LP = Long pass filter.

Importantly, these studies confirmed calculations from the CRET spectra that 14% of RA light emission occurs in the red spectrum (≧590 nm), which suggests that a small degree of energy transfer does take place.

### EXAMPLE 12

### Efficiency of Light Transmission Through Animal Tissues

Previous studies show that transmission of light emitted in the blue/green spectrum (475 to 515 nm) is significantly attenuated in tissue, because it is largely absorbed by components, such as haemoglobin. Alternatively, light above 600 nm provides a higher level of transmission efficiency through mammalian tissues [19]. Therefore, the capacity to detect light emission from GA, VA, and RA through different tissues was evaluated, either underneath (i) the skin (subcutaneous), (ii) the thoracic cage (subthoracic), or (iii) the skull (subcranially). The second tube, which emitted in the range of 2×10⁷ and 1.5×10⁸ photons/second, was then placed next to the animal so that total light output could be quantified and normalised against the levels detected from within the whole animal. This allowed the absorption due to different tissues to be assessed.

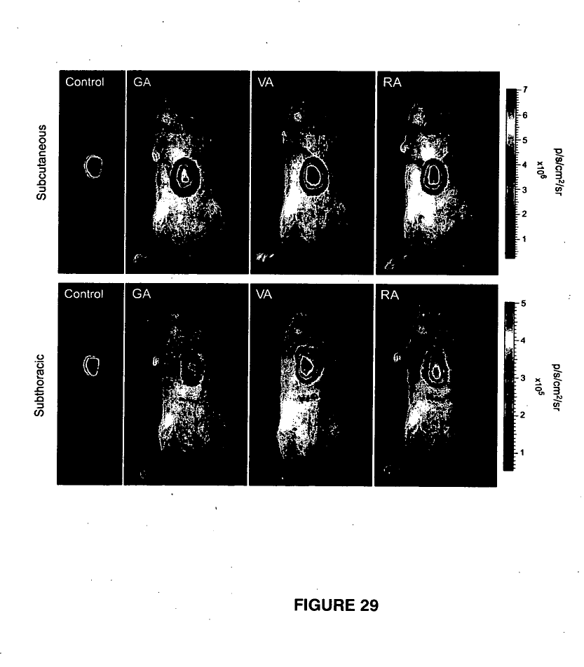

Light emission from the three reporters was readily detected externally with high efficiency when tubes were placed subcutaneously (FIG. 29 and Table 3). However, GA light emission was mostly attenuated by the skin (59.87%), compared to RA (29.47%) and VA (19.71%), as shown in FIG. 29 and Table 4.

TABLE 4
Relative transmission of photoprotein
emitted light across mouse tissues.

| | % of Light transmitted | | |
|---|---|---|---|
| | Subcutaneous | Subthoracic | Subcranial |
| | (Ventral view) | (Ventral view) | (Dorsal view) |
| Photoprotein | (n = 9) | (n = 9) | (n = 12) |
| GFP-aequorin | 40.13 | 1.56 | 0.005 |
| (GA) | (32.5-55) | (0.37-2.26) | (0-0.1) |
| Venus-aequorin | 80.29 | 5.72 | 0.02 |
| (VA) | (61.5-88.9) | (3-9) | (0-0.5) |
| mRFP1-aequorin | 70.53 | 4.86 | 4.6 |
| (RA) | (60.1-79.5) | (2.6-8.5) | (2-6) |

Among the three chimeric proteins, the spectral characteristics of VA light emission, therefore, provided the greatest efficiency to cross tissue over the subcutaneous region. In contrast, light emission from all three reporters was largely attenuated when emitted from deeper tissue, sites like underneath the thoracic cage. From the subthoracic region, a large amount of GA light emission was again attenuated (>98%), while light emission from RA and VA was 3 to 4 fold higher than GA. These studies showed that VA and RA could be detected with relatively similar capacity.

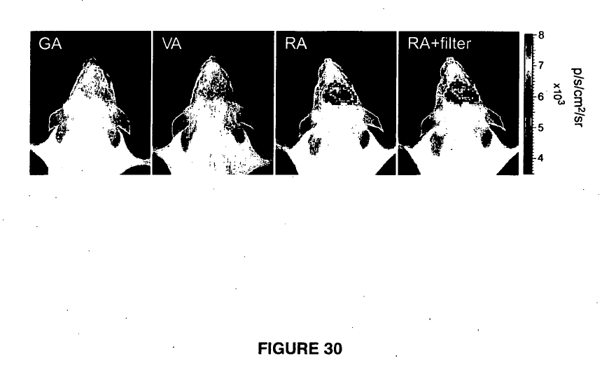

Most of the light emission from GA and VA was attenuated by tissues when tubes containing the activated photoproteins were placed subcranially (FIGS. 30A, 30B and Table 4). While VA and RA had similar capacities to cross tissue from the subthoracic region (5.72 compared to 4.86%), the level of VA light emission was significantly attenuated compared to RA from the subcranial regions (99.98 compared to 95.4%) (FIG. 30B and Table 4). Interestingly, the efficiency for RA light emission to pass through tissues covering the subthoracic and subcranial regions was relatively the same (4.9 compared to 4.6%). Importantly, RA light emission could be readily detected through the mouse skull (FIG. 30C). Furthermore, when the light emission was detected through a filter, it was found to be predominantly from wavelengths greater than 600 nm (FIG. 30D).

These studies confirm the importance of selecting reporters with optimal spectral characteristics and light intensities, relevant to different tissues, when preparing new applications for in vivo imaging.

In summary, Ca²⁺ is a universal second messenger regulating many cell signaling pathways [1, 2]. Optical imaging of Ca²⁺ signaling therefore contributes enormously to our understanding of many biological processes, such as fertilization, neurotransmission, gene expression and muscle contraction. Advances in genomics and proteomics, have been followed closely with a shift towards use of genetically encoded probes for viral mediated transfection or for expression in transgenic animals. Among them are a new class of fluorescent Ca²⁺-sensitive probes, which can be targeted to subcellular regions of the cell, as well as to specific cell types during development and in adult transgenic animals [3-6]. Despite important progress in this field, fluorescent Ca²⁺ sensitive proteins, like the ‘cameleons’[7,8], ‘pericams’ [9] and G-CaMP [6], can only be used in applications that are invasive and restricted to local tissue sites, because they require the input of external radiation for excitation of the fluorophore. Alternatively, a major challenge is to develop whole animal imaging for a real-time analysis of signaling pathways at the molecular level in freely moving animals.

Bioluminescence is light produced from enzyme mediated oxidation of a substrate. Given that mammalian tissues have very low levels of intrinsic bioluminescence, the light from bioluminescent reporters can be detected from within intact organisms with a very high signal-to-noise ratio [10]. Accordingly, whole animal bioluminescence imaging (BLI), using bacterial, firefly or Renilla luciferases, provides a highly sensitive technique for detecting gene expression in small animals [11-13]. Another well known luciferase is the Ca²⁺-sensitive photoprotein, aequorin, which was cloned from the jellyfish, Aequorea Victoria [14, 15]. In contrast to firefly or Renilla luciferases, the light reaction is dependent on Ca²⁺. When Ca²⁺ binds to aequorin, the enzyme undergoes a conformational change that allows oxygen to react with its substrate coelenterazine and this is followed by an inter-molecular chemiluminescence resonance energy transfer (CRET) to GFP, which red-shifts the blue-light of aequorin into the green (λₘₐₓ=509 nm) [16].

### DEFINITIONS

The following terms have the following meanings when used herein:

### Luminescence

Emission of an electromagnetic radiation from an atom or molecule in UV, in visible or IR. This emission results from the transition from an electronically excited state towards a state from weaker energy, generally the ground state.

### Fluorescence

Fluorescence produced by a singlet, very short, excited electronically. This luminescence disappears at the same time as the source from excitation.

### Chemiluminescence

Luminescence resulting from a chemical reaction.

### Bioluminescence

Visible chemiluminescence, produced by living organisms. The invention mimics the system naturally present in the jellyfish, without fixation to a support.

### Bioluminescent System

The bioluminescent system according to the invention is a chimeric tripartite molecule within the middle a peptide linker and a coenzyme (i.e., coelenterazine). The first molecule and the second molecule covalently attached with the linker can be everything if they have for the first a donor site and for the second an acceptor site attached on it (receptors-linker-ligand, antibody-linker antigen). The chimeric protein can be fused to a fragment of tetanus toxin for its retrograde and transynaptic transport on axon by Coen, L., Osta, R., Maury, M., and Brulet, P., Construction of hybrid proteins that migrate retrogradely and transynaptically into the central nervous system. Proc. Natl. Acad. Sci. (USA) 94 (1997) 9400-9405, or fused to a membrane receptor.

### Non-Radiative

No emission of photon from aequorin to the GTP when aequorin is bounded by calcium ions (therefore there is no transmission of blue light by aequorin in the invention, the energy transfer is directly made between the two proteins).

### FRET System

Transfer of energy by resonance by fluorescence (i.e., between two variants of GFP).

### REFERENCES

Fluorescent indicators for Ca²⁺ based on green fluorescent proteins and calmodulin.
Miyawaki, A., Liopis, J., Heim, R., McCaffery, J. M., Adams, J. A., Ikura, M. and Tsien, R. Y. Nature, (1997) Vol. 388 pp. 882-887.

Detection in living cells of Ca²⁺-dependent changes in the fluorescence emission of an indicator composed of two green fluorescent protein variants linked by a calmodulin-binding sequence. A new class of fluorescent indicators.
Romoser, V. A., Hinkle, P. M. and Persechini, A., J. Biol. Chem., (1997) Vol. 272, pp. 13270-13274.

### CRET

Transfer of energy by resonance by chemiluminescence (i.e., fusion protein with GFP-aequorin (jellyfish Aequorea) but without linker or GFP-obeline).

### REFERENCES

Chemiluminescence energy transfer.
Campbell, A. K., in Chemiluminescence: Principles and application in Biology and Medicine, Eds Ellis Horwood, Chichester, UK 1988, pp. 475-534.

### BRET

Transfer of energy by resonance by bioluminescence (i.e., interaction between GFP and luciferase (jellyfish Renilla).

### REFERENCES

A bioluminescence resonance energy transfer (BRET) system: application to interacting circadian clock protein.
Xu, Y., Piston, D. W. and Johnson, C. H. Proc. Natl. Acad. Sci., (USA) (1999) Vol. 96, pp. 151-156.

### BIBLIOGRAPHY

The following references are cited herein. The entire disclosure of each of these references and each of the other references cited herein is relied upon and incorporated herein.
[1]. Berridge M J, Bootman M D, Roderick H L. (2003) Calcium signalling: dynamics, homeostasis and remodelling. Nat Rev Mol Cell Biol 4: 517-529.
[2]. Rizzuto R, Pozzan T. (2006) Microdomains of intracellular Ca2+: molecular determinants and functional consequences. Physiol Rev 86: 369-408.
[3]. Hasan M T, Friedrich R W, Euler T, Larkum M E, Giese G, Both M, Duebel J, Waters J, Bujard H, Griesbeck O, Tsien R Y, Nagai T, Miyawaki A, Denk W. (2004) Functional fluorescent Ca²⁺ indicator proteins in transgenic mice under TET control. PLoS Biol 2: e163.
[4]. Nagai T, Yamada S, Tominaga T, Ichikawa M, Miyawaki A. (2004) Expanded dynamic range of fluorescent indicators for Ca(2+) by circularly permuted yellow fluorescent proteins. Proc Natl Acad Sci USA 101: 10554-10559.
[5]. Rudolf R, Mongillo M, Magalhaes P J, Pozzan T. (2004) In vivo monitoring of Ca(2+) uptake into mitochondria of mouse skeletal muscle during contraction. Cell Biol 166: 527-536.
[6]. Tallini Y N, Ohkura M, Choi B R, Ji G, Imoto K, Doran R, Lee J, Plan P, Wilson J, Xin H B, Sanbe A, Gulick J, Mathai J, Robbins J, Salama G, Nakai J, Kotlikoff M I. (2006) Imaging cellular signals in the heart in vivo: Cardiac expression of the high-signal Ca²⁺ indicator GCaMP2. Proc Natl Acad Sci U S A.
[7]. Miyawaki A, Llopis J, Heim R, McCaffery J M, Adams J A, Ikura M, Tsien R Y. (1997) Fluorescent indicators for Ca²⁺ based on green fluorescent proteins and calmodulin. Nature 388: 882-887.
[8]. Miyawaki A, Mizuno H, Nagai T, Sawano A. (2003) Development of genetically encoded fluorescent indicators for calcium. Methods Enzymol 360: 202-225.
[9]. Nagai T, Sawano A, Park E S, Miyawaki A. (2001) Circularly permuted green fluorescent proteins engineered to sense Ca2+. Proc Natl Acad Sci USA 98: 3197-3202.
[10]. Doyle T C, Burns S M, Contag C H. (2004) In vivo bioluminescence imaging for integrated studies of infection. Cell Microbiol 6: 303-317.
[11]. Bhaumik S, Gambhir SS. (2002) Optical imaging of Renilla luciferase reporter gene expression in living mice. Proc Natl Acad Sci USA 99: 377-382.
[12]. Iyer M, Salazar F B, Lewis X, Zhang L, Wu L, Carey M, Gambhir SS. (2005) Non-invasive imaging of a transgenic mouse model using a prostate-specific two-step transcriptional amplification strategy. Transgenic Res 14: 47-55.
[13]. Contag C H, Spilman S D, Contag P R, et al. (1997) Visualizing gene expression in living mammals using a bioluminescent reporter. Photochem Photobiol 66: 523-531.
[14]. Prasher D, McCann R O, Cormier M J. (1985) Cloning and expression of the cDNA coding for aequorin, a bioluminescent calcium-binding protein. Biochem Biophys Res Commun 126: 1259-1268.
[15]. Inouye S, Noguchi M, Sakaki Y, Takagi Y, Miyata T, Iwanaga S, Miyata T, Tsuji F I. (1985) Cloning and sequence analysis of cDNA for the luminescent protein aequorin. Proc Natl Acad Sci USA 82: 3154-3158.
[16]. Morise H, Shimomura O, Johnson F H, Winant J. (1974) Intermolecular energy transfer in the bioluminescent system of Aequorea. Biochemistry 13: 2656-2662.
[17]. Baubet V, Le Mouellic H, Campbell A K, Lucas-Meunier E, Fossier P, Brulet P. (2000) Chimeric green fluorescent protein-aequorin as bioluminescent Ca²⁺ reporters at the single-cell level. Proc Natl Acad Sci USA 97: 7260-7265.
[18]. Weissleder R, Ntziachristos V. (2003) Shedding light onto live molecular targets. Nat Med 9: 123-128.
[19]. Zhao H, Doyle T C, Coquoz O, Kalish F, Rice B W, Contag C H. (2005) Emission spectra of bioluminescent reporters and interaction with mammalian tissue determine the sensitivity of detection in vivo. J Biomed Opt 10: 41210.
[20]. Pfleger K D, Eidne K A. (2006) Illuminating insights into protein-protein interactions using bioluminescence resonance energy transfer (BRET). Nat Methods 3: 165-174.
[21]. Nagai T, Ibata K, Park E S, Kubota M, Mikoshiba K, Miyawaki A. (2002) A variant of yellow fluorescent protein with fast and efficient maturation for cell-biological applications. Nat Biotechnol 20: 87-90.
[22]. Campbell R E, Tour O, Palmer A E, Steinbach P A, Baird G S, Zacharias D A, Tsien R Y. (2002) A monomeric red fluorescent protein. Proc Natl Acad Sci U S A 99: 7877-7882.
[23]. Rogers K L, Stinnakre J, Agulhon C, Jublot D, Shorte S L, Kremer E J, Brulet P. (2005) Visualization of local Ca²⁺ dynamics with genetically encoded bioluminescent reporters. Eur J Neurosci 21: 597-610.
[24]. Bers D M, Patton C W, Nuccitelli R. (1994) A practical guide to the preparation of Ca²⁺ buffers. Methods Cell Biol 40: 3-29.
[25]. Allen D G, Blinks J R, Prendergast F G. (1977) Aequorin luminescence: relation of light emission to calcium concentration—a calcium-independent component. Science 195: 996-998.
[26]. Hastings J W, Mitchell G, Mattingly P H, Blinks J R, Van Leeuwen M. (1969) Response of aequorin bioluminescence to rapid changes in calcium concentration. Nature 222: 1047-1050.
[27]. Blinks J R, Wier W G, Hess P, Prendergast F G. (1982) Measurement of Ca²⁺ concentrations in living cells. Prog Biophys Mol Biol 40: 1-114.
[28]. Brini M, Marsault R, Bastianutto C, Alvarez J, Pozzan T, Rizzuto R. (1995) Transfected aequorin in the measurement of cytosolic Ca2+ concentration ([Ca2+]c). A critical evaluation. J Biol Chem 270: 9896-9903.
[29]. Badminton M N, Sala-Newby G B, Kendall J M, Campbell A K. (1995) Differences in stability of recombinant apoaequorin within subcellular compartments. Biochem Biophys Res Commun 217: 950-957.
[30]. Gorokhovatsky A Y, Marchenkov V V, Rudenko N V, Ivashina T V, Ksenzenko V N, Burkhardt N, Semisotnov G V, Vinokurov L M, Alakhov Y B. (2004) Fusion of Aequorea victoria GFP and aequorin provides their Ca2+-induced interaction that results in red shift of GFP absorption and efficient bioluminescence energy transfer. Biochem Biophys Res Commun 320: 703-711.
[31]. Nomura M, Inouye S, Ohmiya Y, Tsuji F I. (1991) A C-terminal proline is required for bioluminescence of the Ca(2+)-binding photoprotein, aequorin. FEBS Lett 295: 63-66.
[32]. Patterson G H, Piston D W, Barisas B G. (2000) Forster distances between green fluorescent protein pairs. Anal Biochem 284: 438-440.
[33]. Markova S V, Vysotski E S, Blinks J R, Burakova L P, Wang B C, Lee J. (2002) Obelin from the bioluminescent marine hydroid Obelia geniculata: cloning, expression, and comparison of some properties with those of other Ca2+-regulated photoproteins. Biochemistry 41: 2227-2236.
[34]. Rice B W, Cable M D, Nelson M B. (2001) In vivo imaging of light-emitting probes. J Biomed Opt 6: 432-440.
[35]. Shaner N C, Campbell R E, Steinbach P A, Giepmans B N, Palmer A E, Tsien R Y. (2004) Improved monomeric red, orange and yellow fluorescent proteins derived from Discosoma sp. red fluorescent protein. Nat Biotechnol 22: 1567-1572.
[36]. Shaner N C, Steinbach P A, Tsien R Y. (2005) A guide to choosing fluorescent proteins. Nat Methods 2: 905-909.
[37]. Inouye S, Shimomura O. (1997) The use of Renilla luciferase, Oplophorus luciferase, and apoaequorin as bioluminescent reporter protein in the presence of coelenterazine analogues as substrate. Biochem Biophys Res Commun 233: 349-353.
[38]. Zhao H, Doyle T C, Wong R J, Cao Y, Stevenson D K, Piwnica-Worms D, Contag C H. (2004) Characterization of coelenterazine analogs for measurements of Renilla luciferase activity in live cells and living animals. Mol Imaging 3: 43-54.
[39]. Galperin E, Verkhusha W, Sorkin A. (2004) Three-chromophore FRET microscopy to analyze multiprotein interactions in living cells. Nat Methods 1: 209-217.
U.S. Pat. No. 6,662,039 B2 Optical probing of neuronal connections with fluorescent indicators. 12/2003 Yuste et al.
Allen, D. G., Blinks, J. R., and Prendergast, F. G. (1977). Aequorin luminescence: relation of light emission to calcium concentration—a calcium-independent component. Science 195, 996-998.
Augustine, G. J., Santamaria, F., and Tanaka, K. (2003). Local calcium signaling in neurons. Neuron 40, 331-346.
Baron, K. T., Wang, G. J., Padua, R. A., Campbell, C., and Thayer, S. A. (2003). NMDA-evoked consumption and recovery of mitochondrially targeted aequorin suggests increased Ca²⁺ uptake by a subset of mitochondria in hippocampal neurons. Brain Res 993, 124-132.
Baubet, V., Le Mouellic, H., Campbell, A. K., Lucas-Meunier, E., Fossier, P., and Brulet, P. (2000). Chimeric green fluorescent protein-aequorin as bioluminescent Ca²⁺ reporters at the single-cell level. Proc Natl Acad Sci USA 97, 7260-7265.
Bauer, P. J. (2001). The local Ca concentration profile in the vicinity of a Ca channel. Cell Biochem Biophys 35, 49-61.
Brini, M., Marsault, R., Bastianutto, C., Alvarez, J., Pozzan, T., and Rizzuto, R. (1995). Transfected aequorin in the measurement of cytosolic Ca²⁺ concentration ([Ca²⁺]c). A critical evaluation. J Biol Chem 270, 9896-9903.
Brini, M., Pinton, P., Pozzan, T., and Rizzuto, R. (1999). Targeted recombinant aequorins: tools for monitoring [Ca²⁺] in the various compartments of a living cell. Microsc Res Tech 46, 380-389.
Christopherson, K. S., Sweeney, N. T., Craven, S. E., Kang, R., El-Husseini Ael, D., and Bredt, D. S. (2003). Lipid- and protein-mediated multimerization of PSD-95: implications for receptor clustering and assembly of synaptic protein networks. J Cell Sci 116, 3213-3219.
Conroy, W. G., Liu, Z., Nai, Q., Coggan, J. S., and Berg, D. K. (2003). PDZ-containing proteins provide a functional postsynaptic scaffold for nicotinic receptors in neurons. Neuron 38, 759-771.
DiGregorio, D. A., Peskoff, A., and Vergara, J. L. (1999). Measurement of action potential-induced presynaptic calcium domains at a cultured neuromuscular junction. J Neurosci 19, 7846-7859.
Emmanouilidou, E., Teschemacher, A. G., Pouli, A. E., Nicholls, L. I., Seward, E. P., and Rutter, G. A. (1999). Imaging Ca²⁺ concentration changes at the secretory vesicle surface with a recombinant targeted chameleon. Curr Biol 9, 915-918.
Etter E F, Minta A, Poenie M, Fay F S. (1996) Near-membrane [Ca²⁺] transients resolved using the Ca²⁺ indicator FFP18. Proc Natl Acad Sci USA. 93(11):5368-5373.
Eusebi, F., Miledi, R., Parker, I., Stinnakre, J. (1985) Post-synaptic calcium influx at the giant synapse of the squid during activation by glutamate. J. Physiol. 369, 183-197.
Fernandez-Chacon, R., Shin, O. H., Konigstorfer, A., Matos, M. F., Meyer, A. C., Garcia, J., Gerber, S. H., Rizo, J., Sudhof, T. C., and Rosenmund, C. (2002). Structure/function analysis of Ca²⁺ binding to the C2A domain of synaptotagmin 1. J Neurosci 22, 8438-8446.
Filippin, L., Magalhaes, P. J., Di Benedetto, G., Colella, M., and Pozzan, T. (2003). Stable interactions between mitochondria and endoplasmic reticulum allow rapid accumulation of calcium in a subpopulation of mitochondria. J Biol Chem 278, 39224-39234.
Goldberg, J. H., Tamas, G., Aronov, D., and Yuste, R. (2003). Calcium microdomains in aspiny dendrites. Neuron 40, 807-821.
Gorokhovatsky A Y., Marchenkov V V., Rudenko N V., Ivashina T V., Ksenzenko V N., Burkhardt N., Semisotnov G V., Vinokurov L M., Alakhov Y B. (2004) Fusion of Aequorea victoria GFP and aequorin provides their Ca(2+)-induced interaction that results in red shift of GFP absorption and efficient bioluminescence energy transfer. Biochem. Biophys. Res. Commun. 320(3):703-711. Hara M, Bindokas V, Lopez J P, Kaihara
Hara M, Bindokas V, Lopez J P, Kaihara K, Landa L R Jr, Harbeck M, Roe M W. (2004). Imaging endoplasmic reticulum calcium with a fluorescent biosensor in transgenic mice. Am J Physiol Cell Physiol. 287(4):C932-8.
Hasan M T, Friedrich R W, Euler T, Larkum M E, Giese G, Both M, Duebel J, Waters J, Bujard H, Griesbeck O, Tsien R Y, Nagai T, Miyawaki A, Denk W. 2004. Functional fluorescent Ca²⁺ indicator proteins in transgenic mice under TET control. PLOS Biol. 2(6):763-75.
Jaffe, L F (1993) Classes and mechanisms of calcium waves. Cell Calcium 14(10), 736-745.
Kandler K, Katz L C. (1998) Coordination of neuronal activity in developing visual cortex by gap junction-mediated biochemical communication. J. Neurosci. 18(4):1419-27.
Kendall J M, Sala-Newby G, Ghalaut V, Dormer R L, Campbell A K. (1992) Engineering the CA(2+)-activated photoprotein aequorin with reduced affinity for calcium. Biochem Biophys Res Commun. 187(2):1091-1097.
Knopfel T, Tomita K, Shimazaki R, Sakai R. (2003) Optical recordings of membrane potential using genetically targeted voltage-sensitive fluorescent proteins. Methods. 30(1): 42-8.
Kovalchuk, Y., Eilers, J., Lisman, J., and Konnerth, A. (2000). NMDA receptor-mediated subthreshold Ca(2+) signals in spines of hippocampal neurons. J Neurosci 20, 1791-1799.
Lidow, M. S. (2003). Calcium signaling dysfunction in schizophrenia: a unifying approach. Brain Res Brain Res Rev 43, 70-84.
Lipp P, Egger M, Niggli E. (2002). Spatial characteristics of sarcoplasmic reticulum Ca²⁺ release events triggered by L-type Ca²⁺ current and Na+ current in guinea-pig cardiac myocytes. J. Physiol. 15; 542(Pt 2):383-93.
Llinas, R., Sugimori, M., and Silver, R. B. (1995). The concept of calcium concentration microdomains in synaptic transmission. Neuropharmacology 34, 1443-1451.
Llinas, R., Sugimori, M., and Silver, R. B. (1992). Microdomains of high calcium concentration in a presynaptic terminal. Science 256, 677-679.
Marsault, R., Murgia, M., Pozzan, T., and Rizzuto, R. (1997). Domains of high Ca²⁺ beneath the plasma membrane of living A7r5 cells. Embo J 16, 1575-1581.
Mattson, M. P., and Chan, S. L. (2003). Neuronal and glial calcium signaling in Alzheimer's disease. Cell Calcium 34, 385-397.
Miller, A. L., Karplus, E., and Jaffe, L. F. (1994). Imaging [Ca²⁺]i with aequorin using a photon imaging detector. Methods Cell Biol 40, 305-338.
Miyawaki, A., Llopis, J., Heim, R., McCaffery, J. M., Adams, J. A., Ikura, M., and
Tsien, R. Y. (1997). Fluorescent indicators for Ca²⁺ based on green fluorescent proteins and calmodulin. Nature 388, 882-887.
Montero, M., Alonso, M. T., Carnicero, E., Cuchillo-Ibanez, I., Albillos, A., Garcia, A. G., Garcia-Sancho, J., and Alvarez, J. (2000). Chromaffin-cell stimulation triggers fast millimolar mitochondrial Ca²⁺ transients that modulate secretion. Nat Cell Biol 2, 57-61.
Montero, M., Alvarez, J., Scheenen, W. J., Rizzuto, R., Meldolesi, J., and Pozzan, T. (1997). Ca²⁺ homeostasis in the endoplasmic reticulum: coexistence of high and low [Ca²⁺] subcompartments in intact HeLa cells. J Cell Biol 139, 601-611.
Montero, M., Brini, M., Marsault, R., Alvarez, J., Sitia, R., Pozzan, T., and Rizzuto, R. (1995). Monitoring dynamic changes in free Ca²⁺ concentration in the endoplasmic reticulum of intact cells. Embo J 14, 5467-5475.
Morise, H., Shimomura, O., Johnson, F. H., Winant, J. (1974) Intermolecular Energy Transfer in the Bioluminescent system. Biochemistry, 13(12) 2656-2662.
Mothet J P, Fossier P, Meunier F M, Stinnakre J, Tauc L, Baux G. (1998) Cyclic ADP-ribose and calcium-induced calcium release regulate neurotransmitter release at a cholinergic synapse of Aplysia. J. Physiol. 507 (Pt 2):405-14.
Nagai T, Yamada S, Tominaga T, Ichikawa M, Miyawaki A. (2004) Expanded dynamic range of fluorescent indicators for Ca(2+) by circularly permuted yellow fluorescent proteins. Proc Natl Acad Sci USA. 101(29):10554-9.
Neher, E. (1998). Vesicle pools and Ca²⁺ microdomains: new tools for understanding their roles in neurotransmitter release. Neuron 20, 389-399.
Niedernberg A, Tunaru S, Blaukat A, Ardati A, Kostenis E. (2003) Sphingosine 1-phosphate and dioleoylphosphatidic acid are low affinity agonists for the orphan receptor GPR63. Cell Signal. 15(4): 435-46.
Nimchinsky, E. A., Yasuda, R., Oertner, T. G., and Svoboda, K. (2004). The number of glutamate receptors opened by synaptic stimulation in single hippocampal spines. J Neurosci 24, 2054-2064.
Peterlin Z A, Kozioski J, Mao B Q, Tsiola A, Yuste R. (2000) Optical probing of neuronal circuits with calcium indicators. Proc Natl Acad Sci USA. 97(7): 3619-24. Pivovarova, N. B., Pozzo-Miller, L. D., Hongpaisan, J., and Andrews, S. B. (2002). Correlated calcium uptake and release by mitochondria and endoplasmic reticulum of CA3 hippocampal dendrites after afferent synaptic stimulation. J Neurosci 22, 10653-10661.
Rizzuto, R., Simpson, A. W., Brini, M., and Pozzan, T. (1992). Rapid changes of mitochondrial Ca²⁺ revealed by specifically targeted recombinant aequorin. Nature 358, 325-327.
Shimomura O, Musicki B, Kishi Y, Inouye S. (1993) Light-emitting properties of recombinant semi-synthetic aequorins and recombinant fluorescein-conjugated aequorin for measuring cellular calcium. Cell Calcium. 14(5):373-8.
Shimomura O, Inouye S, Musicki B, Kishi Y. (1990) Recombinant aequorin and recombinant semi-synthetic aequorins. Cellular Ca²⁺ ion indicators. Biochem J. 270(2):309-12.
Shimomura O, Johnson F H. (1978) Peroxidized coelenterazine, the active group in the photoprotein aequorin. Proc Natl Acad Sci USA. 75(6): 2611-5.
Stosiek C, Garaschuk O, Holthoff K, Konnerth A. (2003) In vivo two-photon calcium imaging of neuronal networks. Proc Natl Acad Sci US. 100(12): 7319-24.
Stutzmann, G. E., Caccamo, A., LaFerla, F. M., and Parker, I. (2004). Dysregulated IP3 signaling in cortical neurons of knock-in mice expressing an Alzheimer's-linked mutation in presenilin1 results in exaggerated Ca²⁺ signals and altered membrane excitability. J Neurosci 24, 508-513.
Tang, T. S., Tu, H., Chan, E. Y., Maximov, A., Wang, Z., Wellington, C. L., Hayden, M. R., and Bezprozvanny, I. (2003). Huntingtin and huntingtin-associated protein 1 influence neuronal calcium signaling mediated by inositol-(1,4,5) triphosphate receptor type 1. Neuron 39, 227-239.
Varadi, A., and Rutter, G. A. (2002). Dynamic imaging of endoplasmic reticulum Ca²⁺ concentration in insulin-secreting MIN6 Cells using recombinant targeted chameleons: roles of sarco(endo)plasmic reticulum Ca²⁺-ATPase (SERCA)-2 and ryanodine receptors. Diabetes 51, S190-201.
Wang, G. J., Jackson, J. G., and Thayer, S. A. (2003). Altered distribution of mitochondria impairs calcium homeostasis in rat hippocampal neurons in culture. J Neurochem 87, 85-94.
Wu J Y, Lam Y W, Falk C X, Cohen L B, Fang J, Loew L, Prechtl J C, Kleinfeld D, Tsau Y. (1998) Voltage-sensitive dyes for monitoring multineuronal activity in the intact central nervous system. Histochem J. 30(3): 169-87.
Yu D, Baird G S, Tsien R Y, Davis R L. (2003) Detection of calcium transients in Drosophila mushroom body neurons with camgaroo reporters. J. Neurosci. 23(1):64-72.

1. A method for bioluminescence imaging in a biological system, wherein the method comprises:
(A) providing a biological system containing a transcriptionally active nucleic acid sequence encoding a Ca²⁺-sensitive polypeptide, or a Ca²⁺-sensitive polypeptide, which comprises a chemiluminescent protein linked to a fluorescent protein; and
(B) monitoring photons emitted by the Ca²⁺-sensitive polypeptide;
wherein the Ca²⁺-sensitive polypeptide comprises chemiluminescent protein sensitive to Ca²⁺ linked to a yellow fluorescent protein or a red fluorescent protein, and the link between the two proteins functions to allow luminescence by energy transfer between the two proteins.

2. The method according to claim 1, wherein the chemiluminescent protein, which is sensitive to Ca²⁺, is covalently linked to the yellow fluorescent protein or red fluorescent protein.

3. The method according to claim 1, wherein the chemiluminescent protein, which is sensitive to Ca²⁺, is aequorin.

4. The method according to claim 1, wherein the yellow fluorescent protein is the Venus yellow fluorescent protein.

5. The method according to claim 1, wherein the red fluorescent protein is mRFP1.

6. The method according to claim 1, wherein the photons emitted by the Ca²⁺-sensitive polypeptide are monitored in an animal or a plant.

7. The method according to claim 6, wherein the photons emitted by the Ca²⁺-sensitive polypeptide are monitored from deep tissues of an animal.

8. The method according to claim 7, wherein the tissue is a subthoracic tissue or a subcranial tissue.

9. The method of claim 6, wherein the animal is a mouse.

10. The method of claim 6, wherein the animal or plant is a transgenic animal or plant.

11. The method of claim 9, wherein the transgenic animal is a mouse.

12. A method for the optical detection of Ca²⁺ signals in a biological system, wherein the method comprises:
(A) providing a biological system containing a transcriptionally active nucleic acid sequence encoding a Ca²⁺-sensitive polypeptide, or a Ca²⁺-sensitive polypeptide, which comprises a chemiluminescent protein linked to a fluorescent protein; and
(B) monitoring photons emitted by the Ca²⁺-sensitive polypeptide;
wherein the Ca²⁺-sensitive polypeptide comprises a chemiluminescent protein which is sensitive to Ca²⁺, linked to a yellow fluorescent protein or red fluorescent protein, and the link between the two proteins functions to allow luminescence by energy transfer between the two proteins.

13. A method for the optical detection of Ca²⁺ signals in an animal, wherein the method comprises:
(A) providing a whole, live, animal containing a transcriptionally active nucleic acid sequence encoding a Ca²⁺-sensitive polypeptide, or a Ca²⁺-sensitive polypeptide, which comprises a chemiluminescent protein linked to a fluorescent protein; and
(B) non-invasively monitoring photons emitted by the Ca²⁺-sensitive polypeptide;
wherein the Ca²⁺-sensitive polypeptide comprises a chemiluminescent protein linked to a yellow fluorescent protein or a red fluorescent protein, and the link between the two proteins functions to allow transfer of energy by radiative or non-radiative intramolecular energy transfer.

14. The method according to claim 12 or 13, wherein the chemiluminescent protein, which is sensitive to Ca²⁺, is covalently linked to the yellow fluorescent protein or red fluorescent protein.

15. A method as claimed in claim 12 or 13, wherein the link between the two proteins functions to allow transfer of energy by Chemiluminescence Resonance Energy Transfer (CRET) between the two proteins.

16. The method according to claim 12 or 13, wherein the chemiluminescent protein, which is sensitive to Ca²⁺, is aequorin.

17. The method according to claim 12 or 13, wherein the yellow fluorescent protein is the Venus yellow fluorescent protein.

18. The method according to claim 12 or 13, wherein the red fluorescent protein is mRFP1.

19. The method according to claim 12 or 13, wherein the photons emitted by the Ca²⁺-sensitive polypeptide are monitored from deep tissues of an animal.

20. The method according to claim 19, wherein the tissue is a subthoracic tissue or a subcranial tissue.

21. The method according to claim 13, wherein the animal is a mouse.

22. The method according to claim 13, wherein the animal is a transgenic animal.

23. A method for the optical detection of Ca²⁺ signals in a transgenic mouse, wherein the method comprises:
(A) providing a freely moving, whole, live, transgenic mouse containing a transcriptionally active transgene encoding a Ca²⁺-sensitive polypeptide, which comprises a chemiluminescent protein linked to a fluorescent protein; and
(B) non-invasively monitoring photons emitted by the Ca²⁺-sensitive polypeptide;
wherein the Ca²⁺-sensitive polypeptide comprises aequorin protein covalently linked to a YFP (yellow fluorescent protein) or RFP (red fluorescent protein), and the link between the two proteins functions to allow transfer of energy by Chemiluminescence Resonance Energy Transfer (CRET) between the two proteins;
and wherein the photons are monitored from subthoracic tissue or subcranial tissue of the transgenic mouse.

24. The method according to claim 23, wherein the photons are monitored from deep tissues of the transgenic mouse.

25. The method according to claim 24, wherein the photons are monitored from subthoracic tissue or subcranial tissue of the transgenic mouse.

26. A method according to claim 23, wherein the photons are monitored during motion of the transgenic mouse.

27. A. method according to claim 23, wherein the aequorin has the substitution Asp407→Ala.

28. A method according to claim 23, which comprises, prior to the monitoring of photon emission, administering coelenterazine to the transgenic mouse to activate aequorin.

29. A method according to claim 23, wherein the aequorin is covalently linked to YFP and the photons are monitored from subthoracic tissue.

30. A method according to claim 23, wherein the subthoracic tissue comprises the heart.

31. A method according to claim 23, wherein the aequorin is covalently linked to RFP and the photons are monitored from subcranial tissue or liver.

32. A method according to claim 31, wherein the transgenic mouse is an adult transgenic mouse and the photons are monitored through the skull of the transgenic mouse.

33. A method according to claim 31, which comprises monitoring photons having a wavelength greater than about 600 nm emitted by the Ca²⁺-sensitive polypeptide.
